SEQUENCE LISTING (66 sequences)

SEQ ID NO: 1 - protein, 220 aa, synthetic construct
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 17 aa, synthetic construct
KSSQSVLYSSINKNYLA

SEQ ID NO: 3 - protein, 7 aa, synthetic construct
WASTRES

SEQ ID NO: 4 - protein, 9 aa, synthetic construct
QQYYSTPYT

SEQ ID NO: 5 - protein, 447 aa, synthetic construct
QLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGK

SEQ ID NO: 6 - protein, 10 aa, synthetic construct
GYGFITYWIG

SEQ ID NO: 7 - protein, 17 aa, synthetic construct
IIYPGDSETRYSPSFQG

SEQ ID NO: 8 - protein, 10 aa, synthetic construct
GSGISTPMDV

SEQ ID NO: 9 - protein, 465 aa, synthetic construct
QLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGKEGGGSGRERKARGGCPGS

SEQ ID NO: 10 - protein, 465 aa, synthetic construct
QLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGKEGGSSGRSRRARSVASQS

SEQ ID NO: 11 - protein, 214 aa, synthetic construct
DIQLTQSPSSLSASVGDRVTITCRASQTASSYLNWYQQKPGKAPNLLIYAASSLQSGVPSRFSGSGSVTDFTLTISSLQPEDFATYYCQQSYSTPPTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 11 aa, synthetic construct
RASQTASSYLN

SEQ ID NO: 13 - protein, 7 aa, synthetic construct
AASSLQS

SEQ ID NO: 14 - protein, 9 aa, synthetic construct
QQSYSTPPT

SEQ ID NO: 15 - protein, 452 aa, synthetic construct
QVQLVQSGSELKKPGASVKVSCKASGYTFATYALNWVRQAPGQGLEWMGWVNTNTGSPTYAQGFTGRFVFSFDTSVSTAYLQIRTLKAEDTAVYYCAVYYYDSGSPGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGK

SEQ ID NO: 16 - protein, 10 aa, synthetic construct
GYTFATYALN

SEQ ID NO: 17 - protein, 17 aa, synthetic construct
WVNTNTGSPTYAQGFTG

SEQ ID NO: 18 - protein, 13 aa, synthetic construct
YYYDSGSPGWFDP

SEQ ID NO: 19 - protein, 470 aa, synthetic construct
QVQLVQSGSELKKPGASVKVSCKASGYTFATYALNWVRQAPGQGLEWMGWVNTNTGSPTYAQGFTGRFVFSFDTSVSTAYLQIRTLKAEDTAVYYCAVYYYDSGSPGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGKEGGGSGRERKARGGCPGS

SEQ ID NO: 20 - protein, 214 aa, synthetic construct
DIQMTQSPSSLSASVGDRVTITCRASQNISSYLNWYQQEAGKAPKLLIYAASSLQSGVPSRFSGSGSGTDFTLTISSLQPEDFATYYCQQSYSTPRTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 11 aa, synthetic construct
RASQNISSYLN

SEQ ID NO: 22 - protein, 7 aa, synthetic construct
SYSTPRT

SEQ ID NO: 23 - protein, 455 aa, synthetic construct
QVQLVQSGAEVKKPGSSVKVSCKASGGTFSIYAITWVRQAPGQGLEWMGGIIPIIGTANYAQKFQGRVTITADKSTSTAYMELSSLRSEDTAVYYCARDFRYCSSTRCYFWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGK

SEQ ID NO: 24 - protein, 10 aa, synthetic construct
GGTFSIYAIT

SEQ ID NO: 25 - protein, 17 aa, synthetic construct
GIIPIIGTANYAQKFQG

SEQ ID NO: 26 - protein, 16 aa, synthetic construct
DFRYCSSTRCYFWFDP

SEQ ID NO: 27 - protein, 473 aa, synthetic construct
QVQLVQSGAEVKKPGSSVKVSCKASGGTFSIYAITWVRQAPGQGLEWMGGIIPIIGTANYAQKFQGRVTITADKSTSTAYMELSSLRSEDTAVYYCARDFRYCSSTRCYFWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGKEGGGSGRERKARGGCPGS

SEQ ID NO: 28 - protein, 216 aa, synthetic construct
QSALTQPPSASGTPGQRVTISCSGSSSNIGSNTVNWYQQLPGTAPKVLVYSNDQRPSGVPDRFSGSKSGTSASLAISGLQSEDEADYYCAAWDDSLNGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 29 - protein, 13 aa, synthetic construct
SGSSSNIGSNTVN

SEQ ID NO: 30 - protein, 7 aa, synthetic construct
SNDQRPS

SEQ ID NO: 31 - protein, 11 aa, synthetic construct
AAWDDSLNGPV

SEQ ID NO: 32 - protein, 455 aa, synthetic construct
QVQLVESGGGLVQPGGSLRLSCAASGFTFSSYAMSWVRQAPGKGLEWVSAISGSGDSTYYADSVKGRFTISRDNSKNTLYLQMNSLRAEDTAVYYCAKDRYYEFWSGYSNWFDPWGQGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGK

SEQ ID NO: 33 - protein, 10 aa, synthetic construct
GFTFSSYAMS

SEQ ID NO: 34 - protein, 17 aa, synthetic construct
AISGSGDSTYYADSVKG

SEQ ID NO: 35 - protein, 16 aa, synthetic construct
DRYYEFWSGYSNWFDP

SEQ ID NO: 36 - protein, 473 aa, synthetic construct
QVQLVESGGGLVQPGGSLRLSCAASGFTFSSYAMSWVRQAPGKGLEWVSAISGSGDSTYYADSVKGRFTISRDNSKNTLYLQMNSLRAEDTAVYYCAKDRYYEFWSGYSNWFDPWGQGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGKEGGGSGRERKARGGCPGS

SEQ ID NO: 37 - protein, 363 aa, synthetic construct
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 38 - protein, 18 aa, synthetic construct
RGRGSKRSEGRGRGCPGS

SEQ ID NO: 39 - protein, 8 aa, synthetic construct
PRRARSVA

SEQ ID NO: 40 - protein, 4 aa, synthetic construct
RKCR

SEQ ID NO: 41 - protein, 4 aa, synthetic construct
RRAR

SEQ ID NO: 42 - protein, 8 aa, synthetic construct
PGSAASVA

SEQ ID NO: 43 - protein, 4 aa, synthetic construct
GSAA

SEQ ID NO: 44 - protein, 18 aa, synthetic construct
EGGGSGRERKARGGCPGS

SEQ ID NO: 45 - protein, 18 aa, synthetic construct
EGGSSGRSRRARSVASQS

SEQ ID NO: 46 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 456 aa, synthetic construct
QVQLVQSGAEMKKPGASVKVSCKTSGYTFTNYKINWVRQAPGQGLEWMGWMNPDTDSTGYPQKFQGRVTMTRNTSISTAYMELSSLRSEDTAVYYCARSYGSGSYYRDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 236 aa, synthetic construct
MGWSCIILFLVATATGVHSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 465 aa, synthetic construct
MGWSCIILFLVATATGVHSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 50 - protein, 217 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 450 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLPGK

SEQ ID NO: 52 - protein, 9 aa, synthetic construct
SSGRSRRAR

SEQ ID NO: 53 - protein, 9 aa, synthetic construct
GRSRRARSV

SEQ ID NO: 54 - protein, 9 aa, synthetic construct
GRERKARGG

SEQ ID NO: 55 - protein, 18 aa, synthetic construct
EEDSPEMCRKCRTGCPRG

SEQ ID NO: 56 - protein, 18 aa, synthetic construct
EEDSPEMCRKRRTGCPRG

SEQ ID NO: 57 - protein, 18 aa, synthetic construct
EEDSPEMCRKARGGCPGS

SEQ ID NO: 58 - protein, 18 aa, synthetic construct
EEDSPEMCRRARSVASQS

SEQ ID NO: 59 - protein, 18 aa, synthetic construct
EGGGSGRERKARSVASQS

SEQ ID NO: 60 - protein, 18 aa, synthetic construct
EFFSSGRSRRARSVASQS

SEQ ID NO: 61 - protein, 18 aa, synthetic construct
EGSGSGRERKARGGCPGS

SEQ ID NO: 62 - protein, 6 aa, synthetic construct
EEDSPE

SEQ ID NO: 63 - protein, 6 aa, synthetic construct
MCRKCR

SEQ ID NO: 64 - protein, 6 aa, synthetic construct
TGCPRG

SEQ ID NO: 65 - protein, 725 aa, synthetic construct
DIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGSGGGSGGGSGGGGSGGGGGSGGGSGGGGSQVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNRFTQKSLSLSPGGEGGGSRSRKRRGGCSPG

SEQ ID NO: 66 - protein, 725 aa, synthetic construct
DIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGSGGGSGGGSGGGGSGGGGGSGGGSGGGGSQVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNRFTQKSLSLSPGGEGGGSRSRKRRGGCSPG

(19) United States
(12) Patent Application Publication
(10) Pub. No.: US 2025/0179151 A1
(43) Pub. Date: Jun. 5, 2025

(54) SPIKE FURIN CLEAVAGE IS A SARS-COV-2 TARGETING STRATEGY TO BREAK THE CHAIN OF INFECTION CYCLE

(71) Applicant: University of Virginia Patent Foundation, Charlottesville, VA (US)

(72) Inventor: Jogender Tushir-Singh, Davis, CA (US)

(73) Assignee: University of Virginia Patent Foundation, Charlottesville, VA (US)

(21) Appl. No.: 18/728,107

(22) PCT Filed: Jan. 12, 2023
(86) PCT No.: PCT/US2023/060595

§ 371 Date: Jul. 11, 2024

Related U.S. Application Data
(60) Provisional application No. 63/298,834, filed on Jan. 12, 2022.

(51) Int. Cl. C07K 16/10; A61K 39/00; A61P 31/14; C07K 16/18

(52) U.S. Cl. CPC C07K 16/1003; A61P 31/14; C07K 16/1063; C07K 16/18; A61K 2039/505; C07K 2317/51; C07K 2317/515; C07K 2317/56; C07K 2319/30

(57) ABSTRACT

Provided are methods for treating viral infections in subject in need thereof. In some embodiments, the method include administering to the subject a composition that has an effective amount of an agent that selectively interferes with host protease function to inhibit fusion-ready viral fragment generation, optionally S2 in case of SARS-CoV2 or GP160 or GP120 in case of HIV, and/or to destabilize a full-length viral fusion protein, optionally SARS-CoV-2 spike. Also provided are compositions that include an effective amount of an agent that selectively interferes with host protease function to inhibit fusion-ready viral fragment generation, optionally S2 in case of SARS-CoV2 or GP160 or GP120 in case of HIV, and/or to destabilize a full-length viral fusion protein, optionally SARS-CoV-2 spike, which compositions can optionally be employed in the disclosed methods.

### CROSS REFERENCE TO RELATED APPLICATION

The presently disclosed subject matter claims the benefit of U.S. Provisional Patent Application Ser. No. 63/298,834, filed Jan. 12, 2022, the disclosure of which incorporated herein by reference in its entirety.

### GOVERNMENT INTEREST

This invention was made with government support under Grant No. CA233752 awarded by the National Institutes of Health and under Grant Nos. W81XWH-18-1-0048 and W81XWH-19-1-0190 awarded by the Department of Defense. The government has certain rights in the invention.

### REFERENCE TO SEQUENCE LISTING XML

The Sequence Listing XML associated with the instant disclosure has been electronically submitted to the United States Patent and Trademark Office via the Patent Center as a 66,886 byte UTF-8-encoded XML file created on Jan. 12, 2023 and entitled “3062_177 PCT.xml”. The Sequence Listing submitted via Patent Center is hereby incorporated by reference in its entirety.

### TECHNICAL FIELD

The presently disclosed subject matter relates generally to compositions and methods for treating viral infections in subjects in need thereof. In some embodiments, the method comprise administering to a subject a composition comprising an effective amount of an agent that selectively interferes with host protease function to inhibit fusion-ready viral fragment generation.

### BACKGROUND

As of Dec. 1, 2021, SAR-CoV-2 has caused >262 million infections and >5.21 million deaths worldwide. Despite the remarkable speed of multiple vaccine approval by the USFDA, the detailed mechanism of SAR-CoV-2 cellular entry, chain of infectivity, and pathology remains unclear (Jackson et al., 2021). Thus, effective strategies targeting and interfering with the viral chain of infection before cellular entry remain pivotal for long-term therapeutic efficacy against continuously evolving SAR-CoV-2 mutant variants (Wang et al., 2021). Similar to SARS-CoV, SAR-CoV-2 entry into target lung cells is dependent on spike receptor-binding domain (RBD) interactions with ACE2 (Lan et al., 2020). However, unlike SARS-CoV, the SAR-CoV-2 spike protein harbors an arginine-rich multibasic site (S1/S2) between attachment (S1) and fusion (S2) domains (see FIGS. 1A and 1B). The cleavage of arginine-rich basic residues by host cellular furin protease is critical for efficient host cell membrane fusion during transmission chain (Cantuti-Castelvetri et al., 2020), SAR-CoV-2 cellular entry, and infection-induced cytopathic effects into human cells and tissues (Hoffmann et al., 2020a; Johnson et al., 2021). Experimentally tested host furin protease targeting chemical inhibitor drugs (Cheng et al., 2020), rationally designed de novo peptides, ACE2 traps (Glasgow et al., 2020), and mini proteins (Cao et al., 2020) has been shown effective in breaking chain of viral infection in cellular models. However, due to the lack of targeting specificity against spike-positive primary lung cells and infected tissues, if tested clinically, these protease inhibitory approaches are highly likely to interfere with the normal cellular processes in the body due to their random tissue distribution (Boesecke & Cooper, 2008).

The spike structural studies have demonstrated interactions of solvent-exposed furin cleaved region (S1/S2) with additional surface receptors to enhance viral cellular entry (Cantuti-Castelvetri et al., 2020; Wrapp et al., 2020) and disease pathogenicity (Hoffmann et al., 2020a; Johnson et al., 2021). Thus, selective targeting of furin in virus-producing cells remains one alternate strategy to break the SARS-CoV-2 infection cycle. One of the key barriers for lack of selectively targeted furin-inhibitor therapies (against SARS-CoV-2 infected and spike-producing cells) is the uncertainty of cellular organelle-specific furin mediated spike cleavage during viral cellular egress of virus (Cheng et al., 2020). In light of recent findings that β-coronaviruses such as SARS-CoV-2 uses lysosomal deacidification and potential late-recycling endosomal trafficking route for membrane egress rather than a trans-Golgi-network (TGN) secretory pathway (Ghosh et al., 2020), selective perturbation of furin function in spike escape route holds a key. Thus, targeting strategies capable of operating in cell membrane-endosome-lysosome networks are a rational start. As early, late, and recycling endosomes enriched immunoglobulin-G fragment crystallizable (Fc) receptor called neonatal receptor (FcRn) is vital in regulating the circulating level of antibodies (Oganesyan et al., 2014; Blumberg et al., 2019), IgG1-Fc mediated targeting of furin remains untested in the context of spike S1/S2 processing. The presently disclosed subject matter demonstrates biosynthetic secretory pathway independent S1/S2 cleavage of spike protein by furin. Importantly, to selectively target furin-driven spike processing of SARS-CoV-2, a simple, compelling, and targeted IgG1-Fc-based approach was engineered and described that interferes with the regulatory furin protease function to inhibit fusion-ready S2 fragment generation and to destabilize full-length spike (S0) protein.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter relates in some embodiments to methods for treating viral infections in subjects in need thereof. In some embodiments, the methods comprise, consist essentially of, or consist of administering to the subject a composition comprising an effective amount of an agent that selectively interferes with host protease function to inhibit fusion-ready viral fragment generation, optionally S2 in case of SARS-CoV2 or GP160 or GP120 in case of HIV, and/or to destabilize a full-length viral fusion protein, optionally SARS-CoV-2 spike. In some embodiments, the composition comprises an effective amount of an agent that selectively interferes with host furin protease function to inhibit fusion-ready S2 fragment generation and/or to destabilize full-length spike (S0) protein. In some embodiments, the viral infection is a HIV infection or a coronavirus infection, such as but not limited to Delta variant, Omicron variant, or Deltacron variant. In some embodiments, the viral infection is a SAR-CoV-2 infection, such as but not limited to Delta variant, Omicron variant, or Deltacron variant. In some embodiments, the agent comprises an Fc-conjugated furin competitive peptide and an antibody, optionally wherein the agent comprises a sequence as set forth in Table 3, or a biologically active fragment and/or homolog thereof, further optionally wherein the Fc-conjugated peptide comprises a sequence as set forth in Table 3 or a biologically active fragment and/or homolog thereof. In some embodiments, the agent comprises an Fc-conjugated furin competitive peptide and an antibody, and the Fc-conjugated furin competitive peptide is uncleavable by furin. In some embodiments, the agent comprises an Fc-conjugated furin competitive peptide and an antibody, wherein the Fc-conjugated furin competitive peptide is stable, flexible, and can be conjugated to any antibody targeting a virus. In some embodiments, the agent comprises an Fc-conjugated furin competitive peptide and an antibody, wherein the antibody Fc-conjugated furin competitive peptide is against furin recognition sequence of gp160 or gp120 of HIV or spike of SARS-CoV-2. In some embodiments, the antibody comprises a light chain variable region and a heavy chain variable region, and further wherein the light chain variable region comprises one of SEQ ID NOs: 1, 11, 20, and 28 or a sequence at least 95% identical thereto, and the heavy chain variable region comprises one of SEQ ID NOs: 5, 15, 23, and 32 or a sequence at least 95% identical thereto. In some embodiments, the antibody comprises a light chain variable region comprising SEQ ID NO: 1 and a heavy chain variable region comprising SEQ ID NO: 5; or the antibody comprises a light chain variable region comprising SEQ ID NO: 11 and a heavy chain variable region comprising SEQ ID NO: 15; or the antibody comprises a light chain variable region comprising SEQ ID NO: 20 and a heavy chain variable region comprising SEQ ID NO: 23; or the antibody comprises a light chain variable region comprising SEQ ID NO: 28 and a heavy chain variable region comprising SEQ ID NO: 32. In some embodiments, the heavy chain further comprises a C-terminal peptide selected from the group consisting of SEQ ID NOs: 44 and 45. In some embodiments, the antibody comprises a complement component antibody or a biologically active fragment and/or homolog thereof, optionally wherein the complement component antibody comprises an anti-C5 antibody or a biologically active fragment and/or homolog thereof, optionally wherein the complement component antibody comprises ravulizumab or eculizumab, or a biologically active fragment and/or homolog thereof, further optionally wherein the complement component antibody comprises SEQ ID NO: 65 and/or SEQ ID NO: 66, or a biologically active fragment and/or homolog thereof, further optionally wherein the biologically active fragment and/or homolog is at least 95% identical to SEQ ID NO: 65 or 66.

In some embodiments, the presently disclosed subject matter also relates to compositions comprising, consisting essentially of or consisting of an effective amount of an agent that selectively interferes with host protease function to inhibit fusion-ready viral fragment generation, optionally S2 in case of SARS-CoV2 or GP160 or GP120 in case of HIV, and/or to destabilize a full-length viral fusion protein, optionally SARS-CoV-2 spike. In some embodiments, the composition comprises, consists essentially of, or consists of an effective amount of an agent that selectively interferes with host furin protease function to inhibit fusion-ready S2 fragment generation and/or to destabilize full-length spike (S0) protein. In some embodiments, the agent comprises an Fc-conjugated furin competitive peptide and an antibody, optionally wherein the agent comprises a sequence as set forth in Table 3, or a biologically active fragment and/or homolog thereof, further optionally wherein the Fc-conjugated peptide comprises a sequence as set forth in Table 3, or a biologically active fragment and/or homolog thereof. In some embodiments, the agent comprises an Fc-conjugated furin competitive peptide and an antibody, and the Fc-conjugated furin competitive peptide is uncleavable by furin. In some embodiments, the agent comprises an Fc-conjugated furin competitive peptide and an antibody, and the Fc-conjugated furin competitive peptide is stable, flexible and can be conjugated to any antibody targeting a virus. In some embodiments, the agent comprises an Fc-conjugated furin competitive peptide and an antibody, and the antibody Fc-conjugated furin competitive peptide is against furin recognition sequence of gp160 or gp120 of HIV or spike of SARS-COV-2. In some embodiments, the antibody comprises a light chain variable region and a heavy chain variable region, and further wherein the light chain variable region comprises one of SEQ ID NOs: 1, 11, 20, and 28 or a sequence at least 95% identical thereto, and the heavy chain variable region comprises one of SEQ ID NOs: 5, 15, 23, and 32 or a sequence at least 95% identical thereto. In some embodiments, the antibody comprises a light chain variable region comprising SEQ ID NO: 1 and a heavy chain variable region comprising SEQ ID NO: 5; or the antibody comprises a light chain variable region comprising SEQ ID NO: 11 and a heavy chain variable region comprising SEQ ID NO: 15; or the antibody comprises a light chain variable region comprising SEQ ID NO: 20 and a heavy chain variable region comprising SEQ ID NO: 23; or the antibody comprises a light chain variable region comprising SEQ ID NO: 28 and a heavy chain variable region comprising SEQ ID NO: 32. In some embodiments, the heavy chain further comprises a C-terminal peptide selected from the group consisting of SEQ ID NOs: 44 and 45.

In some embodiments, the composition further comprises a pharmaceutically acceptable carrier, optionally a pharmaceutically acceptable carrier for use in a human.

In some embodiments, the presently disclosed compositions are for use in treating a viral infection. In some embodiments, the viral infection is a HIV infection or a coronavirus infection, such as but not limited to Delta variant, Omicron variant, or Deltacron variant. In some embodiments, the viral infection is a SAR-CoV-2 infection, such as but not limited to Delta variant, Omicron variant, or Deltacron variant. In some embodiments, the presently disclosed compositions are for use in treating COVID 19, including long COVID19

In some embodiments of the presently disclosed compositions, the antibody comprises a complement component antibody or a biologically active fragment and/or homolog thereof, optionally wherein the complement component antibody comprises an anti-C5 antibody or a biologically active fragment and/or homolog thereof, optionally wherein the complement component antibody comprises ravulizumab or eculizumab, or a biologically active fragment and/or homolog thereof, further optionally wherein the complement component antibody comprises SEQ ID NO: 65 and/or SEQ ID NO: 66, or a biologically active fragment and/or homolog thereof, further optionally wherein the biologically active fragment and/or homolog is at least 95% identical to SEQ ID NO: 65 or 66.

In some embodiments, the presently disclosed subject matter also relates to methods for treating COVID19 in a subject in need thereof, the method comprising administering to the subject a composition comprising an effective amount of an agent that selectively interferes with host protease function to inhibit fusion-ready viral fragment generation, optionally S2 in case of SARS-CoV2, and/or to destabilize a full-length viral fusion protein, optionally SARS-CoV-2 spike. In some embodiments, the composition comprises an effective amount of an agent that selectively interferes with host furin protease function to inhibit fusion-ready S2 fragment generation and/or to destabilize full-length spike (S0) protein. In some embodiments, the COVID19 is caused by a SARS-CoV-2 variant, such as but not limited to Delta variant, Omicron variant, or Deltacron variant. In some embodiments, the agent comprises an Fc-conjugated furin competitive peptide and an antibody, optionally wherein the agent comprises a sequence as set forth in Table 3, or a biologically active fragment and/or homolog thereof, further optionally wherein the Fc-conjugated peptide comprises a sequence as set forth in Table 3 or a biologically active fragment and/or homolog thereof. In some embodiments, the agent comprises an Fc-conjugated furin competitive peptide and an antibody, and the Fc-conjugated furin competitive peptide is uncleavable by furin. In some embodiments, the agent comprises an Fc-conjugated furin competitive peptide and an antibody, wherein the Fc-conjugated furin competitive peptide is stable, flexible, and can be conjugated to any antibody targeting a virus. In some embodiments, the agent comprises an Fc-conjugated furin competitive peptide and an antibody, wherein the antibody Fc-conjugated furin competitive peptide is against furin recognition sequence of spike of SARS-CoV-2. In some embodiments, the antibody comprises a light chain variable region and a heavy chain variable region, and further wherein the light chain variable region comprises one of SEQ ID NOs: 1, 11, 20, and 28 or a sequence at least 95% identical thereto, and the heavy chain variable region comprises one of SEQ ID NOs: 5, 15, 23, and 32 or a sequence at least 95% identical thereto. In some embodiments, the antibody comprises a light chain variable region comprising SEQ ID NO: 1 and a heavy chain variable region comprising SEQ ID NO: 5; or the antibody comprises a light chain variable region comprising SEQ ID NO: 11 and a heavy chain variable region comprising SEQ ID NO: 15; or the antibody comprises a light chain variable region comprising SEQ ID NO: 20 and a heavy chain variable region comprising SEQ ID NO: 23; or the antibody comprises a light chain variable region comprising SEQ ID NO: 28 and a heavy chain variable region comprising SEQ ID NO: 32. In some embodiments, the heavy chain further comprises a C-terminal peptide selected from the group consisting of SEQ ID NOs: 45 and 46. In some embodiments, the antibody comprises a complement component antibody or a biologically active fragment and/or homolog thereof, optionally wherein the complement component antibody comprises an anti C5 antibody or a biologically active fragment and/or homolog thereof, optionally wherein the complement component antibody comprises ravulizumab and eculizumab, or a biologically active fragment and/or homolog thereof, further optionally wherein the complement component antibody comprises SEQ ID NO: 65 and/or SEQ ID NO: 66, or a biologically active fragment and/or homolog thereof, such as a sequence at least 95% identical thereto. In some embodiments, the COVID19 is Long COVID19.

Accordingly, it is an object of the presently disclosed subject matter to provide methods and compositions for treating viral infections, such as HIV or coronavirus infections, such as SARS-CoV-2 infections. This and other objects are achieved in whole or in part by the presently disclosed subject matter.

Further, an object of the presently disclosed subject matter having been stated above, other objects and advantages of the presently disclosed subject matter will become apparent to those skilled in the art after a study of the following description, EXAMPLES, and Figures.

### BRIEF DESCRIPTIONS OF THE FIGURES

FIGS. 1A-1K. Design of furin competitive FuG1 strategy. (FIGS. 1A and 1B) Schematic of SARS-CoV-2 and SARS-CoV show RBD domain, S1/S2, and S2′ sites. (FIG. 1C) Ribbon structure of SARS-CoV-2 spike monomer (PDB: 6ZGI). Red spheres (when shown in color): S1/S2 residues near furin cleavage site. Yellow spheres (when shown in color): S2′ residues. RBD domain is in green (when shown in color). (FIG. 1D) 22-IgG1 (PDB: 6W41) half-body schematic inserted with Fc extendable linkers harboring competitive furin engaging residues (pink when shown in color, cartoon only). (FIG. 1E) Schematic showing competitively engaged active site cleft of furin (PDB: 6HZD) with Fc extended peptide (pink when shown in color) of 22-FuG1 antibody. 22-FuG's half body is shown. When shown in color, 22-FuG1 VH: Gold, 22-FuG1 VL: Gray, Furin: Blue, 22-FuG1 Fc-extended peptide: Pink. (FIG. 1F) Sequences of FuG1 (optimal competitive lead; SEQ ID NO: 44) and cFuGI (control cleavable; SEQ ID NO: 45) Fc-extended linkers. (FIG. 1G) Cleavage score of FuG1 and cFuG1 Fc-extended linkers (SEQ ID NOs: 44 and 45, respectively) based on PiTou algorithm. (FIG. 1H) The amino acid sequence of human IgG4-Fc tagged recombinant DR5 (SEQ ID NO: 37). Sequence in bold represents 101 amino acids (˜12 kDa) at the N-terminus of DR5 and is N-terminal to the RKCR tetrapeptide (SEQ ID NO: 40) of DR5 (in a box) and sequence in black is 261 amino acids (˜30 kDa), C-terminal to the RKCR tetrapeptide (SEQ ID NO: 40). Amino acids in box (RKCR tetrapeptide; SEQ ID NO: 40) were replaced with the corresponding sequences shown in FIG. 1I to generate various IgG4-Fc tagged DR5 proteins. Upon furin cleavage, N-terminal (bold, ˜12-15 kDa), C-terminal (Black: 261 amino acids, ˜27 kDa). See also FIGS. 11-1K. (FIG. 1I) Amino acids in under P>6 (blue when shown in color), under P1-P6 (red when shown in color), and under P1′—P6′ (black) were inserted to replace the corresponding sequence around RKCR residues (SEQ ID NO: 40) to generate various IgG4-Fc tagged DR5 proteins, labeled in left column. Residues in P1-P6 are part of the core furin substrate site, P>6 represent residues upstream of the core furin substrate site and P1′—P6′ represent residues downstream of the core furin substrate site. Furin cleavage score based on PiTou tool composed on hidden Markov model is shown for each 18 amino acid sequence. (FIG. 1J) Based on PiTou score 4 different IgG4-Fc tagged DR5 with indicated substitutions were expressed, purified, and dialyzed in PBS, followed by cleavage analysis using 10 ng recombinant furin (see FIG. 1H). Lane 1 and lane 2 (see FIG. 1I) showed expected furin cleavage with the release of IgG4-Fc tagged DR5 ˜30 kDa and ˜12 kDa fragments, while recombinant DR5 proteins in lane 3 and 4 with substituted sequences (see FIG. 11) were not cleaved by recombinant furin. (FIG. 1K) Same as FIG. 1J, except 50 ng furin was used along with 20 nM KCl (pH 6.7) in the cleavage reaction buffer. See FIGS. 1H and 1I.

FIGS. 2A-2C. Furin inhibitory function and flexibility of Fc-extended FuG1 peptide strategy. (FIG. 2A) The binding kinetics of immobilized biotinylated furin against indicated farletuzumab-FuG1, farletuzumab-cFuG1, and Farletuzumab-IgG1 were measured using BLI. (FIGS. 2B and 2C) FuG1 and cFuG1 antibodies were tested using a competitive inhibitory furin assay kit, which measures fluorescent signal as an indicator of the protease activity kinetics. PBS and assay kit supplied inhibitor (chloromethylketone) plots show optimal and competitively inhibited furin activity range with the supplied substrate. Farletuzumab-cFuG1 and farletuzumab FuG1 with indicated concentrations were added to competitively inhibit furin activity in presence of supplied substrate, followed by readout of plates for fluorescent signal using microplate reader at 380 nm/460 nm (see Materials and Methods Employed in the EXAMPLES section herein below).

FIGS. 3A-3H. TGN independent cleavage of spike. (FIG. 3A) 293-ACE2 cells were treated with indicated Brefeldin-A concentrations at the time of spike transfection. 24 hours later, lysates were analyzed for spike and DR5 using immunoblotting. (FIGS. 3B-3D) Cells grown in 150 cm² dishes (divided in groups as indicated on top) were transfected with the spike. 24 hours later, ER and Golgi fractions were enriched using Minute ER and Golgi enrichment kits (see methods) as indicated on top. Lysates were subjected to immunoblotting with indicated ER and Golgi resident markers along with spike. (FIGS. 3E and 3F) Same as FIGS. 3B-3D, except along with spike, GFP (with N-terminal membrane sorting peptide) was used as a positive control for traditional TGN sorting. (FIG. 3G) Schematic of crude endosome (CE) enrichment assay for data shown in FIG. 3H (see Materials and Methods Employed in the EXAMPLES section herein below). (FIG. 3H) Along with spike and spike+GFP total lysates, enriched CE from spike+GFP co-transfected cell were analyzed for indicated endosomal, lysosomal marker using immunoblotting.

FIGS. 4A-4O. Fc-extended peptide does not interfere with target binding and activity FuG1 antibodies. (FIG. 4A) Genetic construction schematic of 22-IgG1, 22-FuG1. Fc extended FuG1 peptide is shown in green when shown in color. (FIG. 4B) A reducing SDS-Gel of 22-IgG1 (1) and 22-FuG1 (2). Arrows depict the difference in the sizes of IgG1 and FuG1 VH. (FIG. 4C) A reducing SDS-Gel of 6.30 and 12.25 IgG1 and FuG1 antibodies. Open arrows show the difference in the sizes of IgG1 and FuG1 VH. (FIG. 4D) Cell viability assay of KMTR2-IgG1, KMTR2-FuG1, and KMTR2-FuG1 with linkered Fab against ovarian cancer cells. (FIG. 4E) Cell viability assay of trastuzumab-IgG1, trastuzumab-FuG1 and trastuzumab-FuG1 with linkered Fab against breast cancer cells. (FIG. 4F) IgG4-Fc tagged rFOLR1 antigen was coated on 96 well plates overnight. Coated plates were treated with the increasing concentrations of either farletuzumab-IgG1 or farletuzumab-FuG1 and IgG1 control antibodies as indicated. Following numerous washes, the HRP conjugated secondary antibody that is specific to IgG1-Fc (but not IgG4-Fc) was used to measure the binding strength using TMB substrate and ELISA plate reader capable of reading at 450 nm (n=3). (FIGS. 4G and 4H) The binding kinetics of immobilized biotinylated recombinant DR5-IgG4-Fc and RBD IgG4-Fc against indicated antibodies were measured using BLI. Table provided below both figures. (FIG. 4I) Schematic of the experiment shown in FIG. 4J. (FIG. 4J) DR5 expressing ovarian (OVCAR-3) tumor cells were treated with indicated antibodies for 30 minutes in cross-linking conditions. Immunoprecipitation (IP) was carried out using IgG1-Fc specific magnetic beads followed by immunoblotting against DR5, furin. (FIG. 4K) Surface expression analysis of ACE2 on 293-ACE2, 293-WT, VERO-E6 and Calu-3 cells using flow cytometry. (FIGS. 4L and 4M) Spike transfected and untransfected (+/−) 293-ACE2 cells were treated with indicated antibodies followed by IP as described in FIG. 4J. (FIG. 4N) A 22-FuG1-mutant in FIG. 4M contain random charge control (RCC; SEQ ID NO: 38) in Fc-peptide with the sequence shown at the bottom. (FIG. 4O) Same as FIG. 4M, except instead of 22-FuG1, 6.30 FuG1 was used and instead of 293-ACE2 cells, HEK-293 cells were used.

FIGS. 5A-5J. FuG1 strategy interferes with S1/S2 cleavage in spike transfected cells. (FIG. 5A) Left: Schematic of SARS-CoV-2 showing RBD (green when shown in color) and non-RBD binding antibodies (blue when shown in color). Right: Schematic of SARS-CoV-2 showing wild type S1/S2 sequence (PRRARSVA (SEQ ID NO: 39), with the tetrapeptide RRAR (SEQ ID NO: 41) in bold) and RRAR mutant sequence (PGSAASVA (SEQ ID NO: 42), with the mutated tetrapeptide GSAA (SEQ ID NO: 43) in bold; referred to herein as ARRAR). (FIG. 5B) 4 hours post-WT spike DNA transfection, indicated antibodies were added onto the HEK-293 cells (70-80% confluent) and lysates were prepared after 24 hours post-transfection. For control Furin mutant ΔRRAR spike plasmid was used. (FIG. 5C) Same as FIG. 5B, except ACE2-stable HEK-293 cells were used, and a random charge control antibody was included. (FIG. 5D) same as FIG. 5C except Calu3 cells were tested. (FIG. 5E) same as FIG. 5C except 6.30-IgG1 and 6.30-FuG1 antibodies were used. In lane 4, 5-fold KMTR2 was added along with 6.30-FuG1 and in lane 5, 5-fold 6.30-IgG1 was added to significantly out-compete with 6.30-FuG1 binding with spike. (FIGS. 5F and 5G) Same as FIG. 5E, except ACE2-stable HEK-293 cells were used, and instead of 22-FuG1, other spike RBD targeting (6.29-IgG1, 6.29-FuG1, 6.30-IgG1, 6.30-FuG1), and non-RBD targeting (12.19-FuG1, 12.25-FuG1) antibodies were used as indicated on the top. Murine DR5 targeting MD5-1 is specificity control. Tubulin is loading control and higher exposure blots are also shown. (FIG. 5H) Spike transfected 293-ACE2 cells were treated with indicated antibodies 6 hours post spike transfection. 24 hours later, the number of syncytia was counted after various indicated treatments. Error bar indicated SEM (n=3). (FIG. 5I) Growing VERO-E6 cells were transfected with 1.0 μg WT spike (expression vector. After 4 hours of transfection, IgG1 control, 22-IgG1, 22-FuG1 and 22-FuG1-Lin (100 μg) were added to media. After 24 hours, the number of syncytia were counted (using EVOS imaging system) and plotted. Data indicated SEM (n=3). (FIG. 5J) Same as FIG. 5I, except the percent surface expression of spike positive cells was analyzed after 48 hours using flow cytometry. Data in FIGS. 5H-5J represent 3 independent experiments. For statistical analysis, unpaired T-test with Welch's correction was employed (** p<0.005, *** p<0.0001) and each immunoblot is representative of 2 or 3 set of experiments.

FIGS. 6A-6L. In addition of S1/S2 cleavage inhibition, FuG1 antibodies destabilize S0 for particle incorporation in virus-producing cells. (FIG. 6A) Schematic of generation of WT and ΔRRAR spike expressing pseudovirus. (FIG. 6B) VSV-G, SARS-CoV and WT or furin mutant SARS-CoV-2 spike transfected BHK 1 cells were added with replication-restricted G*ΔG-luciferase-rVSV particles. 48 hours after addition of the particles, pseudovirions were harvested. Harvested pseudovirions were analyzed for S0 and S2 fragments using immunoblotting. (FIG. 6C) Harvested pseudoviral particles bearing WT spike proteins were incubated with indicated antibodies for 1 hour at 37, followed by transduction of 293-ACE2 cells. After 24 hours, a virus-encoded luciferase signal was measured from the lysates. ΔRRAR pseudoviral particles were used as control. Error bar indicated SEM (n=3). (FIG. 6D) Schematic workflow of experiments shown in FIG. 6E, FIG. 6F, and FIG. 6G. (FIG. 6E) Same as FIG. 6B, except similar to 293-ACE2 transfection experiments (see FIGS. 4B and 4C), spike transfected BHK1 cells were pre-treated with indicated antibodies during pseudoviral production. (FIG. 6F) WT Spike transfected BHK1 cells were treated with indicated antibodies (4 hours post transfection) during pseudoviral production. 48 hours after addition of the particles, pseudovirions were harvested. Harvested pseudovirions were analyzed for S0 and S2 fragments using immunoblotting. VSV-M is loading control. (FIG. 6G) WT Spike transfected VERO-E6 cells were treated with indicated antibodies for 48 hours, followed by immunoblotting. (FIG. 6H) WT and ΔRRAR Spike transfected 293-ACE2 cells were treated with indicated antibodies (4 hours post transfection). 24 hours after lysates were run under non-reducing denaturing conditions. Asterisk marked lane 3 showed slow-migrating large size aggregates. (FIG. 6I) Same as FIG. 6H, except along with 22-FuG1, 6.30-FuG1 and a control non-target (the anticoagulant drug PRADAXA®; ethyl N-[(2-{[(4-{N′-[(hexyloxy) carbonyl]carbamimidoyl}phenyl)amino]methyl}-1-methyl-1H-benzimid-azol-5-yl) carbonyl]-N-2-pyridinyl-β-alaninate; CAS Number 211915 Jun. 9) binding Idarucizumab-FuG1 was used. Asterisk marked lanes 2, 4, and 5 showed slow-migrating large size aggregates. (FIG. 6J) Same as FIGS. 6E and 6F, except multiple additional controls were used including Idarucizumab-FuG1 during spike bearing pseudoviral generation. (FIGS. 6K and 6L) Same as FIGS. 6E, 6F, and 6J, except in two independent biological experiments 4-fold and 6-fold concentrated lysates were loaded on the gel in case of pseudoviral generated in presence of FuG1 antibodies. VSV-M is loading control, and each immunoblot is representative of 2 or 3 set of experiments.

FIGS. 7A-7I. Spike S0 destabilization by FuG1 strategy is proteasome dependent. (FIG. 7A) Workflow of data shown in FIG. 7B. (FIG. 7B) Spike transfected, non-FuG1 treated (lanes 1, 2) and spike transfected plus 22-FuG1 treated (lanes 4, 6) were subjected to immunoblotting analysis of spike #MG-132 (10 μM). Lanes 3 and 5 were loaded with control ER-enriched lysates (5 μg) to confirm if equal amount of translating spike protein was present #MG-132 proteasome inhibitor in FuG1 treated samples. (FIG. 7C) Spike, S0 signal intensity in total lysates (±MG-132 treated) after normalization with GAPDH is shown. (FIG. 7D) 4 hours post-WT spike DNA transfection, indicated antibodies (on top) were added onto the 293-ACE2 cells±increasing (20 μM and 50 μM) MG-132 concentrations. Lysates were prepared 36 or 48 hours post-transfection following by immunoblotting against spike. (FIG. 7E) Experimental control to confirm that MG-132 treatment does not interfere with WT spike processing and non-processing of ΔRRAR spike. (FIG. 7F) WT spike transfected BHK1 cells were pre-treated with indicated antibodies (±20 μM and 50 μM MG-132 or PBS alone) after 4 hours. 12 hours later, replication-restricted G*ΔG-luciferase-rVSV particles were added. 48 hours later, harvested pseudovirions lysates were analyzed for S0 and S2 fragments using immunoblotting on viral particles. VSV-M and a nonspecific band in spike blot was loading control. (FIGS. 7G-71) Same as FIGS. 6E, 6F, 6J, 6K, and 6L, except spike bearing pseudoviral particles harvested in the presence of 22-IgG1 and 22-FuG1 antibodies were transduced onto 293-ACE2 cells (TMPRSS2⁻ cells in FIG. 7G and FIG. 7I) and Calu-3 cells (TMPRSS2⁺ cells in FIG. 7H). After 24 hours, virus-encoded luciferase signal was quantitated from the lysates as a measure of host cell entry. ΔRRAR pseudoviral particles were used as control for both TMPRSS2⁻ and TMPRSS2⁺ cells as described in Hoffmann et al., 2020a. Error bars indicates SEM (n=3). Statistical significance in FIGS. 7G-7I was defined by unpaired T-test with Welch's correction (*: p<0.05; **: p<0.005; ***: p<0.0001) and immunoblots are representative of 2 or 3 independent experiments.

FIGS. 8A-8H. Selectivity of FuG1 strategy against spike expressing cells. (FIG. 8A) Flow cytometry analysis of surface FOLR1 in HEK-293 cells. (FIG. 8B) Flow cytometry confirmation of similar level of FOLR1 expression in 293-ACE and HCC-1806 cells. (FIG. 8C) Schematic of the co-culture experiment described in FIG. 8E and FIG. 8F. (FIG. 8D) Schematic showing preferred binding of 22-FuG1 (hatched) toward high spike expressing 293-ACE2 cells, while farletuzumab-FuG1 (Farle-FuG1, solid) distributes equally against both cell lines expressing similar levels of FOLR1 (see FIG. 8B). (FIGS. 8E and 8F) 4 hours after spike-EGFP construct transfection, co-cultured cells were treated with 22-IgG1, farletuzumab-FuG1 (Farle-FuG1) and 22-FuG1 antibodies. 30 hours later cells were imaged at 10× magnification using Evos fluorescent microscope. (FIG. 8G) Number of syncytia from F. Error bar indicated SEM (n=3). (FIG. 8H) Same experiment as in FIGS. 8E-8G, except additional internal transfection control HA-tagged murine-DR5 (HA-muDR5-pCDNA3.1) was co-transfected with spike-EGFP construct. 48 hours later total lysates from spike-transfected 293-ACE2 cells alone, and cocultured-cells were subjected to lysate preparation, followed by immunoblotting analysis of S0 and S2. HA-muDR5 served an internally equivalent transfection efficiency control and tubulin is lysate loading control. Statistical significance in FIG. 8G was defined by unpaired T-test (*: p<0.05; **: p<0.005).

FIGS. 9A-9B. Working mechanism of FuG1 strategy. (FIG. 9A) In SARS-CoV-2 producing cells, after RNA packing and viral structural assembly in endoplasmic-reticulum Golgi-intermediate compartment (ERGIC), the spike harboring RNA capsid assembly complex is shuttled towards deacidified lysosomal, late, early, recycling or membrane endosomes route (see Ghosh et al., 2020) rather than TGN secretory pathway (see FIGS. 3A-3H). The endosomal furin mediates proteolytic processing of spike S1/S2 before viral release. If cell surface enriched furin also contributes to spike cleavage could not be ruled out. Nonetheless, furin cleavage generates fusion-ready S2 fragment which allows spike to effectively bind NRP1 (Cantuti-Castelvetri et al., 2020) to enhance overall viral cellular entry in ACE2⁺ acceptor cells. (FIG. 9B) Membrane and endosomal recycling and spike targeting FuG1 antibodies makes use of spike binding as an anchor to competitively inhibit furin proteolytic processing of spike S1/S2 in the membrane-endosomal network. Furthermore, large size complex aggregates (slower migrating on gel) are generated potentially due to tripartite SARS-CoV-2 spike-FuG1-furin multivalent interactions (see FIGS. 6A-6L). The resulting higher-order aggregates are potentially degraded by proteasome complex (see FIGS. 7A-7I) and additional unknown mechanisms. The latter collectively reduces and destabilizes overall S0 levels on released viral particles (see FIGS. 5A-5J and 6A-6L). Collectively, both suppression of S0 to S2 generation (B1) and S0 destabilization (B2) limit spike particle incorporation and subsequent viral entry and infection in ACE2⁺ acceptor cells.

FIGS. 10A to 10E. Engineering of CI_FuG1 antibody. (FIGS. 10A and 10B) Schematic and domain organization of a spike RBD (blue when shown in color) and C5 (red when shown in color) targeting antibodies. (FIG. 10C) Schematic of bispecific antibody with inability to incorporate Fc extended FuG1 peptide. (FIG. 10D) Schematic of a knob into hole mutations (see Shivange et al., 2018) harboring dual specificity antibody to allow heterodimerization of two IgG chains (spike RBD, blue when shown in color and C5, red when shown in color) that only differ in Fv domain. The dotted line represented by glycine-serine linkers genetically links the 3′ end of c-kappa and 5′ end of VH for proper light chain pairing (Shivange et al., 2018). In addition, C1_FuG1 antibody contains genetically linked Fc-extended furin competitive peptides as described elsewhere herein. (FIG. 10E) Reducing gel of CI_FuG1 with control IgG1 (see FIG. 10A) and non-FuG1 peptide harboring bispecific antibody (see FIG. 10C). As seen on reducing gel, only CI_FuG1 did not contain VL chain.

FIGS. 11A and 11B. FuG1 is significantly effective over RBD neutralizing spike IgG1 (FIG. 11A) Vero E6 cells were infected with 153 PFU of mNeonGreen SARS-CoV-2 (Wuhan stain) for 6 hours, followed by treatment with FuG1 or IgG antibodies. Untreated cells showed robust levels of infection, as green fluorescence of infection was readily detected under the microscope on day 2 post-infection. In comparison, both RBD neutralizing IgG and FuG1 at 100 ng/mL dramatically inhibited the SARS-CoV-2 infection on day 4, indicated by a pronounced reduction in SARS-CoV-2 infection signals of mNeonGreen. Moreover, almost no spike and viral infection signals were observed in Vero E6 cells treated with 1 and 10 μg/mL FuG1. (FIG. 11B) Vero E6 cells and culture supernatant were fixed and processed for subsequent assays in the BSL2 laboratory. Real-time PCR assays evaluated SARS-CoV-2 viral spike RNA output. Treatment with 100 ng/mL FuG1 and IgG resulted in 3-4 fold reduction in viral RNA levels compared to untreated samples. Moreover, treatment with 10 μg/mL FuG1 and IgG demonstrated significant 6-log reductions in RNA copies of Vero E6 cells, which were close to the levels of detection limit per our real-time PCR assays. Importantly unlike IgG1, FuG1 showed significant 6-log reductions even at 1 μg/mL

FIG. 12. In vivo study design. Randomly selected 6-12 weeks old B6J.Cg. Tg K18-hACE2 mice (equal number of male and female) are inoculated with 104 PFU virus and are treated with indicated antibodies. From DO to D6, mice weights are monitored (euthanasia criteria: 80% starting weight, ataxia, or moribund). Animals are euthanized on day 6 followed by tissue collection for virological and histology analysis. D3-D6 indicates days from 3 days before and 6 days after viral inoculation.

### BRIEF DESCRIPTIONS OF THE SEQUENCE LISTING

SEQ ID NO: 1 is the amino acid sequence of the variable light chain region of exemplary antibody 22 of the presently disclosed subject matter.

SEQ ID NOs: 2-4 are the amino acid sequences of the complementarity determining regions (CDRs) 1-3, respectively, of the variable light chain region of exemplary antibody 22 of the presently disclosed subject matter.

SEQ ID NO: 5 is the amino acid sequence of the variable heavy chain region of exemplary antibody 22 of the presently disclosed subject matter.

SEQ ID NOs: 6-8 are the amino acid sequences of the complementarity determining regions (CDRs) 1-3, respectively, of the variable heavy chain region of exemplary antibody 22 of the presently disclosed subject matter.

SEQ ID NO: 9 is the amino acid sequence of the variable heavy chain region of exemplary antibody 22 of the presently disclosed subject matter with a C-terminal FuG1 peptide (SEQ ID NO: 45).

SEQ ID NO: 10 is the amino acid sequence of the variable heavy chain region of exemplary antibody 22 of the presently disclosed subject matter with a C-terminal cFuG1 peptide (SEQ ID NO: 46).

SEQ ID NO: 11 is the amino acid sequence of the variable light chain region of exemplary antibody 6.29 of the presently disclosed subject matter.

SEQ ID NOs: 12-14 are the amino acid sequences of the complementarity determining regions (CDRs) 1-3, respectively, of the variable light chain region of exemplary antibody 6.29 of the presently disclosed subject matter.

SEQ ID NO: 15 is the amino acid sequence of the variable heavy chain region of exemplary antibody 6.29 of the presently disclosed subject matter.

SEQ ID NOs: 16-18 are the amino acid sequences of the complementarity determining regions (CDRs) 1-3, respectively, of the variable heavy chain region of exemplary antibody 6.29 of the presently disclosed subject matter.

SEQ ID NO: 19 is the amino acid sequence of the variable heavy chain region of exemplary antibody 6.29 of the presently disclosed subject matter with a C-terminal FuG1 peptide (SEQ ID NO: 44).

SEQ ID NO: 20 is the amino acid sequence of the variable light chain region of exemplary antibody 6.30 of the presently disclosed subject matter.

SEQ ID NOs: 21 and 22 are the amino acid sequences of the complementarity determining regions (CDRs) 1 and 3, respectively, of the variable light chain region of exemplary antibody 6.30 of the presently disclosed subject matter. CDR2 of the light chain region of exemplary antibody 6.30 of the presently disclosed subject matter is SEQ ID NO: 13.

SEQ ID NO: 23 is the amino acid sequence of the variable heavy chain region of exemplary antibody 6.30 of the presently disclosed subject matter.

SEQ ID NOs: 24-26 are the amino acid sequences of the complementarity determining regions (CDRs) 1-3, respectively, of the variable heavy chain region of exemplary antibody 6.30 of the presently disclosed subject matter.

SEQ ID NO: 27 is the amino acid sequence of the variable heavy chain region of exemplary antibody 6.30 of the presently disclosed subject matter with a C-terminal FuG1 peptide (SEQ ID NO: 44).

SEQ ID NO: 28 is the amino acid sequence of the variable light chain region of exemplary antibody 12.25 of the presently disclosed subject matter.

SEQ ID NOs: 29-31 are the amino acid sequences of the complementarity determining regions (CDRs) 1-3, respectively, of the variable light chain region of exemplary antibody 12.25 of the presently disclosed subject matter.

SEQ ID NO: 32 is the amino acid sequence of the variable heavy chain region of exemplary antibody 6.30 of the presently disclosed subject matter.

SEQ ID NOs: 33-35 are the amino acid sequences of the complementarity determining regions (CDRs) 1-3, respectively, of the variable heavy chain region of exemplary antibody 12.25 of the presently disclosed subject matter.

SEQ ID NO: 36 is the amino acid sequence of the variable heavy chain region of exemplary antibody 12.25 of the presently disclosed subject matter with a C-terminal FuG1 peptide (SEQ ID NO: 44).

SEQ ID NO: 37 is the amino acid sequence of a human IgG4-Fc tagged DR5 construct of the presently disclosed subject matter.

SEQ ID NO: 38 is the amino acid sequence of a random charge control peptide.

SEQ ID NO: 39 is the amino acid sequence of the wild type S1/S2 peptide.

SEQ ID NO: 40 is the amino acid sequence of the RKCR tetrapeptide.

SEQ ID NO: 41 is the amino acid sequence of the RRAR tetrapeptide.

SEQ ID NO: 42 is the amino acid sequence of an RRAR peptide mutant.

SEQ ID NO: 43 is the amino acid sequence of the GSAA tetrapeptide.

SEQ ID NO: 44 is the amino acid sequence of an exemplary FuG1 Fc-Extended Peptide of the presently disclosed subject matter.

SEQ ID NO: 45 is the amino acid sequence of an exemplary cFuG1 Fc-Extended Peptide of the presently disclosed subject matter.

SEQ ID NO: 46 is the amino acid sequence of the variable light chain of exemplary KMTR2 c-kappa light chain.

SEQ ID NO: 47 is the amino acid sequence of the variable heavy chain of exemplary KMTR2 IgG1 heavy chain.

SEQ ID NO: 48 is the amino acid sequence of the variable light chain of exemplary Avelumab c-kappa light chain.

SEQ ID NO: 49 is the amino acid sequence of the variable heavy chain of exemplary Avelumab IgG4 heavy chain.

SEQ ID NO: 50 is the amino acid sequence of the variable light chain of exemplary Farletuzumab c-kappa light chain.

SEQ ID NO: 51 is the amino acid sequence of the variable heavy chain of exemplary Farletuzumab IgG4 heavy chain.

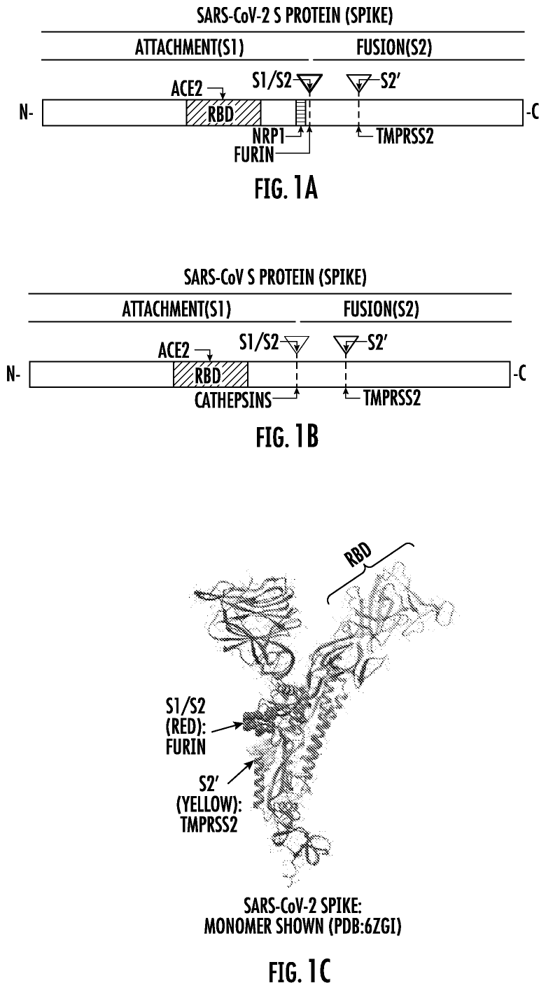

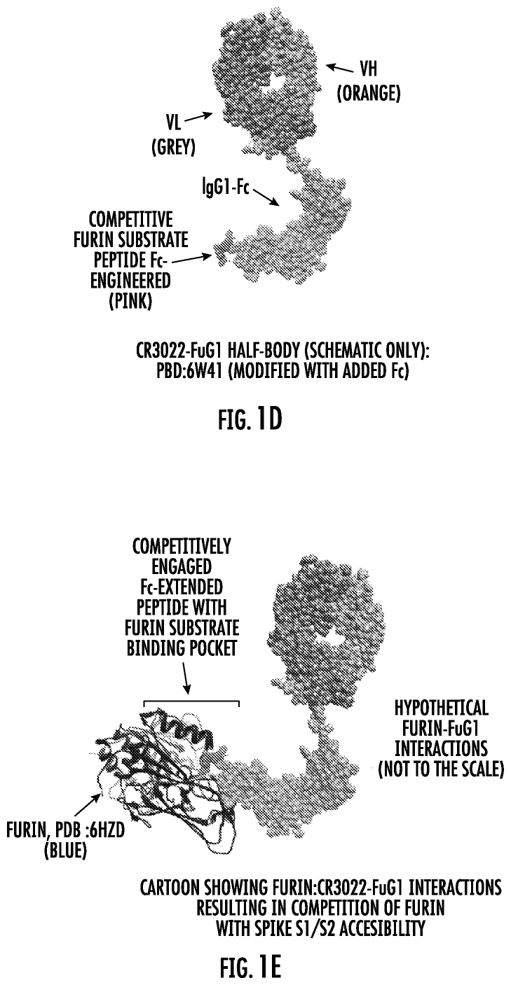

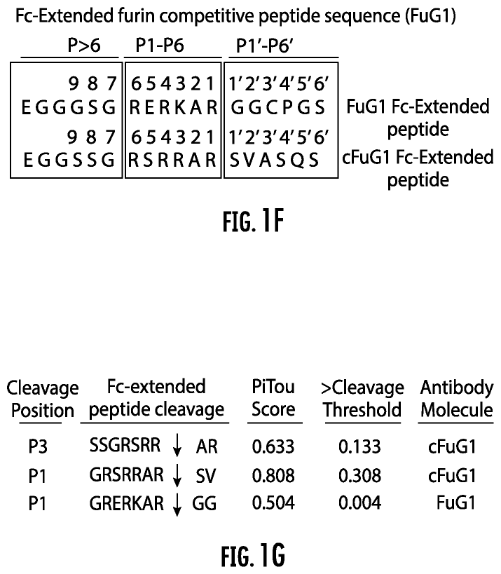

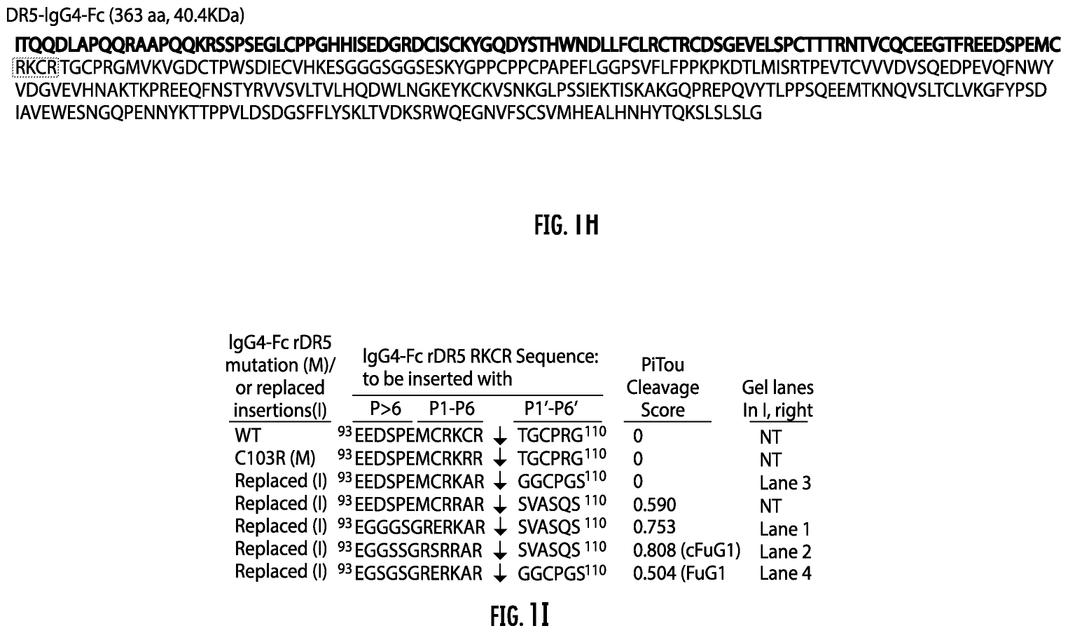

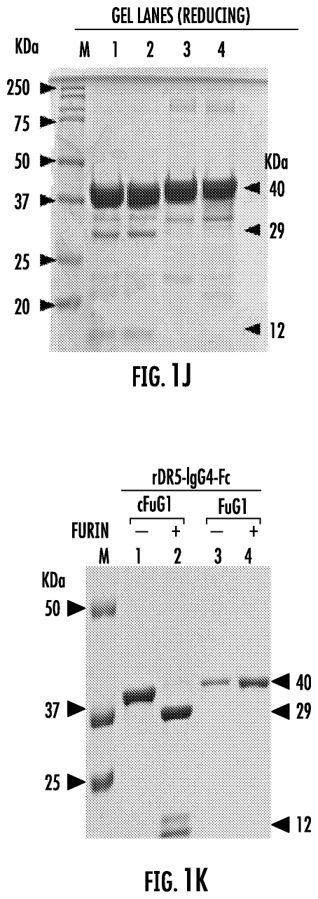

SEQ ID NOs: 52-54 are exemplary amino acids of Fc-extended peptides as shown in FIG. 1G.

SEQ ID NOs: 55-61 are exemplary amino acids of subsequences around the RKCT tetrapeptide of IgG4-Fc rDR5 with modifications as shown in FIG. 1I.

SEQ ID NOs: 62-64 are exemplary amino acids of subsequences of the human IgG4-Fc tagged DR5 construct of the presently disclosed subject matter as shown in FIG. 1H. SEQ ID NO: 65 is the amino acid sequence of a Ravulizumab-42GS FuG1 LINKER RF Hole antibody construct.

SEQ ID NO: 66 is the amino acid sequence of a Eculizumab-42GS FuG1 LINKER RF Hole construct.

### DETAILED DESCRIPTION

### I. General Considerations

The COVID-19 causing coronavirus (SARS-CoV-2) remains a public health threat worldwide. SARS-CoV-2 enters human lung cells via its spike glycoprotein binding to angiotensin-converting enzyme 2 (ACE2). Notably, the cleavage of spike by the host cell protease furin in virus-producing cells is critical for subsequent spike-driven entry into lung cells. Thus, effective targeted therapies blocking the spike cleavage and activation in viral producing cells may provide an alternate strategy to break the viral transmission cycle and to overcome disease pathology. Herein engineered and described is an antibody-based targeted strategy, which directly competes with the furin mediated proteolytic activation of the spike in virus-producing cells. The described approach involves engineering competitive furin substrate residues in the IgG1 Fc-extended flexible linker domain of SARS-CoV-2 spike targeting antibodies. Considering the site of spike furin cleavage and SARS-CoV-2 egress remains uncertain, the experimental strategy pursued here revealed novel mechanistic insights into proteolytic processing of the spike protein, which suggest that processing does not occur in the constitutive secretory pathway. Furthermore, the presently disclosed subject matter shows blockade of furin-mediated cleavage of the spike protein for membrane fusion activation and virus host-cell entry function. These findings provide broad targeting insights of therapeutic applicability to SARS-CoV-2 and the future coronaviridae family members, exploiting the host protease system to gain cellular entry and subsequent pathological infections. Representative sequences in accordance with the presently disclosed subject matter are disclosed in Table 3.

Since its emergence in December 2019, COVID-19 has remained a global economic and health threat. Although RNA and DNA vector-based vaccines induced antibody response and immunological memory have proven highly effective against hospitalization and mortality, their long-term efficacy remains unknown against continuously evolving SARS-CoV-2 variants. As host cell-enriched furin-mediated cleavage of SARS-CoV-2 spike protein is critical for viral entry and chain of the infection cycle, the solution described here of an antibody Fc-conjugated furin competing peptide is significant. In a scenario where spike mutational drifts do not interfere with the Fc-conjugated antibody binding, the presently disclosed furin competing strategy confers a broad-spectrum targeting design to impede the production of efficiently transmissible SARS-CoV-2 viral particles. In addition, the presently disclosed approach is plug-and-play against other viruses, such as but not limited to potentially deadly viruses that exploit secretory pathway independent host protease machinery to gain cellular entry and subsequent transmissions to host cells.

Thus, in some embodiments, the presently disclosed subject matter can be employed for all coronavirus variants, such as SARS-CoV-2 variants (including but not limited to Delta, Omicron, and newly discovered Deltacron). In some embodiments, the virus infection, such as an infection by any coronavirus variant, such as SARS-CoV-2 variants (including but not limited to Delta, Omicron, and newly discovered Deltacron), employs furin cleavage dependent function. The FuG1 Fc peptide sequence strategy in accordance with the presently disclosed subject matter can also be applied to other viruses (including HIV GP160/GP120), in some embodiments such as wherein the surface glycoprotein of virus employs furin cleavage (similar to spike) for activation. The approach is plug and play where antibody spike targeting antibody could be replaced with anti-GP160 and/or anti-GP120 targeting antibodies for HIV. HIV is similar in that RBD or S1 targeting antibody would not only bind spike S1 but Spike S0, too. Exemplary HIV targeting antibodies are known in the art and include but are not limited to GP120 targeting antibodies, which also targets GP160 (as GP120 is part of GP160), such as but not limited to those disclosed in PCT International Patent Application Publication No. WO 2020/010107, published Jan. 9, 2020, herein incorporated by reference in its entirety.

Representative farletuzumab, KMTR2, and avelumab VH and VL sequences are described herein, including in Table 3. However, the heavy chain (VH) of these were modified in the end of their VH sequence after the final Gly (G), Lys (K) residues to add the FuG1 peptide (also referred to as the FuG1 Fc-extended peptide): EGGGSGRERKARGGCPGS (SEQ ID NO: 44).

Additional exemplary antibodies and reagents are disclosed in PCT International Patent Application Publication No. WO 2021/150770, published Jul. 29, 2021, herein incorporated by reference in its entirety, and PCT International Patent Application Publication No. WO 2019/099374, published May 23, 2019, herein incorporated by reference in its entirety.

### II. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of”′ and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

As use herein, the terms “administration of” and or “administering” a compound should be understood to mean providing a compound of the presently disclosed subject matter or a prodrug of a compound of the presently disclosed subject matter to a subject in need of treatment.

The term “adult” as used herein, is meant to refer to any non-embryonic or non-juvenile subject. For example, the term “adult adipose tissue stem cell”, refers to an adipose stem cell, other than that obtained from an embryo or juvenile subject.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom”, means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, and/or by the one-letter code corresponding thereto, as summarized in the following Table 1:

TABLE 1
Amino Acid Codes and Functionally Equivalent Codons

| | 3-Letter | 1-Letter | Functionally |
|---|---|---|---|
| Full Name | Code | Code | Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; |
| | | | CGC; CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; |
| | | | UCC; UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; |
| | | | CUC; CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is melant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

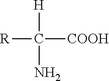

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fv, single chain Fv (scFv), complementarity determining regions (CDRs), VL (light chain variable region), VH (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to VH—CH1 by a disulfide bond. The F(ab′)₂ may be reduced under mild conditions to break the disulfide linkage in the hinge region, thereby converting the F(ab′)₂ dimer into an Fab₁ monomer. The Fab₁ monomer is essentially an Fab with part of the hinge region (see Paul, 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al., 1988, both of which are incorporated by reference herein. For a review article concerning humanized antibodies, see Winter & Milstein, 1991, incorporated by reference herein. See also U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically competent cells, or both. An antigen can be derived from organisms, subunits of proteins/antigens, killed or inactivated whole cells or lysates.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to reactions as described herein. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

As used herein, the term “conservative amino acid substitution” is defined herein as an amino acid exchange within one of the five groups summarized in the following Table 2:

TABLE 2
Conservative Amino Acid Substitutions

| Group | Characteristics | Amino Acids |
|---|---|---|
| A. | Small aliphatic, nonpolar, | Ala, Ser, Thr, Pro, Gly |
| | or slightly polar residues | |
| B. | Polar, negatively charged | Asp, Asn, Glu, Gln |
| | residues and their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is cancer, which in some embodiments comprises a solid tumor.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length. In the case of a shorter sequence, fragments are shorter.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993). This algorithm is incorporated into the NBLAST and XBLAST programs of Altschul et al., 1990a and Althschul et al., 1990b, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

Used interchangeably herein are the terms: 1) “isolate” and “select”; and 2) “detect” and “identify”.

The term “isolated”, when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically or selectively binds to a target compound. A ligand (e.g., an antibody) “specifically binds to”, “is specifically immunoreactive with”, “having a selective binding activity”, “selectively binds to” or “is selectively immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically or selectively binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988, for a description of immunoassay formats and conditions that can be used to determine specific immunoreactivity.

A “receptor” is a compound that specifically or selectively binds to a ligand.

A ligand or a receptor (e.g., an antibody) “specifically binds to”, “is specifically immunoreactive with”, “having a selective binding activity”, “selectively binds to” or “is selectively immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically or selectively binds under hybridization conditions to a compound polynucleotide comprising a complementary sequence; an antibody specifically or selectively binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane, 1988 for a description of immunoassay formats and conditions that can be used to determine specific or selective immunoreactivity. See also the EXAMPLES set forth herein below for additional formats and conditions that can be used to determine specific or selective immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder, such as, but not limited to, in a subject that has been exposed to a pathogen, who is at risk for exposure to a pathogen, and/or who would be particularly susceptible to suffering from severe disease if exposed to a pathogen or after exposure to a pathogen.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases scFv are expressed on the surface of an engineered cell, for the purpose of selecting particular scFv that bind to an antigen of interest.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

In some embodiments, a “homolog” is a polypeptide that has a substantially homologous amino acid sequence to another reference amino acid sequence. As such, and without limitation, in some embodiments, a “homolog” of a reference amino acid sequence is a second amino acid sequence that is at least 95% identical to said reference amino acid sequence, in some embodiments over the full length of one or both amino acid sequences.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and more in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter. In some embodiments, the subject in need of treatment is a mammal. In some embodiments, the subject is a human. In some embodiments, the subject is a subject who has a SARS-CoV-2 infection or another virus infection; a subject who is suspected of having a SARS-CoV-2 infection or another virus infection (e.g., by exhibiting one or more symptoms of a SARS-CoV-2 infection or another virus infection); or who is a subject who has one or more increased risk factors for a SARS-CoV-2 infection or another virus infection. Increased risk factors for getting a SARS-CoV-2 infection or another virus infection include, but are not limited to, contact with another who has tested positive for SARS-CoV-2 infection or another virus infection and/or contact with someone who has exhibited symptoms of SARS-CoV-2 infection or another virus infection. Symptoms and increased risk factors include, but are not limited to, fever, chills, cough, difficulty breathing, headache, fatigue, sore throat, muscle or body ache, loss of smell or taste, nausea, and diarrhea; living in communal housing (e.g., a nursing home); and travel to areas with high rates of SARS-CoV-2 infectivity or high rates of infectivity of another virus. In some embodiments, the subject is a subject who has increased risk for severe COVID-19 or for a severe form of another viral disease (e.g., risk for disease requiring hospitalization, ventilation or oxygen support, and/or that results in death). Subjects with increased risk of severe disease include human subjects over the age of 45 or over the age of 65. Underlying medical conditions or comorbidities that can result in higher risk of severe disease (e.g., of severe COVID-19) include, but are not limited to, cancer, chronic kidney disease, chronic lung disease (e.g., chronic obstructive pulmonary disease (COPD), asthma, cystic fibrosis, pulmonary hypertension, and interstitial lung disease), dementia, diabetes, Down's syndrome, heart disease (e.g., heart failure, coronary artery disease, hypertension, etc.), HIV, a compromised or weakened immune system, liver disease (e.g., cirrhosis), being overweight (i.e., having a body mass index (BMI)>25 kg/m²) or obese (i.e., having a BMI≥30 kg/m²), pregnancy, sickle cell disease or thalassemia, a history of smoking, stroke, and having a history of substance abuse.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

One of ordinary skill in the art will appreciate that based on the sequences of the components of the antibodies disclosed herein they can be modified independently of one another with conservative amino acid changes, including, insertions, deletions, and substitutions, and that the valency could be altered as well. Amino acid changes (fragments and homologs) can be made independently in an antibody as well when they are being used in a therapy.

The presently disclosed subject matter provides other antibodies and biologically active fragments and homologs thereof as well as methods for preparing and testing new antibodies for the properties disclosed herein.

In some embodiments, the fragments are fragments of scFv. In some embodiments, the scFv fragments are mammalian. In some embodiments, the scFv fragments are humanized.

In some embodiments, the presently disclosed subject matter uses a biologically active antibody or biologically active fragment or homolog thereof. In some embodiments, the isolated polypeptide comprises a mammalian molecule at least about 30% homologous to a polypeptide having the amino acid sequence of at least one of the sequences disclosed herein. In some embodiments, the isolated polypeptide is at least about 35% homologous, more in some embodiments, about 40% homologous, more in some embodiments, about 45% homologous, in some embodiments, about 50% homologous, more in some embodiments, about 55% homologous, in some embodiments, about 60% homologous, more in some embodiments, about 65% homologous, in some embodiments, more in some embodiments, about 70% homologous, more in some embodiments, about 75% homologous, in some embodiments, about 80% homologous, more in some embodiments, about 85% homologous, more in some embodiments, about 90% homologous, in some embodiments, about 95% homologous, more in some embodiments, about 96% homologous, more in some embodiments, about 97% homologous, more in some embodiments, about 98% homologous, and most in some embodiments, about 99% homologous to at least one of the peptide sequences disclosed herein.

The presently disclosed subject matter further encompasses modification of the antibodies and fragments thereof disclosed herein, including amino acid deletions, additions, and substitutions, particularly conservative substitutions. The presently disclosed subject matter also encompasses modifications to increase in vivo half-life and decrease degradation in vivo. Substitutions, additions, and deletions can include, for example, 1, 2, 3, 4, 5, 6, 7, 8, 9, 10, 11, 12, 13, 14, 15, 16, 17, 18, 19, 20, 21, 22, 23, 24, and 25 changes as long as the activity disclosed herein remains substantially the same.

The presently disclosed subject matter includes an isolated nucleic acid comprising a nucleic acid sequence encoding an antibody of the presently disclosed subject matter, or a fragment or homolog thereof. In some embodiments, the nucleic acid sequence encodes a peptide comprising an antibody sequence of the presently disclosed subject matter, or a biologically active fragment of homolog thereof.

In some embodiments, a homolog of a peptide (antibody or fragment) of the presently disclosed subject matter is one with one or more amino acid substitutions, deletions, or additions, and with the sequence identities described herein. In some embodiments, the substitution, deletion, or addition is conservative.

In some embodiments, the subject is a mammal. In some embodiments, the mammal is a human.

The presently disclosed subject matter encompasses the use of purified isolated, recombinant, and synthetic peptides.

### III Representative Methods and Compositions

In some embodiments, the presently disclosed subject matter provides a method for treating a viral infection in a subject in need thereof. In some embodiments, the method comprises administering to the subject a composition comprising an effective amount of an agent that selectively interferes with host protease function to inhibit fusion-ready viral fragment generation (such as but not limited to S2 in case of SARS-CoV2 or GP160 or GP120 in case of HIV) and/or to destabilize a full-length viral fusion protein (such as but not limited to spike).

In some embodiments, the presently disclosed subject matter provides a composition comprising an effective amount of an agent that selectively interferes with host protease function to inhibit fusion-ready viral fragment generation (such as but not limited to S2 in case of SARS-CoV2 or GP160 or GP120 in case of HIV) and/or to destabilize a full-length viral fusion protein (such as but not limited to spike). The composition can be for use in treating a viral infection. In some embodiments, the presently disclosed subject matter provides for the treatment of COVID19, including long COVID19.

In some embodiments, the composition comprises an effective amount of an agent that selectively interferes with host furin protease function to inhibit fusion-ready S2 fragment generation and/or to destabilize full-length spike (S0) protein.

In some embodiments, the viral infection is a HIV infection or a coronavirus infection, such as but not limited to Delta variant, Omicron variant, or Deltacron variant. In some embodiments, the viral infection is a SAR-CoV-2 infection, such as but not limited to Delta variant, Omicron variant, or Deltacron variant.

In some embodiments, the agent comprises an Fc-conjugated furin competitive peptide and an antibody. In some embodiments, the agent comprises a sequence as set forth in Table 3, or a biologically active fragment and/or homolog thereof. In some embodiments, the Fc-conjugated peptide comprises a sequence as set forth in Table 3 or a biologically active fragment and/or homolog thereof.

In some embodiments, the agent comprises an Fc-conjugated furin competitive peptide and an antibody, and the Fc-conjugated furin competitive peptide is uncleavable by furin. In some embodiments, the agent comprises an Fc-conjugated furin competitive peptide and an antibody, wherein the Fc-conjugated furin competitive peptide is stable, flexible, and can be conjugated to any antibody targeting a virus.

In some embodiments, the agent comprises an Fc-conjugated furin competitive peptide and an antibody, wherein the antibody Fc-conjugated furin competitive peptide is against furin recognition sequence of gp160 or gp120 of HIV or spike of SARS-CoV-2. Representative non-limiting embodiments are further disclosed in Examples 1-9 as set forth herein below. By way of particular example, KMTR2 is a DR5 antibody. A FuG1 peptide is added to KMTR2 Fc to show that adding the FuG1 peptide does not change other binding function of antibodies. This shows the plug and play aspects of the presently disclosed subject matter. In some embodiments, disclosed is fusing the FuG1 peptide to the Fc of anti-C5 (a complement component) antibody. It has been shown that overactivation of complement system in human body plays a role in Long COVID. Thus, in some embodiments, proposed is targeting SARS-CoV2 near complement activating cells. In terms of antibodies, ravulizumab and eculizumab can be used for adding FuG1 peptide. The sequences of ravulizumab and eculizumab is available at the website of the RCSB Protein Data Bank (rcsb.org), and exemplary amino acid sequences are presented as SEQ ID NOs: 65 and 66, respectively. See also Ladwig & Willrich, 2021.

In some embodiments, the agent comprises a bispecific or other multispecific antibody, such that multiple epitopes in the subject can be targeted. For example, antibody having complement function interfering and spike-directed furin cleavage blocking functionality are provided. Representative antibody sequences are presented in Table 3. In some embodiments, the antibody comprises an antigen binding site specific for a first epitope and an antigen binding site specific for a second epitope. In some embodiments, the antigen binding site specific for said first epitope is at the amino terminus end of the variable region. In some embodiments, the antigen binding site specific for the second epitope is linked to the carboxy end of the CH3 constant region. In some embodiments, the binding affinity of the first epitope to the antigen binding site specific for the first epitope and the binding affinity of the second epitope to the antigen binding site specific for the second epitope are unchanged after conversion of the antigen binding sites into a bispecific configuration. In some embodiments, the bispecific antibody comprises a heavy chain of SEQ ID NO: 51 and a light chain of SEQ ID NO: 50, or biologically active fragments and homologs thereof, wherein SEQ ID NO: 51 is a heavy chain variable amino acid sequence of Farletuzumab and SEQ ID NO: 50 is a light chain variable amino acid sequence of Farletuzumab. Representative non-limiting embodiments are further disclosed in EXAMPLES 1-9 as set forth herein below.

By way of particular example, KMTR2 is a DR5 antibody. A FuG1 peptide is added to KMTR2 Fc to show that adding the FuG1 peptide does not change other binding function of antibodies. This shows the plug and play aspects of the presently disclosed subject matter. In some embodiments, disclosed is fusing the FuG1 peptide to the Fc of anti-C5 (a complement component) antibody. It has been shown that overactivation of complement system in human body plays a role in Long COVID. Thus, in some embodiments, proposed is targeting SARS-CoV2 near complement activating cells. In terms of antibodies, ravulizumab and eculizumab can be used for adding FuG1 peptide. The sequence of ravulizumab and eculizumab is available at the website of the RCSB Protein Data Bank (rcsb.org). See also Ladwig & Willrich, 2021.

Thus, in some embodiments, an agent of the presently disclosed subject matter comprises an antibody comprising a complement component antibody or a biologically active fragment and/or homolog thereof. A representative complement component is C5, but any suitable complement component as would be apparent to one of ordinary skill in the art upon a review of can be targeted. In some embodiments, the complement component antibody comprises an anti C5 antibody or a biologically active fragment and/or homolog thereof. In some embodiments, the complement component antibody comprises ravulizumab and eculizumab, or a biologically active fragment and/or homolog thereof. In some embodiments, the complement component antibody comprises SEQ ID NO: 65 and/or and SEQ ID NO: 66, or a biologically active fragment and/or homolog thereof, such as a sequence at least 95% identical thereto.

In some embodiments, the antibodies are humanized. In some embodiments, the composition further comprises a pharmaceutically acceptable carrier. In some embodiments, the composition is for use in in treating a viral infection. In some embodiments, the viral infection is a HIV infection or a coronavirus infection, such as but not limited to Delta variant, Omicron variant, or Deltacron variant. In some embodiments, the viral infection is a SAR-CoV-2 infection, such as but not limited to Delta variant, Omicron variant, or Deltacron variant. In some embodiments, the composition is for use in treating COVID19, including long COVID19. In some embodiments, an additional therapeutic agent is administered.

### Peptide Modification and Preparation

It will be appreciated, of course, that the proteins or peptides of the presently disclosed subject matter may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical, or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C1-C5 branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the presently disclosed subject matter are also contemplated as functional equivalents. Thus, a peptide in accordance with the presently disclosed subject matter treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the presently disclosed subject matter.

The presently disclosed subject matter also provides for analogs of proteins, e.g., analogs of antibodies. Analogs can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. To that end, 10 or more conservative amino acid changes typically have no effect on peptide function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring or non-standard synthetic amino acids. The peptides of the presently disclosed subject matter are not limited to products of any of the specific exemplary processes listed herein.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical, or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C1-C5 branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH2), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

Substantially pure protein obtained as described herein may be purified by following known procedures for protein purification, wherein an immunological, enzymatic, or other assay is used to monitor purification at each stage in the procedure. Protein purification methods are well known in the art, and are described, for example in Deutscher et al., 1990.

As discussed, modifications or optimizations of peptide ligands of the presently disclosed subject matter are within the scope of the application. Modified or optimized peptides are included within the definition of peptide binding ligand. Specifically, a peptide sequence identified can be modified to optimize its potency, pharmacokinetic behavior, stability and/or other biological, physical, and chemical properties.

### Amino Acid Substitutions

In certain embodiments, the disclosed methods and compositions may involve preparing peptides with one or more substituted amino acid residues.

In various embodiments, the structural, physical and/or therapeutic characteristics of peptide sequences may be optimized by replacing one or more amino acid residues.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C1-10 carbons including branched, cyclic, and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3- or 4-chlorophenylalanine, 2-,3- or 4-methylphenylalanine, 2-,3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′, -3′, or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C1-C10 branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, amino acids whose hydropathic indices are in some embodiments within +/−2 can be employed, in some embodiments within +/−1 can be employed, and in some embodiments within +/−0.5 can be employed.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see e.g., Chou & Fasman, 1974; Chou & Fasman, 1978; Chou & Fasman, 1979).

Based on such considerations and extensive empirical study, tables of conservative amino acid substitutions have been constructed and are known in the art. For example: arginine and lysine; glutamate and aspartate; serine and threonine; glutamine and asparagine; and valine, leucine, and isoleucine. Alternatively, Ala (A): Leu, Ile, Val; Arg (R): Gln, Asn, Lys; Asn (N): His, Asp, Lys, Arg, Gln; Asp (D): Asn, Glu; Cys (C): Ala, Ser; Gln (Q): Glu, Asn; Glu (E): Gln, Asp; Gly (G): Ala; His (H): Asn, Gln, Lys, Arg; Ile (I): Val, Met, Ala, Phe, Leu; Leu (L): Val, Met, Ala, Phe, Ile; Lys (K): Gln, Asn, Arg; Met (M): phe, Ile, Leu; Phe (F): Leu, Val, Ile, Ala, Tyr; Pro (P): Ala; Ser(S): Thr; Thr (T): Ser; Trp (W): Phe, Tyr; Tyr (Y): Trp, Phe, Thr, Ser; Val (V): Ile, Leu, Met, Phe, Ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. See e.g., the PROWL Rockefeller University website. For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix, and Risler matrix.

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

### Antibody Formats and Preparation Thereof

Antibodies directed against proteins, polypeptides, or peptide fragments thereof of the presently disclosed subject matter may be generated using methods that are well known in the art. For instance, U.S. Pat. No. 5,436,157, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

In some embodiments, one or more antibodies or fragments thereof are used. In some embodiments, one or both antibodies are single chain, monoclonal, bi-specific, synthetic, polyclonal, chimeric, human, or humanized, or active fragments or homologs thereof. In some embodiments, the antibody binding fragment is scFV, F(ab′)₂, F(ab)₂, Fab′, or Fab.

For the preparation of monoclonal antibodies, any technique which provides for the production of antibody molecules by continuous cell lines in culture may be utilized. For example, the hybridoma technique originally developed by Kohler & Milstein (Kohler & Milstein, 1975), the trioma technique, the human B-cell hybridoma technique (Kozbor & Roder, 1983), and the EBV-hybridoma technique (Cole et al., 1985) may be employed to produce human monoclonal antibodies. In some embodiments, monoclonal antibodies are produced in germ-free animals.

In accordance with the presently disclosed subject matter, human antibodies may be used and obtained by utilizing human hybridomas (Cote et al., 1983) or by transforming human B cells with EBV virus in vitro (Cole et al., 1985). Furthermore, techniques developed for the production of “chimeric antibodies” (Morrison et al., 1984; Neuberger et al., 1984; Takeda et al., 1985) by splicing the genes from a mouse antibody molecule specific for epitopes of SLLP polypeptides together with genes from a human antibody molecule of appropriate biological activity can be employed; such antibodies are within the scope of the presently disclosed subject matter. Once specific monoclonal antibodies have been developed, the preparation of mutants and variants thereof by conventional techniques is also available.

Various techniques have been developed for the production of antibody fragments of humanized antibodies. Traditionally, these fragments were derived via proteolytic digestion of full-length antibodies (see e.g., Morimoto & Inouye, 1992; Brennan et al., 1985). However, these fragments can now be produced directly by recombinant host cells. Alternatively, Fab′-SH fragments can be directly recovered from E. coli and chemically coupled to form F(ab′) 2 fragments (Carter et al., 1992a). According to another approach, F(ab′) 2 fragments can be isolated directly from recombinant host cell culture. Other techniques for the production of antibody fragments will be apparent to the skilled practitioner. In other embodiments, the antibody of choice is a single-chain Fv fragment (scFv). See PCT International Patent Application Publication No. WO 1993/16185; U.S. Pat. Nos. 5,571,894; 5,587,458. The antibody fragment may also be a “linear antibody”, e.g., as described in U.S. Pat. No. 5,641,870, for example. Such linear antibody fragments may be monospecific or bispecific.

Humanized (chimeric) antibodies are immunoglobulin molecules comprising a human and non-human portion. More specifically, the antigen combining region (or variable region) of a humanized chimeric antibody is derived from a non-human source (e.g., murine) and the constant region of the chimeric antibody (which confers biological effector function to the immunoglobulin) is derived from a human source. The humanized chimeric antibody should have the antigen binding specificity of the non-human antibody molecule and the effector function conferred by the human antibody molecule. A large number of methods of generating chimeric antibodies are well known to those of skill in the art (see e.g., U.S. Pat. Nos. 4,975,369; 5,075,431; 5,081,235; 5,169,939; 5,202,238; 5,204,244; 5,231,026; 5,292,867; 5,354,847; 5,472,693; 5,482,856; 5,491,088; 5,500,362; and 5,502,167). Detailed methods for preparation of chimeric (humanized) antibodies can be found in U.S. Pat. No. 5,482,856. A “humanized” antibody is a human/non-human chimeric antibody that contains a minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody can optionally also comprise at least a portion of an immunoglobulin constant region (Fc), typically that of a human immunoglobulin. For further details, see e.g., Jones et al., 1986; Riechmann et al., 1988; Presta, 1992, PCT International Patent Application Publication No. WO 92/02190, U.S. Patent Application Publication No. 2006/0073137, and U.S. Pat. Nos. 5,225,539; 5,530,101; 5,585,089; 5,693,761; 5,693,762; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,548,640; 6,632,927; 6,639,055; and 6,750,325.

In some embodiments, the presently disclosed subject matter provides for fully human antibodies. Human antibodies consist entirely of characteristically human polypeptide sequences. The human antibodies of this presently disclosed subject matter can be produced in using a wide variety of methods (see e.g., U.S. Pat. No. 5,001,065, for review).

Typically, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al, 1986; Riechmann et al., 1988; Verhoeyen et al., 1988), by substituting hypervariable region sequences for the corresponding sequences of a human “acceptor” antibody. Accordingly, such “humanized” antibodies are chimeric antibodies (see e.g., U.S. Pat. Nos. 4,816,567 and 5,482,856) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

Another method for making humanized antibodies is described in U.S. Patent Application Publication No. 2003/0017534, wherein humanized antibodies and antibody preparations are produced from transgenic non-human animals. The non-human animals are genetically engineered to contain one or more humanized immunoglobulin loci that are capable of undergoing gene rearrangement and gene conversion in the transgenic non-human animals to produce diversified humanized immunoglobulins.

In some embodiments, the choice of human variable domains, both light and heavy, to be used in making the humanized antibodies is very important to reduce antigenicity. According to the so-called “best-fit” method, the sequence of the variable domain of a rodent antibody is screened against a library of known human variable-domain sequences or a library of human germline sequences. The human sequence that is closest to that of the rodent can then be accepted as the human framework region for the humanized antibody (Sims et al., 1993; Chothia & Lesk, 1987). Another method uses a particular framework region derived from the consensus sequence of all human antibodies of a particular subgroup of light or heavy chains. The same framework may be used for several different humanized antibodies (Carter et al., 1992b; Presta et al., 1993). Other methods designed to reduce the immunogenicity of the antibody molecule in a human patient include veneered antibodies (see e.g., U.S. Pat. No. 6,797,492 and U.S. Patent Application Publication Nos. 2002/0034765 and 2004/0253645) and antibodies that have been modified by T-cell epitope analysis and removal (see e.g., U.S. Patent Application Publication No. 2003/0153043 and U.S. Pat. No. 5,712,120).

It is important that when antibodies are humanized they retain high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

The antibody moieties of this presently disclosed subject matter can be single chain antibodies.

Antibodies directed against proteins, polypeptides, or peptide fragments thereof of the presently disclosed subject matter may be generated using methods that are well known in the art. For instance, U.S. Pat. No. 5,436,157, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

The hybrid antibodies and hybrid antibody fragments include complete antibody molecules having full length heavy and light chains, or any fragment thereof, such as Fab, Fab′, F(ab′)₂, Fd, scFv, antibody light chains and antibody heavy chains. Chimeric antibodies which have variable regions as described herein and constant regions from various species are also suitable. See for example, U.S. Patent Application No. 2003/0022244.

Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, F(ab′)₂, and single chain Fv (scFv) fragments.

In some embodiments, the specific binding molecule is a single-chain variable analogue (scFv). The specific binding molecule or scFv may be linked to other specific binding molecules (for example other scFvs, Fab antibody fragments, chimeric IgG antibodies (e.g., with human frameworks)) or linked to other scFvs of the presently disclosed subject matter so as to form a multimer which is a multi-specific binding protein, for example a dimer, a trimer, or a tetramer. Bi-specific scFvs are sometimes referred to as diabodies, tri-specific such as triabodies and tetra-specific such as tetrabodies when each scFv in the dimer, trimer, or tetramer has a different specificity. Diabodies, triabodies, and tetrabodies can also be monospecific, when each scFv in the dimer, trimer, or tetramer has the same specificity.

In some embodiments, techniques described for the production of single-chain antibodies (U.S. Pat. No. 4,946,778, incorporated by reference herein in its entirety) are adapted to produce protein-specific single-chain antibodies. In some embodiments, the techniques described for the construction of Fab expression libraries (Huse et al., 1989) are utilized to allow rapid and easy identification of monoclonal Fab fragments possessing the desired specificity for specific antigens, proteins, derivatives, or analogs of the presently disclosed subject matter.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which bind the antigen therefrom at any epitopes present therein.

Monoclonal antibodies directed against full length or peptide fragments of a protein or peptide may be prepared using any well known monoclonal antibody preparation procedures, such as those described, for example, in Harlow & Lane, 1988; Tuszynski et al., 1988). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Exemplary complementarity-determining region (CDR) residues or sequences and/or sites for amino acid substitutions in framework region (FR) of such humanized antibodies having improved properties such as, e.g., lower immunogenicity, improved antigen-binding or other functional properties, and/or improved physicochemical properties such as, e.g., better stability, are provided.

The presently disclosed subject matter encompasses more than the specific fragments and humanized fragments disclosed herein. In some embodiments, the antibody is selected from the group consisting of a single chain antibody, a monoclonal antibody, a bi-specific antibody, a chimeric antibody, a synthetic antibody, a polyclonal antibody, or a humanized antibody, or active fragments or homologs thereof.

A nucleic acid encoding the monoclonal antibody obtained using the procedures described herein may be cloned and sequenced using technology which is available in the art, and is described, for example, in Wright et al., 1992) and the references cited therein. Further, the antibody of the presently disclosed subject matter may be “humanized” using the technology described in Wright et al., 1992 and in the references cited therein, and in Gu et al., 1997.

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes.

The procedures for making a bacteriophage library comprising heterologous DNA are well known in the art and are described, for example, in Green & Sambrook, 2012.

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above, have been developed for the production of human antibodies using M13 bacteriophage display (Burton & Barbas, 1994). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

In accordance with the presently disclosed subject matter, human antibodies may be used and obtained by utilizing human hybridomas (Cote et al., 1983) or by transforming human B cells with EBV virus in vitro (Cole et al., 1985). Furthermore, techniques developed for the production of “chimeric antibodies” (Morrison et al., 1984; Neuberger et al., 1984; Takeda et al., 1985).

The procedures just presented describe the generation of phage which encode the Fab portion of an antibody molecule. However, the presently disclosed subject matter should not be construed to be limited solely to the generation of phage encoding Fab antibodies. Rather, phage which encode single chain antibodies (scFv/phage antibody libraries) are also included in the presently disclosed subject matter. Fab molecules comprise the entire Ig light chain, that is, they comprise both the variable and constant region of the light chain, but include only the variable region and first constant region domain (CH₁) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991. Panning of phage so generated for the isolation of a desired antibody is conducted in a manner similar to that described for phage libraries comprising Fab DNA.

The presently disclosed subject matter should also be construed to include synthetic phage display libraries in which the heavy and light chain variable regions may be synthesized such that they include nearly all possible specificities (Barbas, 1995; de Kruif et al., 1995).

In the production of antibodies, screening for the desired antibody can be accomplished by techniques known in the art, e.g., ELISA (enzyme-linked immunosorbent assay). Antibodies generated in accordance with the presently disclosed subject matter may include, but are not limited to, polyclonal, monoclonal, chimeric (i.e., “humanized”), and single chain (recombinant) antibodies, Fab fragments, and fragments produced by a Fab expression library.

Substantially pure peptide obtained as described herein may be purified by following known procedures for protein purification, wherein an immunological, enzymatic, or other assay is used to monitor purification at each stage in the procedure. Protein purification methods are well known in the art, and are described, for example in Deutscher et al., 1990.

It is common in the field of recombinant humanized antibodies to graft murine CDR sequences onto a well-established human immunoglobulin framework previously used in human therapies such as the framework regions of Herceptin [Trastuzumab].

In some embodiments, when used in vivo for therapy, the antibodies of the subject presently disclosed subject matter are administered to the subject in therapeutically effective amounts (i.e., amounts that have desired therapeutic effect). They will normally be administered parenterally. The dose and dosage regimen will depend upon the degree of the infection, the characteristics of the particular antibody or immunotoxin used, e.g., its therapeutic index, the patient, and the patient's history. Advantageously the antibody or immunotoxin is administered continuously over a period of 1-2 weeks. Optionally, the administration is made during the course of adjunct therapy such as antimicrobial treatment, or administration of tumor necrosis factor, interferon, or other cytoprotective or immunomodulatory agent.

In some embodiments, for parenteral administration, the antibodies will be formulated in a unit dosage injectable form (solution, suspension, emulsion) in association with a pharmaceutically acceptable parenteral vehicle. Such vehicles are inherently nontoxic, and non-therapeutic. Examples of such vehicle are water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability, e.g., buffers and preservatives. The antibodies will typically be formulated in such vehicles at concentrations of about 1.0 mg/ml to about 10 mg/ml.

### Pharmaceutical Compositions and Administration

The presently disclosed subject matter is also directed to methods of administering the compounds of the presently disclosed subject matter to a subject.

Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

In accordance with one embodiment, a method of treating a subject in need of such treatment is provided. The method comprises administering a pharmaceutical composition comprising at least one compound of the presently disclosed subject matter to a subject in need thereof. Compounds identified by the methods of the presently disclosed subject matter can be administered with known compounds or other medications as well.

The pharmaceutical compositions useful for practicing the presently disclosed subject matter may be administered to deliver a dose of between 1 ng/kg/day and 100 mg/kg/day.

The presently disclosed subject matter encompasses the preparation and use of pharmaceutical compositions comprising a compound useful for treatment of the diseases and disorders disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter may comprise at least one active peptide, one or more acceptable carriers, and optionally other peptides or therapeutic agents.

For in vivo applications, the peptides of the presently disclosed subject matter may comprise a pharmaceutically acceptable salt. Suitable acids which are capable of forming such salts with the compounds of the presently disclosed subject matter include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The peptides of the presently disclosed subject matter, pharmaceutically acceptable salts thereof, or pharmaceutical compositions comprising these compounds may be administered so that the compounds may have a physiological effect. Administration may occur enterally or parenterally; for example, orally, rectally, intracisternally, intravaginally, intraperitoneally, locally (e.g., with powders, ointments or drops), or as a buccal or nasal spray or aerosol. Parenteral administration is preferred. Particularly preferred parenteral administration methods include intravascular administration (e.g., intravenous bolus injection, intravenous infusion, intra-arterial bolus injection, intra-arterial infusion and catheter instillation into the vasculature), peri- and intra-target tissue injection (e.g., peri-tumoral and intra-tumoral injection), subcutaneous injection or deposition including subcutaneous infusion (such as by osmotic pumps), intramuscular injection, and direct application to the target area, for example by a catheter or other placement device.

Where the administration of the peptide is by injection or direct application, the injection or direct application may be in a single dose or in multiple doses. Where the administration of the compound is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Genaro, 1985, which is incorporated herein by reference.

Typically, dosages of the compound of the presently disclosed subject matter which may be administered to an animal, in some embodiments a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In another aspect, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes administering the composition to a subject. In some embodiments, this kit comprises a (in some embodiments sterile) solvent suitable for dissolving or suspending the composition of the presently disclosed subject matter prior to administering the compound to the subject.

As used herein, an “instructional material” includes a publication, a recording, a diagram, or any other medium of expression which can be used to communicate the usefulness of a composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains a composition of of the presently disclosed subject matter or be shipped together with a container which contains the composition. Alternatively, the instructional material may be shipped separately from the container with the intention that the instructional material and the compound be used cooperatively by the recipient.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Materials and Methods Employed in the Examples

Source of antibody sequences. The sequence of anti-SARS-CoV spike RBD (RBD-C) targeting immunoglobulin heavy chain and light chain are available at Accession Numbers DQ168569.1 (CR3022 VH) and DQ168570.1 (CR3022 VL) of the GENBANK® biosequence database. The sequences of anti-SARS-CoV spike (outside RBD, named S—B) targeting immunoglobulin heavy chain and light chain are available at Accession Nos. MT594062.1 (12.25 VH) and MT594095.1 (12.25 VL) of the GENBANK® biosequence database. These antibodies are described in Rogers et al., 2020. The sequences of farletuzumab, KMTR2, etc. used are described in Shivange et al., 2018 and are publicly available at IMTG.org.

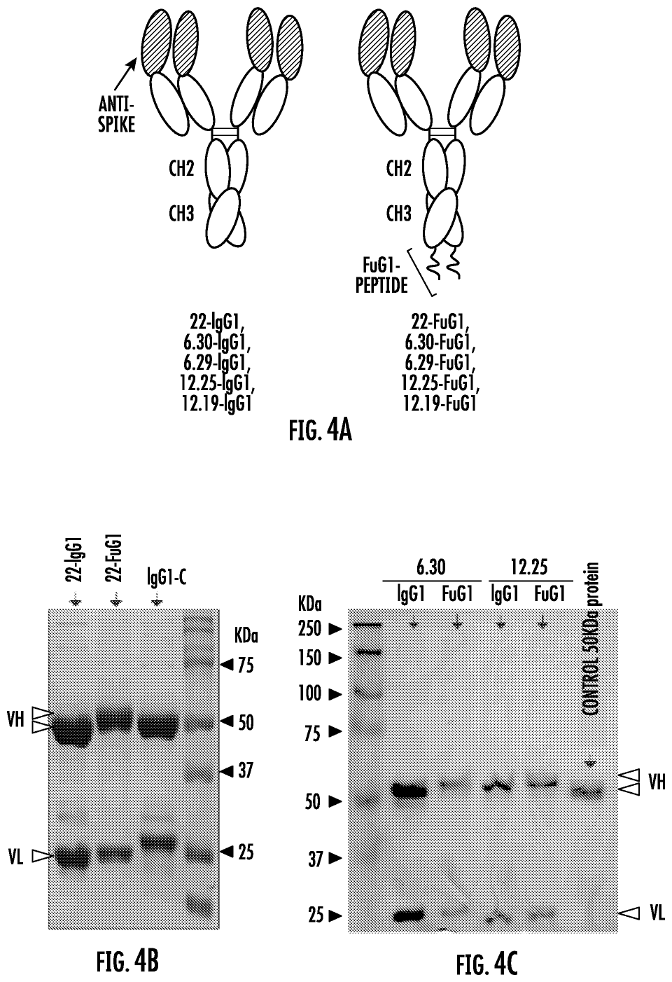

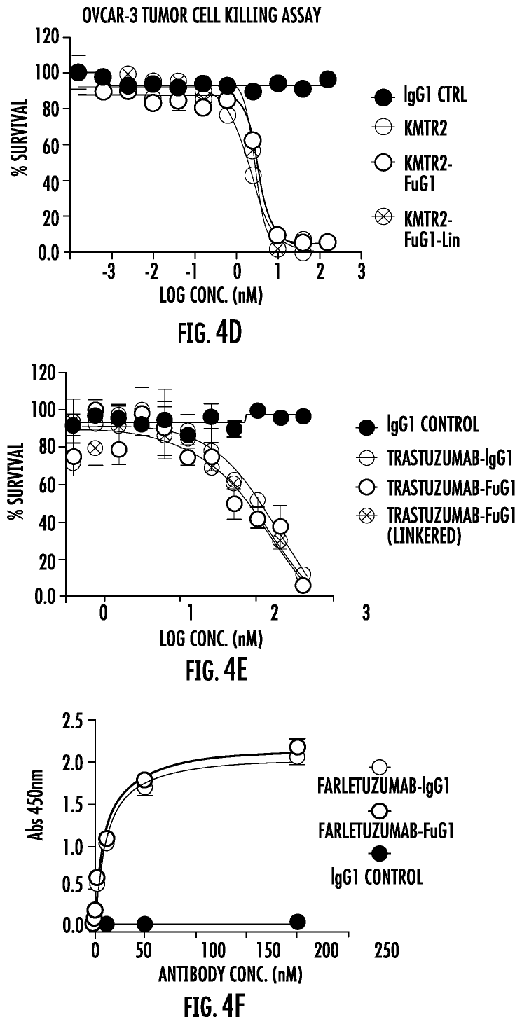

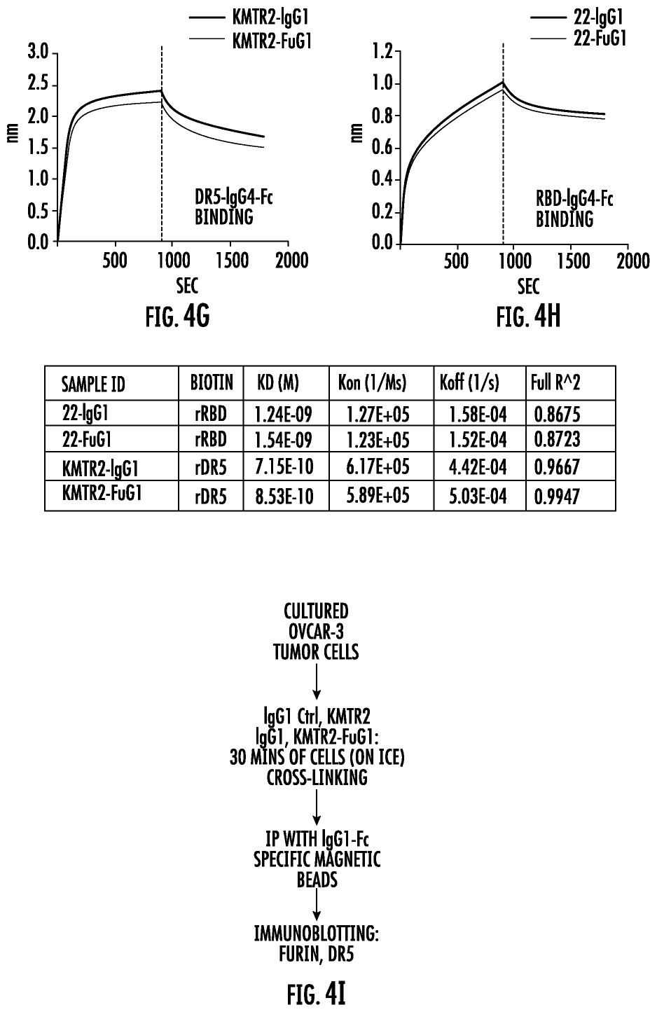

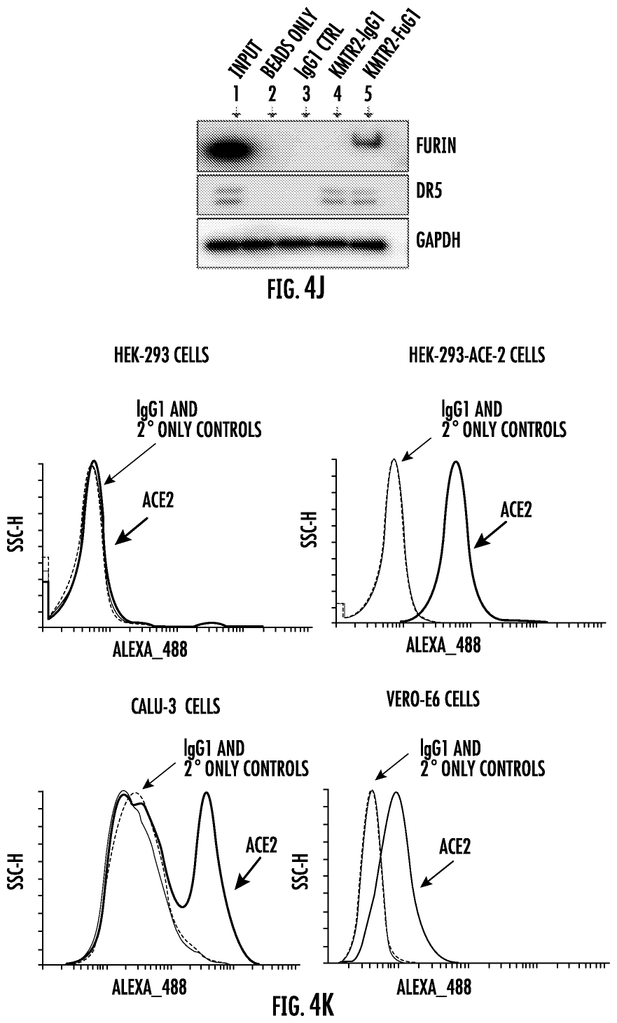

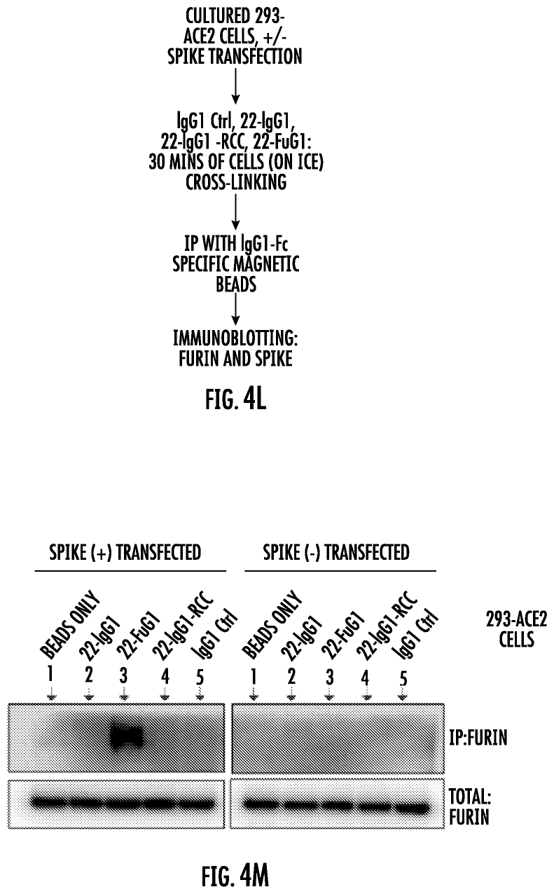

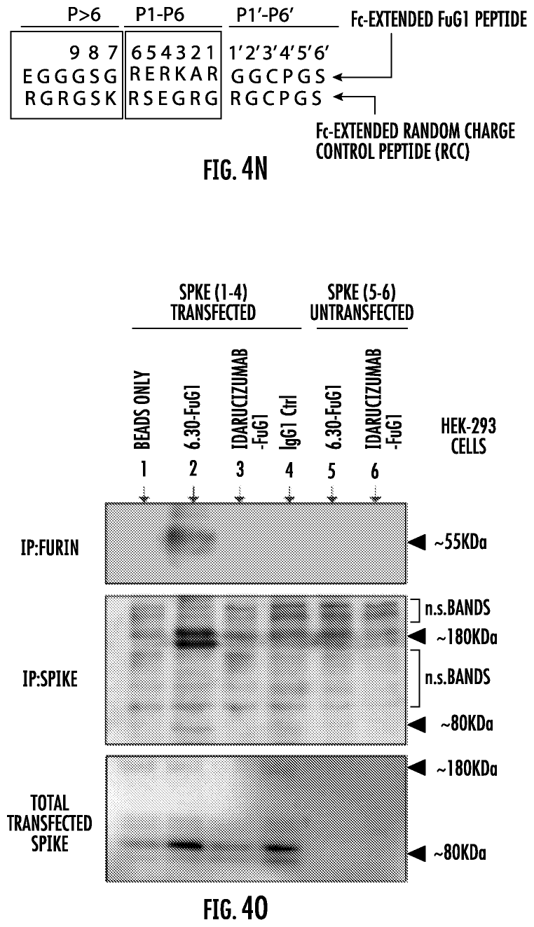

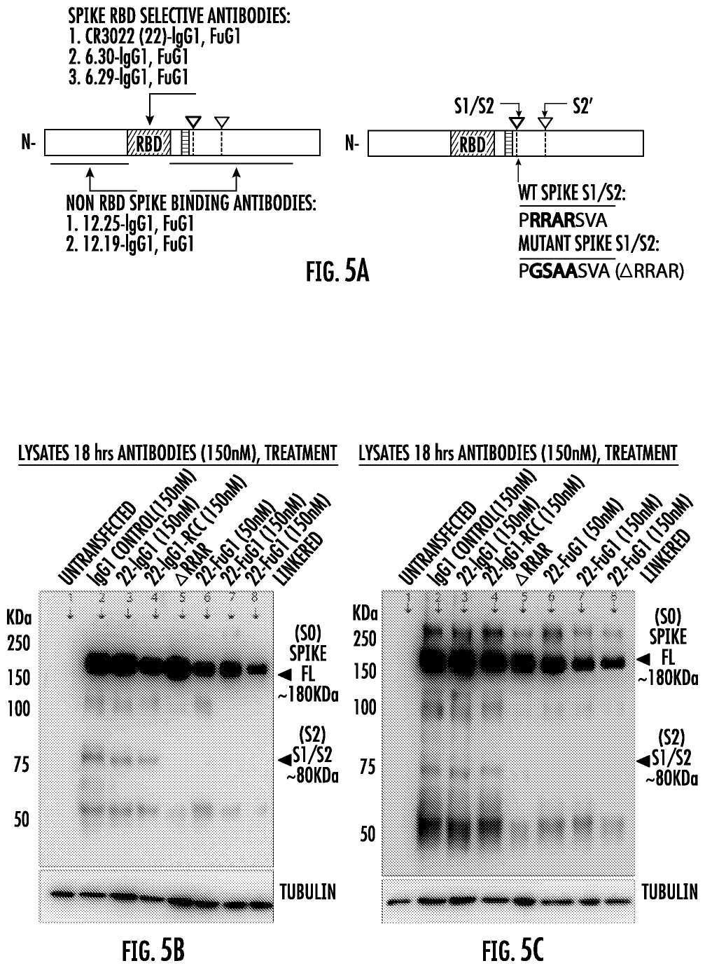

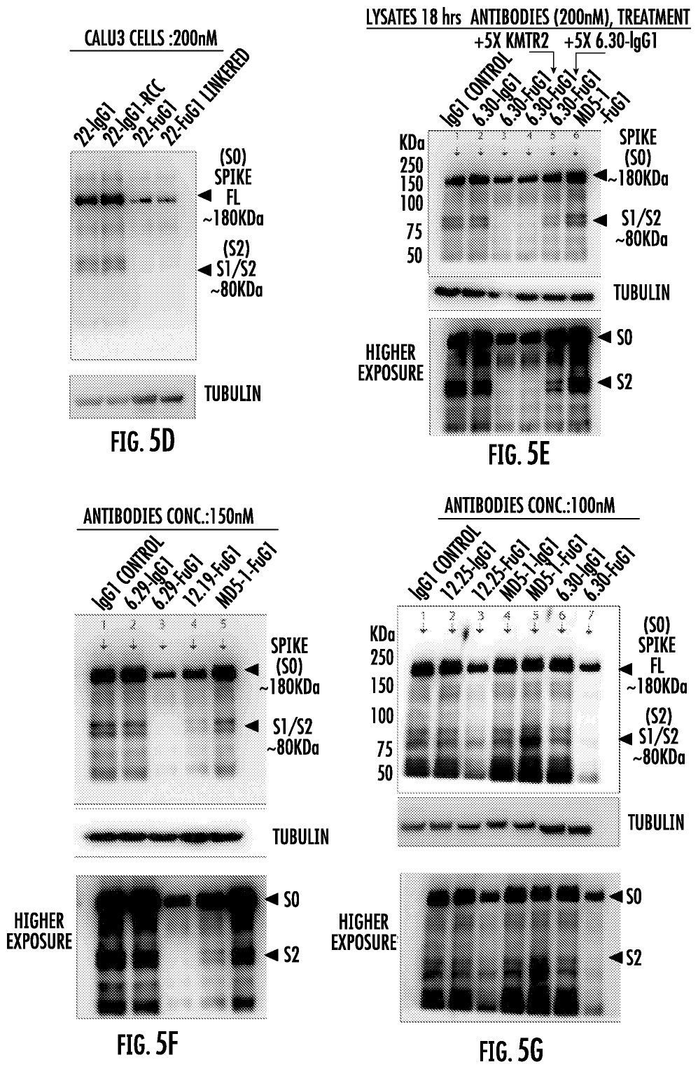

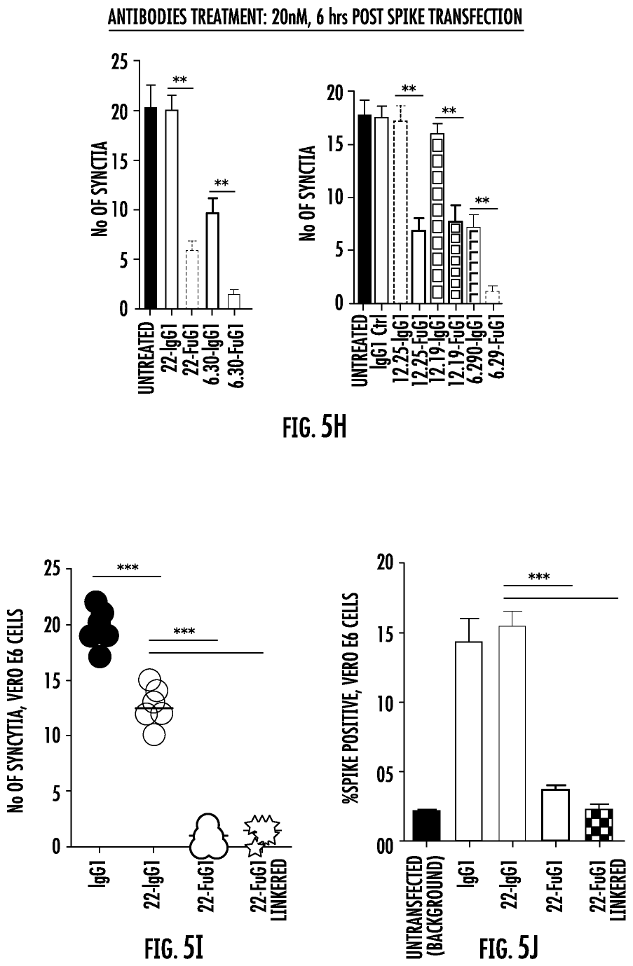

Recombinant antibody cloning. The sequence sources of various spike-targeting antibodies used in this study is provided. Additional antibodies such as farletuzumab, KMTR2, avelumab, idarucizumab clones has been published by our research group and are described in, for example, Shivange et al., 2018; Shivange et al., 2020; and Mondal et al., 2021a. Generation of linkered IgG1 has been described earlier by our group, see FIG. 1 in Shivange et al., 2018. Briefly, the DNA sequences were retrieved from the open sources (IMGT.org or publicly available patents or NCBI etc.) and synthesized as gene string with overlapping region to pCDNA3.1 vector using Invitrogen GeneArt gene synthesis services. Using overlapping pCDNA3.1 restriction site EcoR1 and HindIII primers, PCR amplification was carried out of gene VH and VL gene strings separately. After PCR amplification, DNA was gel purified and inserted into pCDNA 3.1 vectors (CMV promoter) by making use of In-Fusion HD Cloning Kits (Takara Bio). EcoR1 and HindIII digested vector was incubated with overlapping PCR fragments (of various different recombinant DNAs, see list of clones, FIG. 4 and FIG. 5A) with infusion enzyme (1:2, vector: insert ratio) at 55° C. for 30 minutes, followed by additional 30 minute incubation on ice after adding E. coli STELLAR™ cells (Clontech). Transformation and bacterial screening were carried out using standard cloning methods. Positive clones were sequenced confirmed in a 3-tier method. Confirmed bacterial colonies were Sanger sequencing upon PCR followed by re-sequencing of mini-prep DNA extracted from the positive colonies. Finally, maxiprep were re-sequenced prior to each transfection. Recombinant antibodies were also re-confirmed by ELISA and flow cytometry surface binding studies as described in Shivange et al., 2018. FuG1 antibodies were also engineered in similar way and the detailed sequence of FuG1 Fc-extended peptides is provided in the manuscript figures. Linkered monospecific and bispecific with knob and hole mutations were generated using flexible linkers as described in Shivange et al., 2018.

Recombinant RBD IgG4-Fc cloning. To clone spike RBD domain, amino acid 333-530 were order for gene string synthesis in continuation of G4S flexible linker and IgG4 CH2 and CH3 domain with EcoR1 and HindIII overlapping region. Following PCR amplification with overlapping primers, DNA was gel purified and inserted into pCDNA 3.1 vectors (CMV promoter) by making use of In-Fusion HD Cloning Kits (Takara Bio) as described in previous section. Recombinant IgG4-Fc FOLR1 and DR5 were also cloned in similar manner and have been described previously in Shivange et al., 2018.

Recombinant antibody and IgG4-Fc DR5, RBD, FOLR1 expression. Free style CHO—S cells (Invitrogen) were cultured and maintained according to supplier's recommendations (Life Technologies) biologics using free style CHO expression system (life technologies) and as previously described in, for example, Durocher & Butler, 2009 and Shivange et al., 2018. A ratio of 1:2 (light chain, VL: heavy chain, VH) DNA was transfected using 1 μg/ml polyethyleniamine (PEI). After transfection cells were kept at 37° C. for 24 hrs. After 24 hours, transfected cells were shifted to 32° C. to slow down the growth for 9 additional days. Cells were routinely feed (every 2nd day) with 1:1 ratio of Tryptone feed and CHO Feed B. After 10 days, supernatant from cultures was harvested and antibodies were purified using protein-A affinity columns. Various recombinant antibodies used in this study and recombinant target antigens were engineered, expressed, and purified in Singh Laboratory of Novel Biologics as described in Shivange et al., 2018. Recombinant antigens were similar expressed and transfected with PEI at 1 μg/ml concentration. All purified proteins were also confirmed for size using reducing SDS-PAGE, and standard ELISA as described in Shivange et al., 2018.

Antibody purification. Various transfected IgG1 antibodies, FuG1 antibodies and recombinant IgG4-Fc antigens (as indicated in text and figure legends) were affinity purified using HITRAP™ MABSELECT SURE™ (GE, 11003493) protein-A columns. Transfected cultures were harvested after 10 days and filtered through 0.2-micron PES membrane filters (Milipore Express Plus). Cleaning-in-place (CIP) was performed for each column using 0.2M NaOH wash (20 min). Following cleaning, columns were washed 3-times with Binding buffer (20 mM sodium phosphate, 0.15 M NaCl, pH 7.2). Filtered supernatant containing recombinant antibodies or antigens were passed through the columns at 4° C. Prior to elution in 0.1 M sodium citrate, pH 3.0-3.6, the columns were washed 3 times with binding buffer (pH 7.0). The pH of eluted antibodies was immediately neutralized using sodium acetate (3M, pH 9.0). After protein measurements at 280 nm, antibodies were dialyzed in PBS using Slide-A-Lyzer 3.5K (Thermo Scientific, 66330). Antibodies were run on gel filtration columns (next section) to analyze the percent monomers. Whenever necessary a second step size exclusion chromatography (SEC) was performed. Recombinants IgG4-Fc tagged extracellular domain antigens such as rFOLR1, rDR5 and RBD etc. were also similarly harvested and purified using protein-A columns.

Size exclusion chromatography. The percent monomer of purified antibodies was determined by size exclusion chromatography. 0.1 mg of purified antibody was injected into the AKTA protein purification system (GE Healthcare Life Sciences) and protein fractions were separated using a Superdex 200 10/300 column (GE Healthcare Life Sciences) with 50 mM Tris (pH 7.5) and 150 mM NaCl. The elution profile was exported as Excel file and chromatogram was developed. The protein sizes were determined by comparing the elution profile with the gel filtration standard (BioRad 151-1901; Hong et al., 2012). Any protein peak observed in void fraction was considered as antibody aggregate. The area under the curve was calculated for each peak and a relative percent monomer fraction was determined as described in Shivange et al., 2018.

Binding studies by ELISA. Binding specificity and affinity of various described IgG1's and FuG1 antibodies (including linkered FuG1, FuG1-Lin) s were determined by ELISA using the recombinant extracellular domain of corresponding receptor/target antigen. For coating 96-well ELISA plates (Olympus), the protein solutions (2 μg/ml) were prepared in coating buffer (100 mM Sodium Bicarbonate pH 9.2) and 100 μl was distributed in each well. The plates were then incubated overnight at 4° C. Next day, the unbound areas were blocked by cell culture media containing 10% FBS, 1% BSA and 0.5% sodium azide for 2 hours at room temperature. The serial dilutions of antibodies (2-fold dilution from 50 nM to 0.048 nM) were prepared in blocking solution and incubated in target protein coated plates for 1 hour at 37° C. After washing with PBS solution containing 0.1% Tween20, the plates were incubated for 1 hour with horseradish peroxidase-(HRP) conjugated anti-human IgG1 (Thermo Scientific, A10648). Detection was performed using a two-component peroxidase substrate kit (BD Biosciences) and the reaction was stopped with the addition of 2N Sulfuric acid. Absorbance at 450 nm was immediately recorded using a Synergy Spectrophotometer (BioTech), and background absorbance from negative control samples was subtracted. The antibody affinities (Kd) were calculated by non-linear regression analysis using GraphPad Prism software.

Flow cytometry. The cell surface expression of ACE2, Spike, DR5, FOLR1, etc. was analyzed by flow cytometry in various experiments (see Figures and corresponding Brief Descriptions thereof) after treatment with various control and FuG1 antibodies. After various treatments, cells were trypsinized and suspended in FACS buffer (PBS containing 2% FBS). The single cell suspension was then incubated with primary antibodies for 1 hour at 4° C. with gentle mixing. Following wash with FACS buffer, the cells were then incubated with fluorescently labeled anti-Rabbit antibody for 1 hour. Cells were washed and flow cytometry was performed using FACSCalibur. The data was analyzed by FCS Express (De Novo Software) and FlowJo.

Binding studies by BioLayer Interferometry (BLI). Binding measurements were performed using Bio-Layer Interferometry on FortéBio Red Octet 96 instrument (Pall) as described in Shivange et al., 2018. Biotin-Streptavidin based sensors were employed for the studies. Recombinant Fc linked DR5 variants were biotinylated using EZ-Link Sulfo-NHS—SS-Biotin (Thermo Scientific 21331) following the manufacturer's instructions. Unreacted Sulfo-NHS—SS-Biotin reaction was quenched by 50 mM Tris-Cl pH 7.4 and removed via dialyzing against PBS. For binding analysis biotinylated antigen (1 μg/mL) were immobilized on streptavidin (SA) biosensors (Pall) for 300 sec to ensure saturation. Associate and dissociation reactions were set in 96-well microplates filled with 200 μL of unbiotinylated DR5 agonist for 900 seconds. All interactions were conducted at 37° C. in PBS buffer containing 2 mg/ml BSA. These binding observations were also confirmed by biotinylating the agonist antibodies and probed against unbiotinylated DR5-Fc variants. Kinetic parameters (K_{ON} and K_{OFF}) and affinities (KD) were analyzed using Octet data analysis software, version 9.0 (Pall).

Furin protease activity assay. Different concentrations of farletuzumab-FuGi, farletuzumab-cFuGi (25-250 ng) or furin assay buffer (negative control) were pre-incubated with 25 ng of furin (50 μl, 0.5 ng/μl in furin assay buffer) in a total reaction volume of 100 μl for 10 minutes at room temperature. The reaction was started by adding 40 μl furin protease substrate (final concentration of the furin protease substrate in a 100 μl reaction was 2 μM). The fluorescence intensity of the reaction mixture was monitored at excitation wavelength of 380 nm and detection of emission at wavelength 460 nm using 96 well microplate reader (BioTek Instruments Inc, USA). For inhibitor control, 10 μl of chloromethylketone (0.5 μM) was added to 25 ng of furin and for positive control (only furin), no antibody and inhibitor were added. The protease activity of positive control well was considering as 100% activity and the decrease in furin activity was compared to that. For kinetic study, fluorescence was measured immediately after adding the protease substrate and data was recorded every 1 minute for 15 min at Ex/Em=380 nm/460 nm.

General western blotting. Cells were cultured overnight in tissue culture-treated 6-well plates prior to treatment. After antibody treatment for 48 hours (or indicated time), cells were rinsed with PBS and then lysed with RIPA buffer supplemented with protease inhibitor cocktail (Thermo Scientific). Spinning at 14000 rpm for 30 minutes cleared Lysates and protein was quantified by Pierce BCA protein assay kit. Western blotting was performed using the Bio-Rad SDS-PAGE Gel system. Briefly, 30 μg of protein was resolved on 10% Bis-Tris gels and then transferred onto PVDF membrane. Membranes were blocked for one hour at room temperature in TBS+0.1% Tween (TBST) with 5% non-fat dry milk. Membranes were probed overnight at 4° C. with primary antibodies. Membranes were washed three times in TBST and then incubated with anti-rabbit or anti-mouse secondary antibodies (1/10,000 dilution, coupled to peroxidase) for 1 hour at room temperature. Membranes were then washed three times with TBST and Immunocomplexes were detected with SuperSignal West Pico Chemiluminescent Substrate (Thermo Fisher Scientific). Images were taken using a Bio-Rad Gel Doc Imager system.

Syncytia formation and inhibition studies in presence of FuG1 antibodies. 293T cells expressing stable ACE2 (293-ACE2) are described in Crawford et al., 2020 and were kindly provided Dr. Jesse D. Bloom. 293-ACE2 were transfected with 1 μg (unless mentioned) of Spike-EGFP plasmid using Mirus reagent as per manufacturer's instructions. Cells were monitored for the formation of syncytia after 20 hours of incubation. In case of syncytia inhibition experiment, various control and FuG1 antibodies were added with indicated concentrations (see Figures and corresponding Brief Descriptions thereof for specific concentrations), 4 hours after transfection. For some experiments, such as those described for delayed farletuzumab-FuG1 treatments, various control and FuG1 antibodies were added either 12 hours or 14 hours after spike transfection to allow effective spike cellular expression (as indicated in Figures and corresponding Brief Descriptions thereof).

Cell extract and pseudoviral spike immunoblotting. For cellular spike processing analysis, indicated HEK-293 or ACE stable 293 cells, VERO-E6 or Calu3 cells (70-80% confluent) were transfected with 1 μg (unless mentioned) of WT or Spike ΔRRAR (furin mutant spike) using reagents from Mirus Bio LLC (Madison, Wisconsin. United States of America). Various indicated control and experimental FuG1 antibodies were added on cells (at indicated concentrations) 4 hours post DNA transfection. 24 hours (or else indicated) lysates were prepared and separated using 8-10% SDS-PAGE followed by immunoblotting either using full spike antibodies (1A9 GTX 632604, AB1N1030641). For immunoblotting analysis of pseudoviral spike processing, 1 ml respective pseudotypes (rVSV-Spike*ΔG, or rVSV-ΔRRAR-Spike*ΔG etc) either generated in presence of IgG1 control or spike targeting IgG1 antibodies (22-IgG1, 6.30-IgG1, 12.25-IgG1, 12.19-IgG1, 6.29-IgG1) or spike targeting FuG1 antibodies (22-FuG1, 6.30-FuG1, 12.25-FuG1, 12.19-FuG1, 6.29-FuG1 etc.) as indicated in the corresponding Figures were added on to sucrose gradient (20% w/v) followed by high-speed centrifugation (25,000 g, 4 hours) to break open the viral proteins. The pellet was added with 2×SDS loading dye and incubated at 95° C. in PCR machine for 10 minutes. Samples were later subjected to SDS-PAGE immunoblotting using Spike, VSV-G, VSV-M or other indicated antibodies. VSV-matrix protein (VSV-M) served the pseudoviral loading control

Production of replication defective SARS-CoV-2 Spike (rVSV-Spike*ΔG) pseudovirions. rVSV-Spike*ΔG were generated as described in Whitt, 2010 and Hoffmann et al., 2020a. Replication-restricted VSV-G pseudotyped ΔG-luciferase (VSV-G*ΔG-luciferase) rVSVw/pCAGGS-G-Kan (EH1025-PM) and BHK1 cells (EH1011) were purchased from Kerafast. In addition, SARS-CoV2 S protein pseudotyped Luc reporter virions were also purchased for ready to do experiments from BPS Biosciences pseudovirus (Cat #79942) and (Virongy (CoV2Luc-02). Titer of these pseudovirions were determined as per manufacture recommendations. rVSV-Spike*ΔG pseudovirions were generated next to VSV-G pseudovirions in culture conditions as VSV-G induced syncytia in BHK-21 cells helped timely monitoring of spike pseudovirions recovery as described in Whitt, 2010 and Hoffmann et al., 2020a. We generated rVSV-Spike*ΔG with both 293T and BHK1 cells. WT and furin mutant: ΔRRAR spike (ordered as gene blocks, Life Technologies) were cloned and sequence confirmed in pCAGGS-G-amp vector, kindly provided by Dr. Michael Whitt. Briefly, 10 cm plates with 80% confluency of 293T or BHK1 cells (37° C. with 5% CO₂ overnight) were transfected with pCAGGS-spike, pCAGGS-ΔRRAR-spike (or pCAGGS-VSV-G) plasmids for the respective WT spike, furin mutant spike (or VSV-G) proteins expression, using mirus transfection (MirusBio) reagent. At 16 hours post transfection, the transfected cells were transduced with replication defective VSV-G*ΔG-luciferase (Kerafast), which encodes for luciferase. Cells were further incubated for 24 hours, until the majority of cells in a side-by-side VSV-G transfected manner induced CPE. After that supernatants were collected and clarified by centrifugation at 1000 rpm for 5 min, passed through 0.45 μm PES filter, and concentrated using 10% (w/v) PEG 6000 and 5M NaCl. PEGylation solution was mix and incubated at 4° C. overnight on gentle rocker. The precipitated pseudovirion particles were centrifuged at 4000 rpm for 30 min and resuspended in 1 ml of culture media. Pseudovirion particles were either used immediately or aliquoted prior to storage in −80° C. to avoid multiple freeze-thaws. To transduce cells with pseudovirions, ˜2×10⁵ 293-ACE2 cells were seeded in a 96 well plate 20-24 hours prior to inoculation. A single aliquot of pseudovirions was used for infection. After overnight incubation, cell lysates were generated and transferred to white wall reader plated. 5 μl D-luciferin (300 μg/ml) reagent per well was added and incubated at room temperature for indicated time. The transduction efficiency was measured by quantification of the luciferase activity using a Synergy HT 96 well microplate reader (BioTek Instruments Inc, USA). All experiments were done in triplicates and repeated at least twice or more.

Pseudovirions generation in presence of FuG1 antibodies. As VSV-G induces highly effective syncytia in BHK-21, replication defective pseudovirions SARS-CoV-2 pseudovirions (rVSV-Spike*ΔG) were generated next to VSV-G*ΔG-pseudovirions in culture conditions as described in Whitt, 2010 and Hoffmann et al., 2020a. Briefly, 10 cm plates with 80% confluency of 293T or BHK1 cells (37° C. with 5% CO₂ overnight) were transfected with pCAGGS-spike, pCAGGS-ΔRRAR-spike (or pCAGGS-VSV-G) expression plasmids for the respective WT spike, furin mutant spike (or VSV-G) proteins using mirus transfection reagent. 6 hours later, various spike targeting (RBD, non-RBD-binding) antibodies were added to the cultures at indicated concentration. At 24 hours post transfection, the transfected cells were re-treated with corresponding antibodies (FuG1 or controls) along with the inoculation of luciferase encoding replication defective VSV-G*ΔG-luciferase (Kerafast). Cells were further incubated for additional 24 hours, before the rVSV-Spike*ΔG, rVSV-ΔRRAR-Spike*ΔG, or (rVSV-G*ΔG) containing supernatants were harvested, followed by centrifugation and filtration to eliminate cellular debris. Pseudovirion particles were either used immediately or aliquoted prior to storage in −80° C. to avoid multiple freeze-thaws.

Spike pseudoviral transduction and host cell entry assays. Briefly, 293-ACE2 cells were seeded at a density of 60-70% confluency into a 96-well cell culture plate in the DMEM media. Cells were incubated at 37° C. with 5% CO₂ overnight. Next day, media was aspirated from the cells and approximately 100 ml/well of the respective pseudotypes (rVSV-Spike*ΔG) either generated in presence of IgG1 control or spike targeting IgG1 antibodies (22-IgG1, 6.30-IgG1, 12.25-IgG1, 12.19-IgG1, 6.29-IgG1) or spike targeting FuG1 antibodies (22-FuG1, 6.30-FuG1, 12.25-FuG1, 12.19-FuG1, 6.29-FuG1 etc.) as indicated in figures were added in triplicate. After 24 hours post pseudoviral transduction, media was aspirated from the cells, followed by cell lysis. The lysates were transferred to corresponding 96 well white wall, clear bottom plates. 5 μl D-luciferin (300 μg/ml) reagent was added per well and incubated at room temperature for luciferase activity signal. The transduction efficiency was measured by quantification of the luciferase activity using a Synergy HT 96 well microplate reader (BioTek Instruments Inc, USA). All experiments were done in triplicates and repeated at least twice or more. Data was plotted using GraphPad Prism software.

Crude endosomal (CE) lysate fractionation. A discontinuous gradient of 8%, 30% and 42% was generated to collect the pellet at 8% to 30% interface as described earlier (29). Briefly, spike transfected cells (20 hours) were washed with PBS followed by whole cell lysates generation using lysis buffer (300 mM sucrose, 10 mM Tris pH 8.0. 5 mM CaCl₂), 3 mM imidazole, pH 7.4, 1 mM EDTA, 3 mM Mg acetate, 1 mM DTT, 0.1% Triton, ddH₂O, protease and phosphatase inhibitor). After stepwise centrifuge steps (500 g, 2000 g, 3000 g, 4° C.), post nuclear supernatants (PNS) containing various the cell organelles in suspension were recovered. Additional centrifugation could be carried out at this step to enrich nuclei. The PNS were subsequently separated by discontinuous gradient ultracentrifugation. After recovery, the sucrose concentrations of the PNS were adjusted to 40% by adding 62% sucrose, usually with 1:1.2 V/V (percent volume per volume). PNS with 40% sucrose was loaded on the bottom of an ultracentrifuge tube. This was followed by sequential overlay with 30% sucrose and 8% sucrose. Interphases of three gradients were marked the waterproofed pen. This was followed by ultracentrifugation at 210,000×g at 4° C. for 2 hours. After centrifugation, we detected clear milky band of membrane particles at the interface of 8% and 30% sucrose called crude endosome (CE) fraction. The interface at 30% and 42% was diffused and significantly dense. As described in de Araujo et al., 2015, floating late endosomes, smaller size lysosomes, early endosomes and carrier recycling vesicles are concentrated at the interface between 8% and 30% sucrose (CE), while the heavy membranes like Golgi and ER and enlarged lysosomes membranes are enriched at second interface. After recovery, proteins were measured and approximately 10 mg CE samples were loaded along with total lysates (15 mg) for immunoblotting.

ER, Trans-Golgi-Network and other membrane enrichment using commercial kits. ER and Golgi fractions from spike transfected cells were enriched using commercial Minute enrichment kits, cat #ER-036 and cat #ER-037 respectively from Invent Biotechnologies. Briefly, 293-ACE cells were grown in 150 cm² dishes and were divided into various groups. At ˜70-80 confluency cells were transfected with spike. 24 hours later supernatant was removed, and cell were collected by scraping followed by low-speed centrifugation (500-600×g for 5 minutes) and cold PBS wash. Cell pellet was resuspended in 550 μl of buffer A (manufacturer provided in the kit) and vortexed followed by transfer of cell suspension to manufacturer supplied filter cartridge and inverted few times. After that multiple centrifugation steps were carried out as per manufacturer recommendations. After separating mitochondria, ER, lysosomes and plasma membranes, 400 μl buffer B (manufacturer provided in the kit) was added to the recovered supernatant and mixed by vortexing briefly. The tube was incubated on ice for 15 minutes followed by additional centrifuge at 8,000×g for 5 minutes and supernatant with secretory vesicles of trans-Golgi was transferred to a fresh tube which was later concentrated. To enrich for cis Golgi fraction additional recommended centrifugations were carried out and pellet was suspended in 1×PBS. Lysates were mixed with protease inhibitor, SDS loading dye was added. Samples were boiled at 95 degree for 10 minutes before loading on the gel for immunoblotting. Specific enrichment of Golgi fractions was confirmed by immunoblotting against GM130 (Cell signaling: 12480S), golgin-97 (Cell signaling: 13192S). After Golgi fractionations, the cat #ER-037 kit also enriches remaining membrane fraction which includes lysosomes, ER, late and recycling endosomes membranes. Similar procedure was carried out using ER enrichment kit, Cat #ER-036 as per manufacturer recommendations. Selective enrichment of ER fractions was confirmed by immunoblotting against ER resident chaperone GRP78/BIP (Cell signaling: 3177S). For ER-TGN transport studies, we made use of Brefeldin A (BFA). BFA was purchased from Biolegend (420601S) and a working 5 mg/ml stock solution in ethanol was prepared. For ER to Golgi transport inhibition studies of spike, cells were treated with BFA at the time of spike transfection. 24 hour lysates were analyzed for immunoblotting using spike antibodies along with total lysates.

Quantitation and statistical analysis. Data unless indicated otherwise are presented as mean±SEM. In general, when technical replicates were shown for in vitro experiments, student t-test was used for statistical analysis and the same experiment was at least repeated once with similar trend observed. When data from multiple experiments was merged into one figure, statistical significance was determined by either Wilcoxon Mann-Whitney test or unpaired T-test with Welch's correction using Graph Pad Prism 5.0 software. For all the statistical experiments p values, p<0.05 (*), p<0.01 (**) and p<0.001 (***) were considered statistically different or specific p values indicated otherwise or “ns” indicates non-significant.

### Example 1

### The FuG1 Strategy

It was hypothesized that a strategy capable of competitively inhibiting cellular furin and selectively targeting SARS-CoV-2 in the infected viral-producing cells plays a role in therapeutic specificity. Thus, a spike targeting IgG1-Fc-based design was engineered that would selectively compete with early, late, recycling endosomal, and cell surface enriched furin protease function at the site of potential spike trafficking, processing, and incorporation into the virus. The latter would significantly interfere with S1/S2 cleavage of the full-length spike (S0) to generate viral fusion ready S2 (S0: ˜180 kDa to S2: ˜80 kDa) fragment (see Cai et al., 2020; Lan et al., 2020). As a result, regulated spike viral incorporation and function in virus-producing cells would be significantly impeded to negatively impact the subsequent host-cell entry and SARS-CoV-2 chain of infection function. The simultaneous spike-targeting and competitively furin inhibiting schematic illustration of the FuG1 strategy is shown in FIGS. 1C-1E. To test the latter, we used SARS-CoV-2 RBD and non-RBD region binding antibodies (along with the design of various control antibodies) engineered to harbor highly competitive and furin engaging peptides in the Fc-extended flexibly linked region beyond the CH3 domain (FIG. 1D, pink when shown in color). This targeting approach was named “FuG1”, an anti-spike IgG1 that competitively engages furin.

Selection, design, and flexibility of optimal furin inhibiting FuG1 peptide. To design optimally competitive and furin engaging Fc-extended peptide in FuG1, the previously described PiTou tool composed of a hidden Markov model (see Tian et al., 2012) was used. Although the PiTou score ranges from 0.01-0.99 (see Tian et al., 2012), the corresponding scores in the higher range (>0.7-0.99) represent decidedly accurate furin cleavage prediction with the highest efficiency. Specifically, we enriched the P1′—P6′ region of the 18 amino acid Fc-extended peptide in FuG1 with nonpolar hydrophobic residues (FIG. 1F), including a side-chained cysteine (GGCSPG), as enrichment of hydrophobic residues (over hydrophilic) near P3′—P6′ region has been described to interfere with furin target cleavage efficiency (16). Besides, acidic anchor glutamate (E) was inserted at P5/P6 position (right next to the RKxR recognition sequence), which has been described to significantly inhibit furin catalysis without affecting its binding (see Kacprzak et al., 2004). As previous studies by Tian et al., 2012 and Omotuyi, 2015 have defined minimal loss of furin binding against computationally defined target sequences having predicted PiTou score close to cleavage threshold (0.5), an 18 amino acid FuG1 peptide with a cleavage score of 0.504 was chosen to competitively inhibit furin with multiple other control peptides (FIGS. 1F and 1G). Next, based on a range of PiTou scores, various Fc-extended peptides were inserted into recombinant death receptor-5, rDR5-IgG4-Fc (see Shivange et al., 2018) by replacing the region near its natural positively charged enriched (RKCR; SEQ ID NO: 40) motif (see Shivange et al., 2021) in the 3rd cysteine-rich domain (FIG. 1H, see the EEDSPE (SEQ ID NO: 62), MCRKCR (SEQ ID NO: 63), and TGCPRG (SEQ ID NO: 64) sequences). If rDR5-IgG4-Fc were to be cleaved by recombinant furin, two ˜30 kDa and ˜12-15 kDa fragments would be generated (FIG. 1H).

Next, we replaced wild-type rDR5-IgG4-Fc and various recombinant DR5-IgG4 Fc with RKCR region sequences with PiTou generated peptides (FIG. 1I). These constructs were expressed using CHO cells in the presence of furin inhibitor decanoyl-RVKR-CMK. The purified recombinant DR5 proteins were tested for cleavage according to the predicted score (FIGS. 1I and 1J). The selected lead FuG1 peptide (PiTou score of 0.504), when inserted in rDR5-IgG4-Fc, was not cleaved by recombinant furin in vitro (FIGS. 1I and 1J, ane 4). Two alternate and furin optimal Fc-extended peptides engineered with the sequence of spike S1/S2 residues (SVASQS) in P1′—P6′ region, when inserted in rDR5-IgG4-Fc, gave a high cleavage prediction score of 0.753 and 0.808, respectively (FIG. 1I). When tested in similar experimental conditions (within rDR5-IgG4-Fc) along with lead competitive peptides, both of these peptides resulted in expected ˜30 kDa and 12 kDa fragments when incubated with recombinant furin (FIG. 1J, lanes 1, 2 vs. 4). Upon further optimization of in vitro cleavage conditions with 20 mM KCl (pH 6.5 reaction buffer) and increased furin concentration (see Izidoro et al., 2009), we observed efficient cleavage of RRAR-rDR5-IgG4-Fc (cFuG1) but not of RKAR-rDR5-IgG4-Fc (FuG1; see FIG. 1K, lanes 1 and 2 vs. lanes 3 and 4). We chose the cleavable sequence with a 0.808 score as a control (cFuG1) for furin binding studies (FIG. 1I).

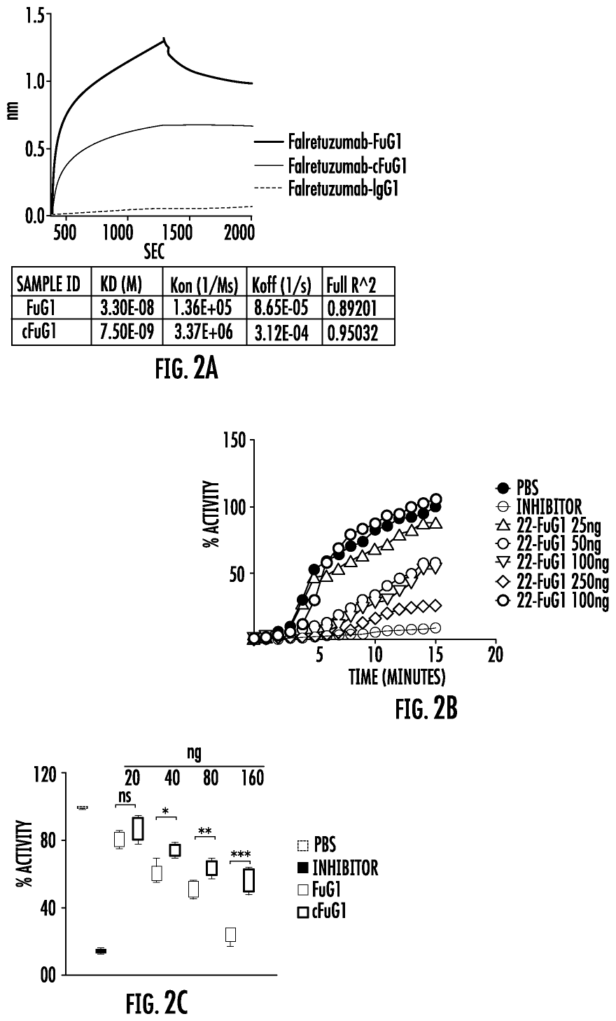

To confirm furin binding and competing function, farletuzumab-FuG1 and farletuzumab-cFuG1 were expressed in the presence of furin inhibitors. Purified antibodies were analyzed for their Fc-extended peptide binding to biotinylated furin using BLI. Although furin binding affinity (K_{D}) and association rate (K_{ON}) of FuG1 were lower (compared to cFuG1), the furin dissociation rate (K_{OFF}) of FuG1 was also almost 10-fold reduced (FIG. 2A). The latter indicated a potential higher competitive furin occupancy time of FuG1 peptide with the increased antibody concentration (Hoare et al., 2020). In support, when tested using an in vitro protease assay that relies on the generation of the fluorogenic substrate upon cleavage by recombinant furin (Jaimes et al., 2019), we observed significant inhibition by Farletuzumab-FuG1 antibody, particularly at higher doses when compared with the control farletuzumab-cFuG1 containing peptide (FIGS. 2B and 2C).

### Example 2

### Trans-Golgi-Network (TGN) Independent Processing of Spike

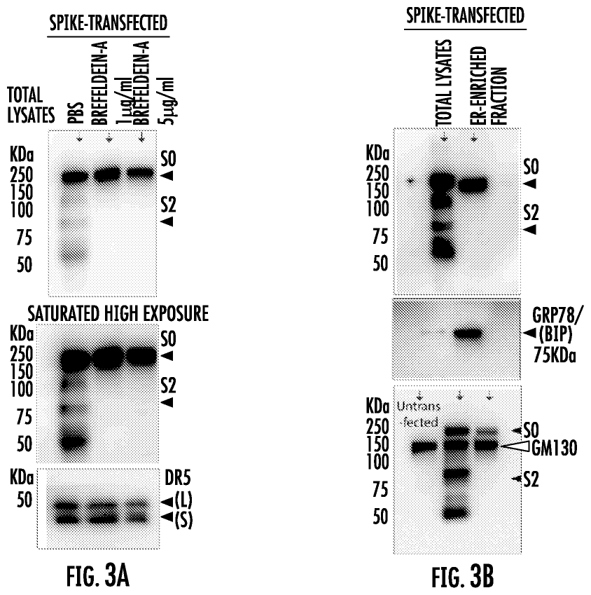

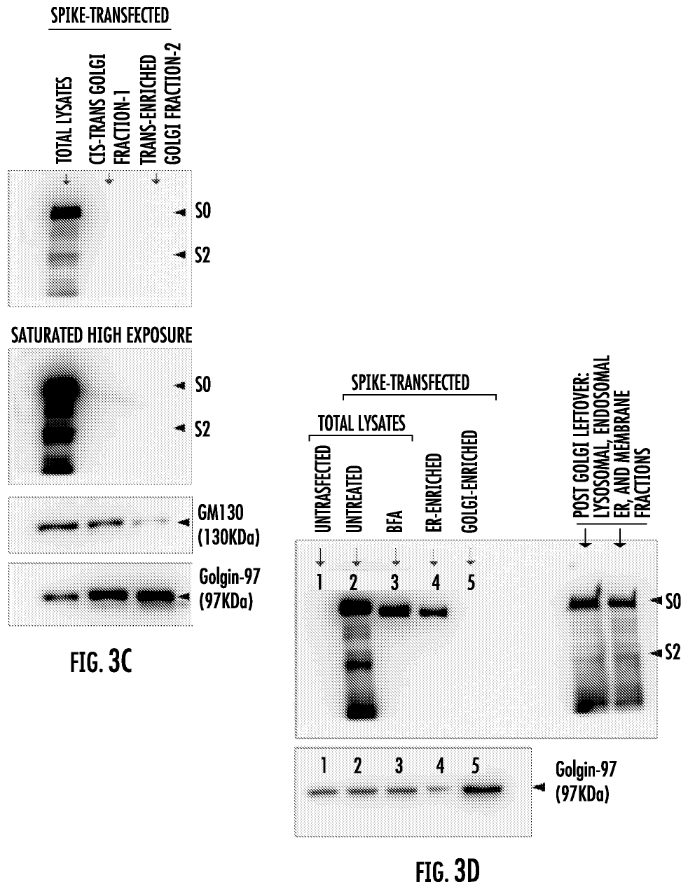

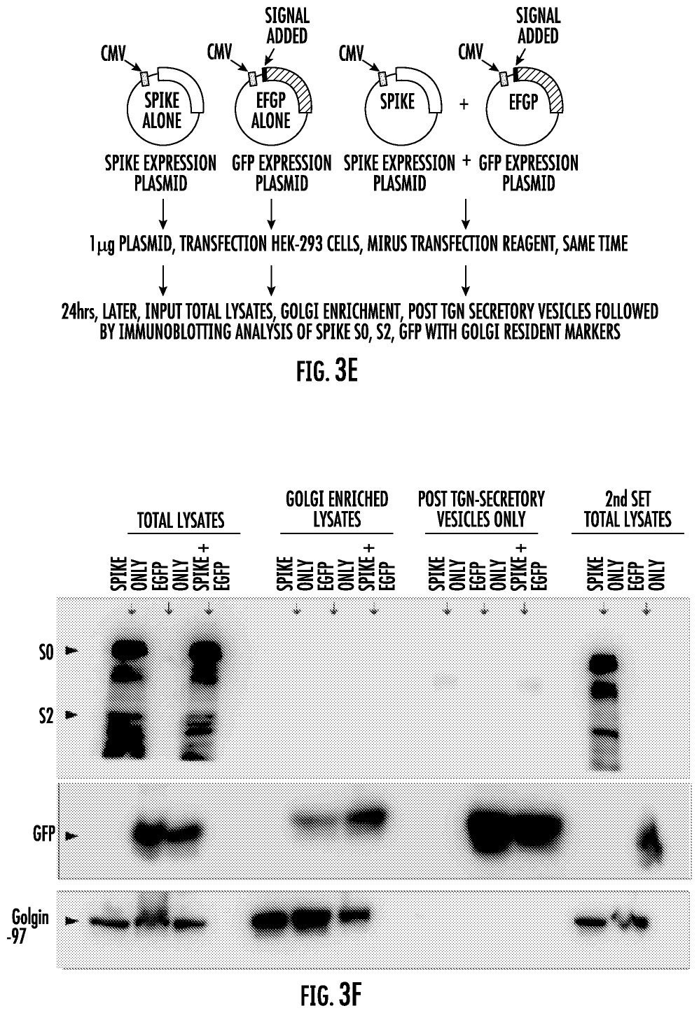

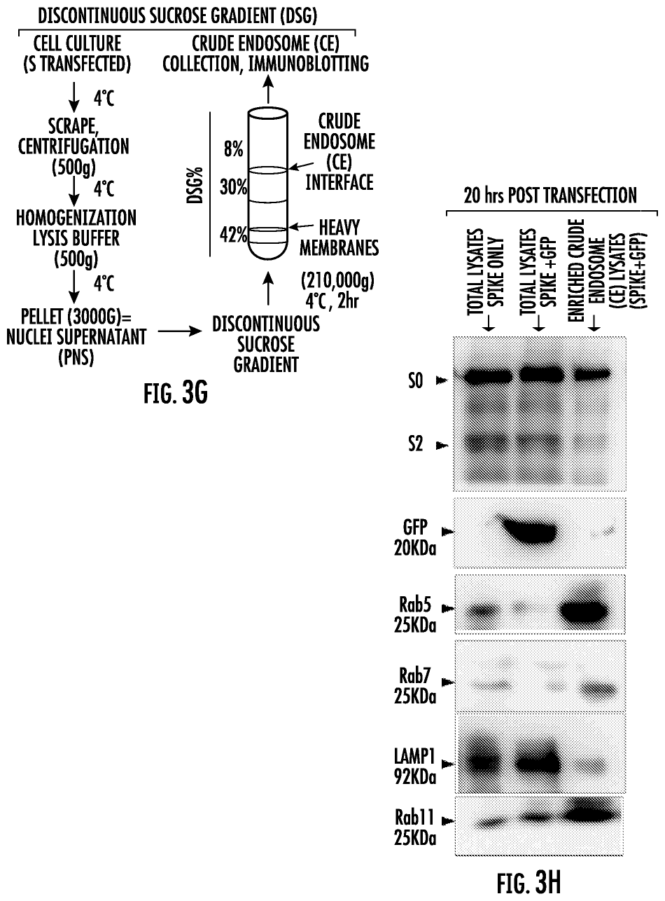

It is believed that trans-Golgi-network (TGN) enriched furin cleaves spike at S1/S2 sites. However, no study has conclusively proved the selective role of the TGN secretory pathway in spike processing, despite the well-established additional regulatory furin protease functions in early, late, recycling endosomes and plasma membrane in mammalian cells (see Klimpel et al., 1992; Thomas, 2002). Specifically, adaptor proteins and regulatory modifications of protein tethers regulate TGN independent furin activity in sorted late or recycling endosomes and at plasma membrane independent of TGN function (Tsuneoka et al., 1993; Thomas, 2002; Koo et al., 2006). Thus, various furin processing mechanisms exist to generate functional effector proteins (Koo et al., 2006). In light of recently published studies of TGN independent lysosomal deacidification route of SARS-CoV-2 (Ghosh et al., 2020), we sought to investigate if spike S1/S2 cleavage event happens in TGN or post-TGN endosomal-cell surface recycling route. To this end, spike transfected HEK-293-ACE2+ and HEK-293-ACE2⁻ cells were treated with endoplasmic reticulin (ER) to TGN trafficking inhibitor Brefeldin-A (Bfa). Bfa completely inhibited spike S1/S2 cleavage (FIG. 3A), ruling out ER cleavage of the spike, which was reconfirmed via enriching ER-resident chaperone GRP78/BIP containing fractions (FIG. 3B; Park et al., 2017).

Next, we enriched both cis and trans-Golgi compartments to analyze S1/S2 cleavage using the cell fractionation kit. Surprisingly, despite repeated testing, full-length spike (S0) expression was not detected in TGN compartments even though a significant accumulation of cis and trans-Golgi compartments resident markers GM130 and golgin-97, respectively, was consistently evident (FIGS. 3C and 3D). To further reconfirm these studies, we co-transfected spike expression plasmid and GFP expression plasmid (GFP with N-terminal membrane signal peptide) together and separately followed by Golgi compartments and post TGN secretory vesicle enrichment (FIG. 3E). When these fractions were immunoblotted for spike and GFP, GFP protein followed the traditional route of shuttling from TGN to secretory vesicle independent of being expressed alone or together with the spike protein (FIG. 3F). On the contrary, despite enrichment of GFP and resident golgin-97 protein, spike signal was completely absent in TGN and post TGN secretory vesicles (FIG. 3F). These results strongly support TGN-independent sorting of spike (see e.g., Ghosh et al., 2020) and underpin lysosomal deacidification followed by late, early and recycled endosomal exit route to plasma membrane. Besides, spike cleavage was evident when after TGN fractionation, leftover intracellular membrane fractions (other than Golgi and plasma membrane) that include lysosomes, endosomes, mitochondria, and ER were enriched (FIG. 3D, last two lanes). To further demonstrate lysosomal/endosomal system-mediated spike cleavage, we enriched crude endosomal (CE) fractions via discontinuous sucrose gradient as described in de Araujo et al., 2015 (FIG. 3G; see also Materials and Methods Employed in the EXAMPLES above). Evidently, early (Rab5), recycling (Rab11), and late endosomal (Rab7) resident proteins enriched in CE fraction, with visible spike cleavage (FIG. 3H).

### Example 3

### FuG1 Peptide does not Interfere with the Target Binding and Cellular Function of Antibodies

Target-binding mediated endocytosis of IgG1 results in the shuttling of various surface receptor-bound therapeutic antibodies to recycling endosomes (Oganesyan et al., 2014; Blumberg et al., 2019) or in the lysosomes (Ritchie et al., 2013). Thus, we hypothesized significant blockade of S1/S2 cleavage by spike targeting FuG1 strategy mediated competitive inhibition of furin in the lysosomal/endosomal compartments and at cell membrane during spike trafficking to surface. To functionally test and strengthen biosynthetic secretory TGN pathway independent cleavage of the spike, along with SARS-CoV and SARS-CoV-2 RBD binding antibody CR3022-IgG1 (22-IgG1 hereafter), multiple other spike RBD and non-RBD targeting antibodies were converted into FuG1 strategy (FIGS. 4A-4C and 5A). In reducing conditions, the heavy chain (VH) of various engineered FuG1 antibodies had ˜3 kDa higher molecular weight while the light chain (VL) remains of the same size (FIGS. 4B and 4C).

Next, we confirmed that the addition of the Fc-extended furin linker peptide did not affect the function and binding of various FuG1 antibodies. For functional assays, we tested death-receptor-5 (DR5) targeting KMTR2-IgG1 vs. KMTR2-FuG1 and human epidermal growth factor receptor-2 (HER2) targeting Trastuzumab-IgG1 vs. Trastuzumab-FuG1. FuG1 conversion did not affect the function of anti-DR5 and anti-HER2 targeting clinical antibodies (FIGS. 4D and 4E). Similar results were evident in binding assays with anti-DR5 and anti-FOLR1 targeting FuG1 antibodies (FIGS. 4F and 4G) and spike RBD binding antibody 22-IgG1 and 22-FuG1 antibodies (FIG. 4H).

We next texted Spike-FuG1-Furin interactions with immunoprecipitation studies. When tested using ovarian cancer cells (OVCAR-3), death-receptor-5 (DR5) targeting KMTR2-IgG1 and KMTR2-FuG1 antibodies both pulled down DR5; however, only KMTR2-FuG1 interacted with furin (FIGS. 4I and 4J).

Next, we tested ACE2 expressing cells (FIG. 4K). Considering spike is substantially large plus heavily glycosylated protein and 22-FuG1 is not a commercially established antibody for spike immunoprecipitation, we did not obtain a visibly effective (and clean) spike pulldown by 22-FuG1 on immunoblots. However, it did pull down furin effectively only from spike transfected cells (FIGS. 4L-4N; see also Blumberg et al., 2019). A nonspecific G4S linker with random positive charge distribution in Fc-extended peptide in 22-IgG1 (22-IgG1-RCC) did not pulldown furin (FIGS. 4M and 4N). The latter further strengthens importance of competitive FuG1 peptide being responsible for furin interactions.

Next, we performed IP with another spike-RBD targeting antibody named 6.30-FuG1 (see FIG. 5A). Indeed, 6.30-FuG1 did pull-down detectable S0 spike (and furin) selectively from spike transfected HEK-293 cells (FIG. 4O). The ˜80 kDa (S1/S2 cleaved) fragment was also partially detectable in only from FuG1 immunoprecipitated and spike transfected (FIG. 4O, middle blot) cellular lysates. As both OVCAR-3 cells (FIG. 4J) and HEK-293 cells (FIG. 4O) do not express ACE2 but immunoprecipitated furin along with target antigens (DR5 by KMTR2 and spike by 6.30), respectively, our results support ACE2 co-engagement independent interaction of FuG1 with spike and furin.

### Example 4

### FuG1 Interferes with Optimal S1/S2 Processing of Spike in Cultured Cells

To test FuG1 mediated interference in generating fusion ready S2 fragment, multiple spike RBD and non-RBD targeting antibodies were tested (FIG. 5A, top). We hypothesized endosomal and surface accumulation of spike targeting antibodies before optimal spike expression and shutting to surface upon antibody treatments. HEK-293 cells were treated with 22-FuG1 and other control antibodies 4 hrs post-WT spike (S0) plasmid transfection. A control plasmid with mutations at the spike's S1/S2 furin site (ΔRRAR) was also transfected to confirm the loss of furin cleavage (FIG. 5A, bottom). 24 hours post-transfection (20 hours antibody treatment), lysates were analyzed for ˜80 kDa S2 fragment generation. FuG1 antibody was as effective as ΔRRAR mutant spike (see Johnson et al., 2021) in inhibiting spike cleavage at S1/S2 (FIG. 5B). 22-IgG1 and 22-IgG1 having random charge control distribution in Fc-extended peptides were ineffective. Similar results were evident in ACE2 receptor-expressing HEK-293 (293-ACE2) cells (FIG. 5C), calu3 cells (FIG. 5D), and VERO-E6 cells.

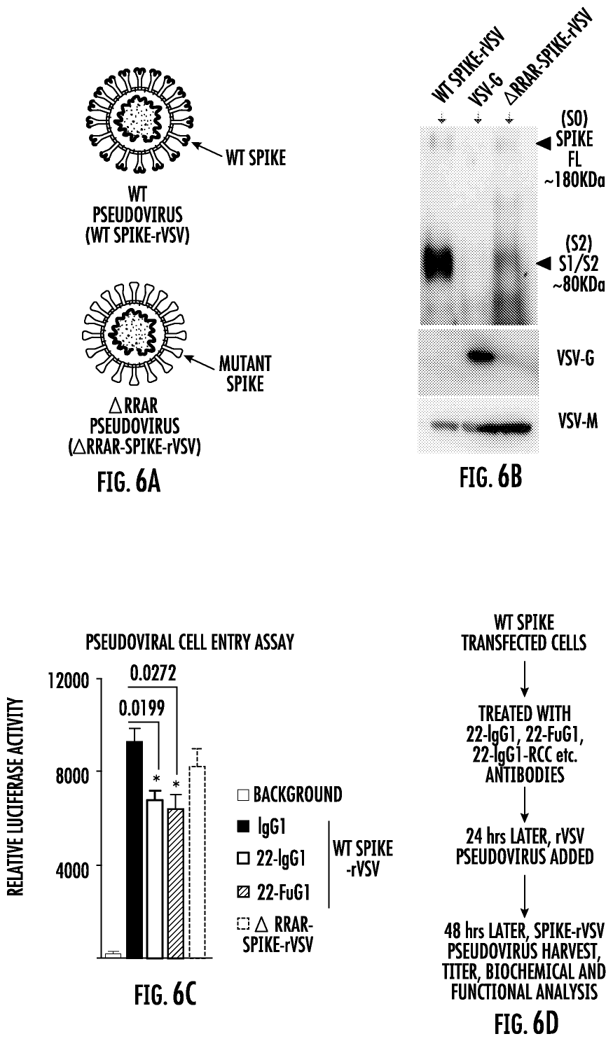

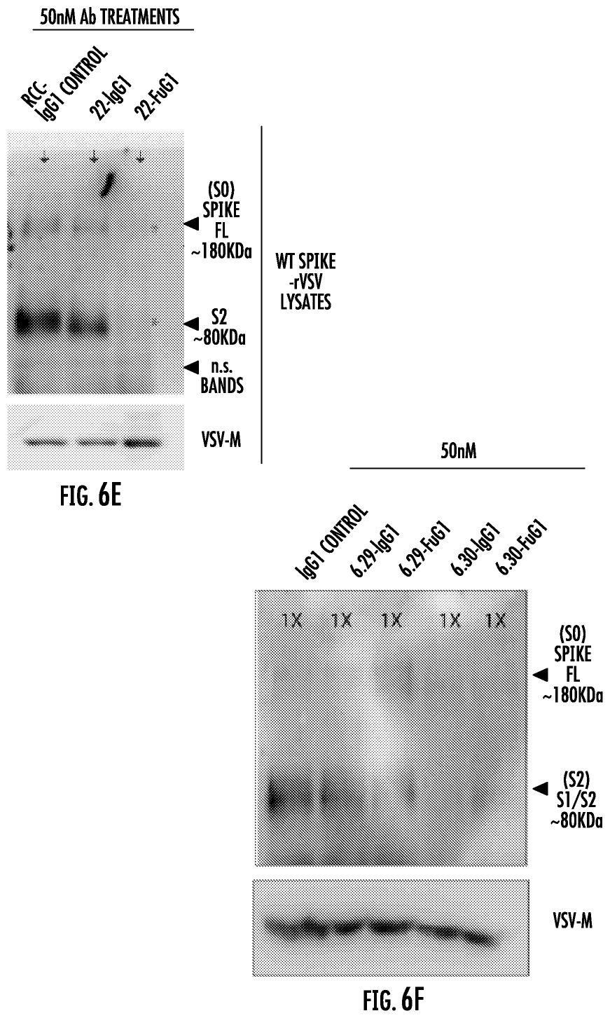

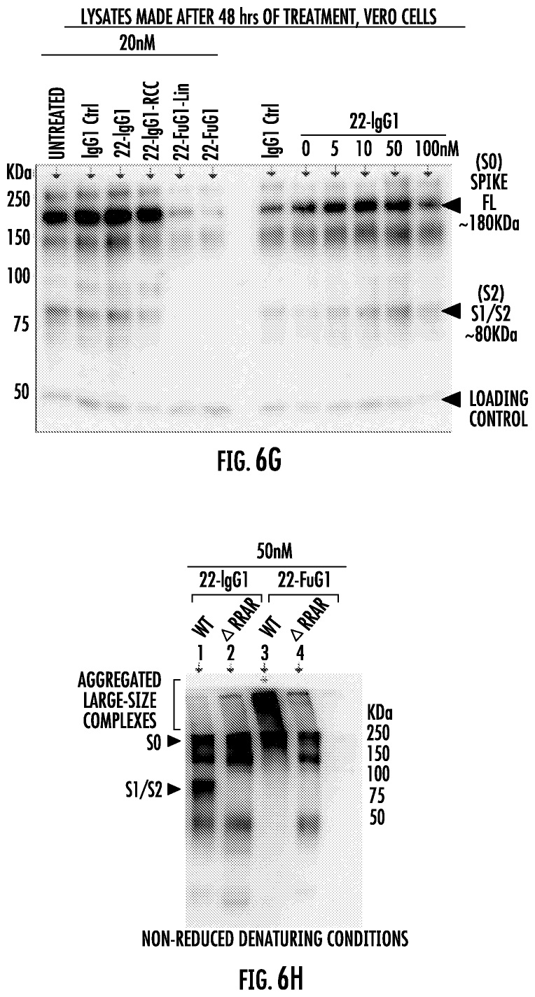

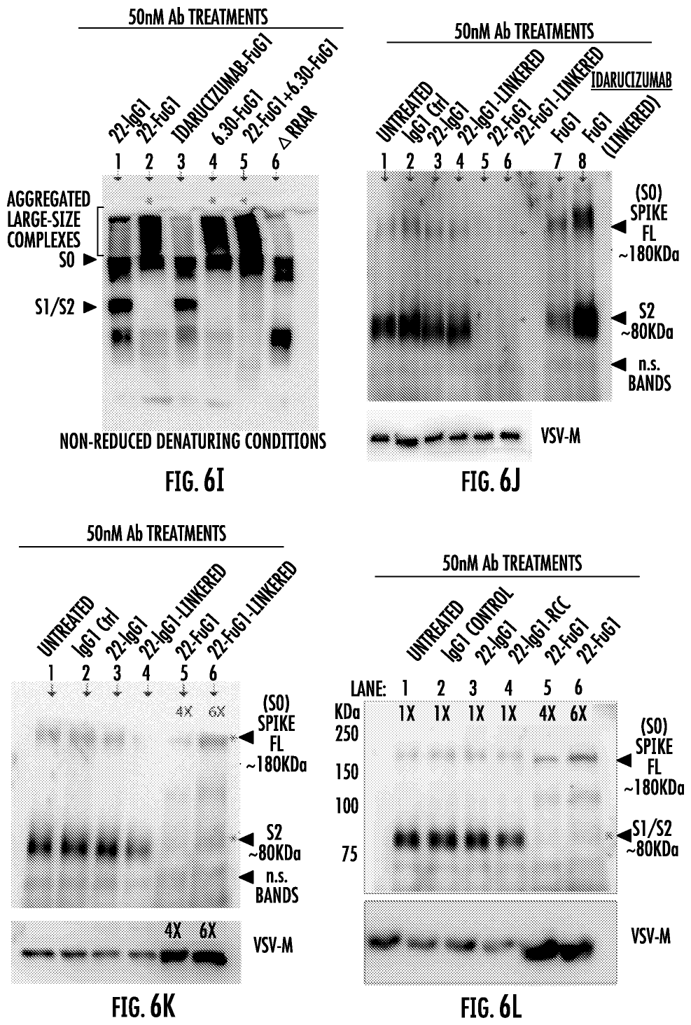

Next, we made use of previously described and established ACE-2-RBD neutralizing (6.29-IgG1, 6.30-IgG1) and non-neutralizing (outside RBD region targeting, 12.19-IgG1, 12.25-IgG1) antibodies (FIG. 5A; see Rogers et al., 2020). When 6.29, 6.30, 12.19, and 12.25 converted in FuG1 antibodies, similar spike S1/S2 cleavage inhibition was observed (FIGS. 5E-5G). These results were consistent with the broad applicability of the FuG1 strategy. We must note that despite being non-inhibitory on spike S1/S2 cleavage, RBD neutralizing 6.29-IgG1 and 6.30-antibodies were significantly more effective than non-neutralizing RBD binding 22-IgG1, and non-RBD binding 12.25-IgG1, and 12.19-IgG1 antibodies in syncytia blockade (FIG. 5H). As syncytia formation requires fusion of spike expressing cells with other spike non-expressing (or expressing) ACE2+ cells, the latter supports the potential implication of the FuG1 strategy on the spike expression in viral-producing cells (see FIG. 6). On the other hand, RBD neutralizing antibodies interfere at the interface of spike-ACE binding (Liu et al., 2020; Rogers et al., 2020). Nonetheless, the conversion of both RBD neutralizing and non-neutralizing antibodies (Rogers et al., 2020) into FuG1 further enhanced their syncytia blockade function. However, it was significantly more evident for non-RBD neutralizing antibodies (FIG. 5H). We saw similar results of syncytia blockade, reduced S1/S2 cleavage, and reduced surface spike expression in VERO-E6 cells in the presence of 22-FuG1 antibody (FIGS. 5I and 5J).

### Example 5

### Pseudovirions Generated in Presence of FuG1 Antibody Contain Inadequately Processed Spike

Replication-restricted VSV particles (G*ΔG-luciferase-rVSV) harboring SARS-CoV-2 spike protein (pseudovirions) have been established to faithfully reflect the key aspects of host cell entry and infection (see e.g., Whitt, 2010; Kleine-Weber et al., 2019; Cantuti-Castelvetri et al., 2020; Hoffmann et al., 2020a; Hoffmann et al., 2020b). Thus, to test FuG1 mediated blockade of viral infections, we generated replication-defective WT and furin mutants (RRAR-GSAA: ΔRRAR) spike expressing luciferase-VSV pseudovirions named rVSV-Spike*ΔG (WT-Spike-rVSV) and rVSV-ΔRRAR-Spike*ΔG (ΔRRAR-Spike-rVSV; FIG. 6A), without any prior antibody treatments as described earlier (see Whitt, 2010; Kleine-Weber et al., 2019; Hoffmann et al., 2020a; Johnson et al., 2021). 48 hrs later, viral supernatants were collected, followed by ultracentrifugation in a sucrose-gradient solution (see Whitt, 2010; Hoffmann et al., 2020a; Johnson et al., 2021). Using immunoblotting, we observed significantly reduced S2 fragment release (spike cleavage inhibition) on ΔRRAR-Spike-rVSV pseudovirions as compared to WT-Spike-rVSV (FIG. 6B) as described in Johnson et al., 2021. VSV-M served as loading control for equal viral lysates in these studies (FIG. 6).

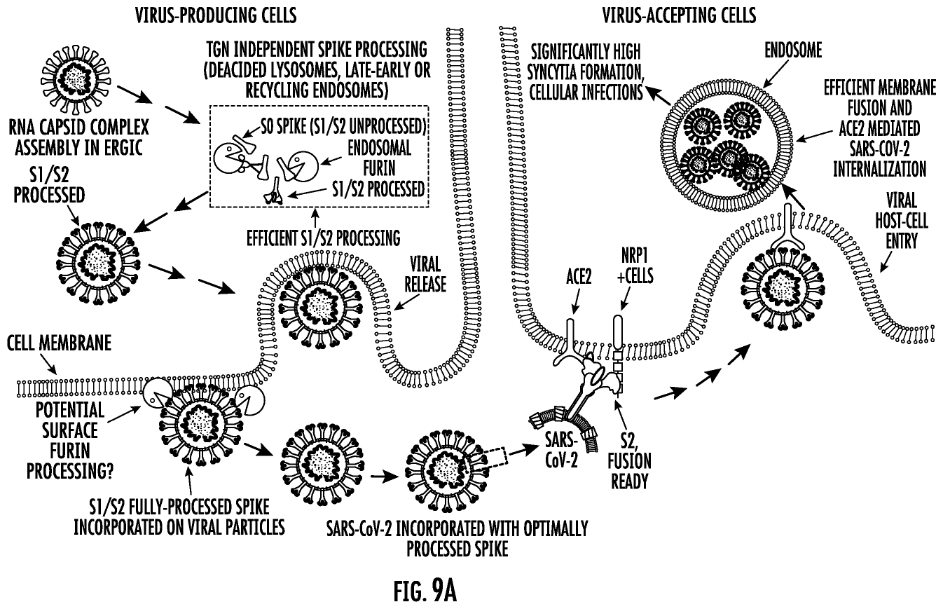

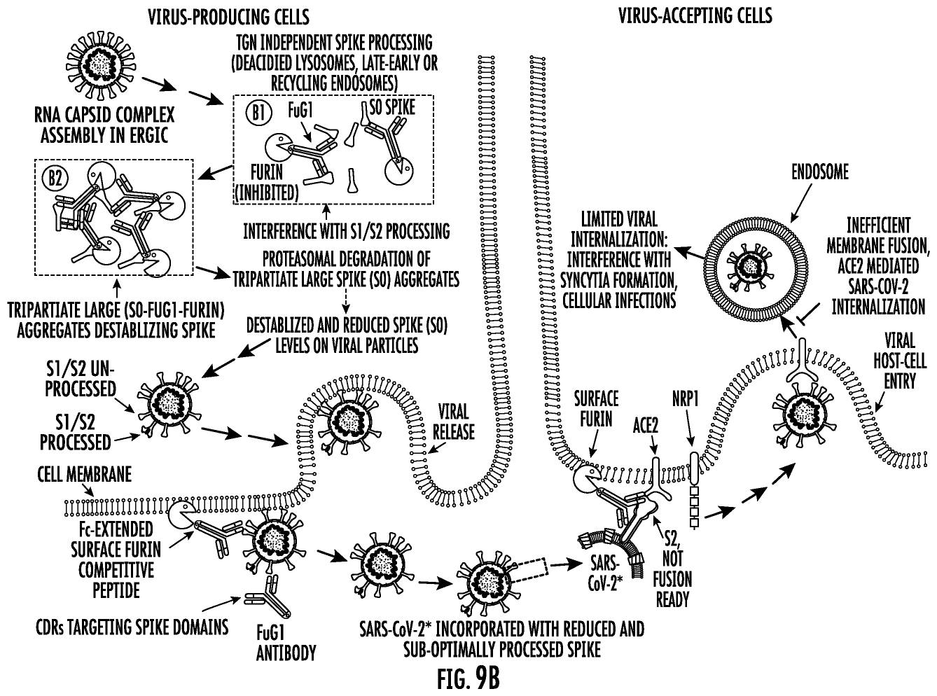

Next, we tested replication-restricted WT spike-bearing luciferase-VSV pseudovirions (WT-Spike-rVSV) in an established assay that uses luciferin readout values as an indicator of host cell entry (Cantuti-Castelvetri et al., 2020; Hoffmann et al., 2020a). WT-Spike-rVSV pseudovirions were generated without prior treatment of antibodies (as described in FIG. 6B), followed by 1 hour incubation with 22-IgG1 and 22-FuG1 (100 μg each), followed by transduction onto 293-ACE2 cells in 96 well plates. 14-16 hours post-transduction, lysates were analyzed for luciferase activity as described in Hoffmann et al., 2020a. 22-FuG1 was only marginally more effective over 22-IgG1, although insignificant (FIG. 6C). These results indicated that a significant S1/S2 furin cleavage event occurred in spike-expressing (and viral producing) cells prior to spike incorporation on replication-defective VSV particles (see FIG. 9A). The reduction in luciferase activity (FIG. 6C) by both 22-IgG1 and 22-FuG1 was due to their RBD binding partial neutralization function, as shown earlier (Yu et al., 2020).

Next, pseudovirions were generated in the presence of FuG1 antibodies (FIG. 6D). To this end, BHK1 (or 293-T) cells were pretreated with IgG1 control, 22-IgG1, 22-FuG1, and 22-IgG1-RCC antibodies 4 hours post spike (S0) transfection (see FIG. 5). 24 hours later, cells were re-treated with corresponding antibodies, and replication-restricted G*ΔG-luciferase-rVSV particles were added to cells (Whitt, 2010). 48 hours later, harvested viral supernatants were analyzed for S0 and S2 fragments using ultracentrifugation followed by immunoblotting where VSV-M protein served as pseudoviral loading control (FIG. 6E). Unexpectedly, 22-FuG1 significantly destabilized pseudoviral spike (S0) along with an almost complete reduction in fusion ready S2 fragment generation, while both 22-IgG1 and 22-IgG1-RCC were largely ineffective (FIG. 6E). Similar S0 destabilization was evident on Spike-rVSV generated in the presence of 6.29-FuG1 and 6.30-FuG1 antibodies (FIG. 6F, last two lanes). Thus, both target (spike) binding and furin interference function of FuG1 antibodies also potentially interfered with optimal particle incorporation and spike (S0) destabilization in virus-producing cells.

### Example 6

### Spike (S0) Destabilization by FuG1 Strategy

Interestingly, similar to destabilized pseudoviral spike (S0), significantly lower levels of full-length S0 protein expression were also evident in FuG1 treated cellular lysates (FIGS. 5B-5G, see S0 band). The latter was despite the similar level of DNA concentrations during spike plasmid transfection (confirmed by mRNA expression analysis). These results collectively suggested additional FuG1-mediated structural destabilization/degradation of full-length S0 spike potentially due to sustained and non-cleavable interactions of furin-FuG1-spike in the tripartite complex. Since the spike transfected cellular lysates were made within 16-20 hours of various antibody treatments (FIGS. 5B-5G), to reassess potential protein destabilization of the spike (S0) protein, we increased the treatment time (>48 hours) of FuG1 antibodies before generating cellular lysates. Strikingly, along with inhibition of S1/S2 cleavage, we also observed a time-dependent reduction in spike S0 levels (FIG. 6G). Neither 22-IgG1 nor the random charge antibodies destabilized the S0 spike.

Recently, spike RBD targeting and in silico engineered Fc-fusions peptides demonstrated robust proteasome-directed spike degradation to inhibit viral loading and production (Chatterjee et al., 2020). Furthermore, the large protein aggregates are routinely fragmented and processed by proteasome complex (Cliffe et al., 2019; Karmon & Ben Aroya, 2019). Since the Fc-fusion-based FuG1 strategy also reduces the overall spike (S0) levels (both cellular transfections and pseudovirions; see FIGS. 5B-5G, 6E, and 6F), we tested the possibility of generation of higher size aggregate complexes on the SDS gel immunoblots. Furthermore, as FuG1 is bivalent, its interactions with more than one tripartite spike-FuG1-furin complex to generate higher size aggregates is a potential possibility for spike destabilization and reduced overall S0 levels in a proteasome-dependent manner. To this end, we carried out non-reducing denaturing gels of FuG1 and other control antibody-treated samples followed by spike immunoblotting. Notably, only FuG1 treated WT spike transfected cellular lysates showed higher aggregated complexes with immunoblotting signal against spike antibody (FIG. 6H, 3rd lane with asterisk). Similar results were evident with another FuG1 (6.30-FuG1) antibody-treated against WT spike transfected cells (FIG. 6I). Strikingly in both the scenarios, the ΔRRAR mutant spike transfected cells did not generate these higher aggregated complexes upon FuG1 treatments (FIGS. 6H and 61). Thus, simultaneous FuG1's variable domain interactions with spike RBD, coupled with FuG1's Fc-peptide interactions spike-furin complex, were critical for spike (S0) instability.

To confirm the potential role of the tripartite spike-FuG1-furin complexes being responsible for the higher aggregate formation and S0 destabilization, we made use of a non-cellular target engaging antibody against the Pradaxa drug (a blood thinner small molecular inhibitor) into FuG1 antibody (also described earlier in FIG. 4O). An anti-Pradexa antibody called idarucizumab (Shivange et al., 2018) has been exclusively used in our lab as a control antibody (Shivange et al., 2018; Mondal et al., 2021a). Since BHK1 cells (or MEM media) or endosomes do not express or contain blood thinner small molecular inhibitor Pradaxa, we hypothesized that due to the lack of spike target binding, the idarucizumab-FuG1 would not destabilize S0. No higher spike aggregates were evident in idarucizumab-FuG1 treated lysates (FIG. 6I, compare lane 3 with lanes 2, 4 and 5 with asterisk on top of gel). When tested, despite similar levels of expression viral assembly matrix protein (VSV-M), significantly reduced levels of spike S0 were loaded on pseudovirions only when generated in the presence of 22-FuG1 antibody but not idarucizumab-FuG1 (FIG. 6J, lanes 5, 6 vs. 7, 8). When 4× and 6× pseudoviral lysates were loaded (evident with VSV-M protein), full-length S0 protein levels were similar to untreated, and IgG1 treated samples (FIG. 6K). Furthermore, even in these conditions, FuG1 antibodies significantly inhibited cleavage at S1/S2 sites, as evident with highly reduced S2 signal (FIG. 6K, compared lanes 1˜4 vs. 5, 6). We reconfirmed these results with the second set of assays in similar conditions (FIG. 6L). Collectively, spike-targeting FuG1 strategy not only competitively inhibited furin S1/S2 cleavage function but also interfered with particle incorporation on viruses in producer cells via destabilizing S0 levels (see FIG. 9).

### Example 7

### Proteasome Inhibition Restores Spike S0 Levels in FuG1-Treated Lysates and Pseudovirions

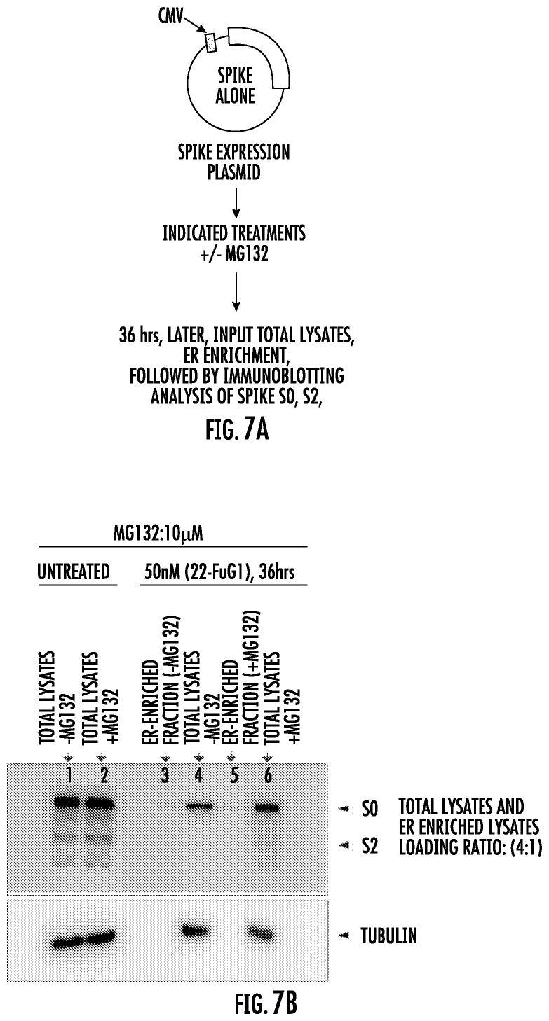

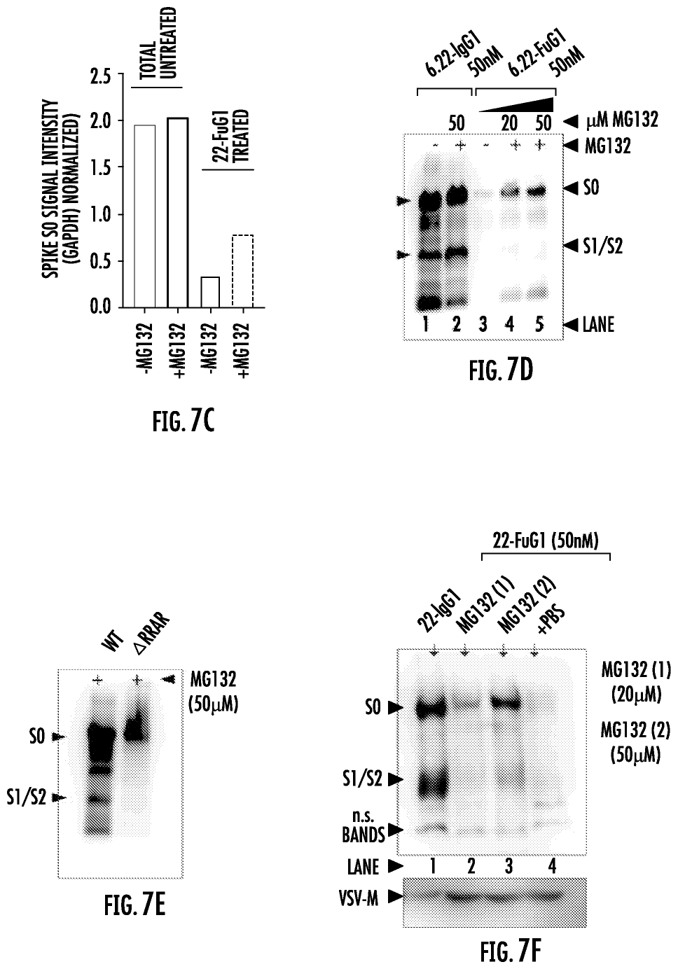

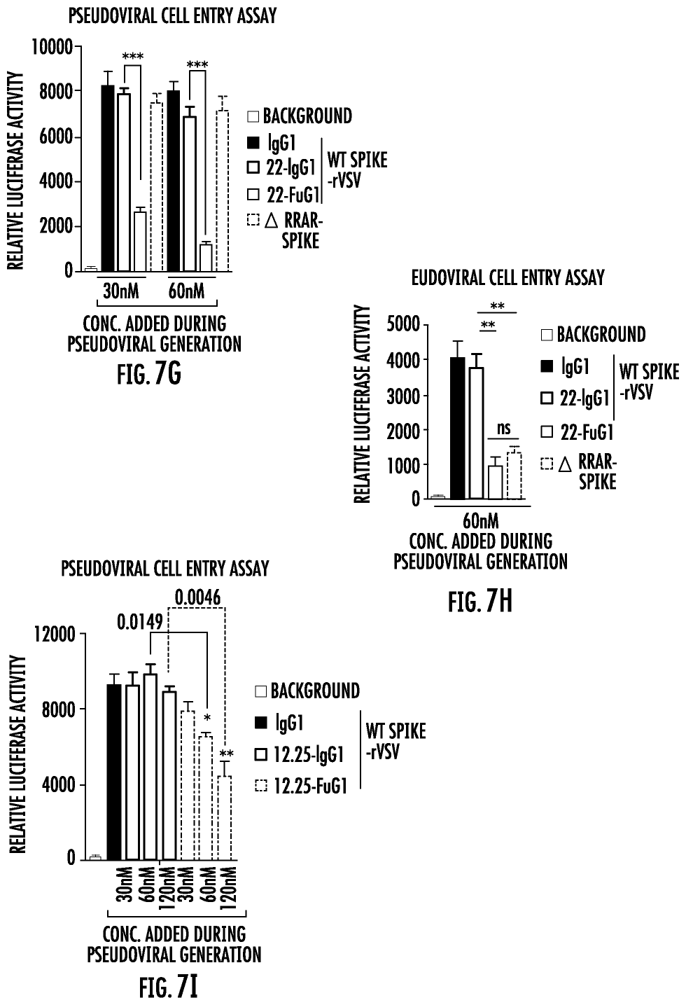

Similar to the published findings of proteasome function mediated spike destabilization by in silico engineered Fc-fusion peptides (Chatterjee et al., 2020), we observed partial rescue of spike S0 loss at 10 μM concentration of proteasome inhibitor concentration MG132 (FIGS. 7A and 7B). When analyzed via subcellular fractionation, preliminary spike S0 signal in ER enriched fraction was very similar, while total spike S0 signal was at least two-fold higher in FuG1 and MG132 treated lysates (FIGS. 7B and 7C). To further confirm these findings, we increased MG132 concentrations (FIG. 7D). Treatment of 50 μM MG132 significantly increased total spike S0 signal, while S1/S2 cleaved fragment was almost invisible (FIG. 7D, compare lanes 2 and 3 vs. lanes 2 and 5). As expected, MG132 alone did not interfere with the furin processing and non-processing of WT and ΔRRAR spike, respectively (FIG. 7E).

To further confirm these results, we generated pseudovirions in the presence of FuG1+MG132 similar to the conditions described earlier (see FIGS. 6E, 6F, and 6J-6L). When harvested viral supernatants were analyzed for S0, and S2 fragments (48 hours later), MG132 treatment rescued spike S0 destabilization in FuG1 treated samples (FIG. 7F, compare lanes, 2, 3 vs. 4). Notably, as expected, S2 fragment generation remained inhibited despite S0 stabilization (FIG. 7F). These results further strengthen the dual working mechanism of the FuG1 strategy to competitively inhibit furin S1/S2 cleavage function and S0 destabilization. Thus, both altered spike confirmation around the S1/S2 region (Johnson et al., 2021) and potentially large aggregates of spike-FuG1-furin interferes with optimal viral transmembrane coat protein assembly.

Next, using 293-ACE2 cells, we tested the effect of reduced S1/S2 cleaved and reduced pseudoviral loaded spike in transduction assays. With luciferase activity as a measure of viral pseudo-infection (host cell entry), WT-Spike-rVSV pseudovirions generated in presence of 22-FuG1 (with increasing concentrations) were significantly less infectious compared to ΔRRAR-Spike-rVSV pseudovirions after 24 hours (FIG. 7G). In support of previous studies, furin mutation did not change cellular entry of ΔRRAR-Spike-rVSV mutant pseudovirions (Hoffmann et al., 2020a). It must be noted that, unlike 22-FuG1, WT-Spike-rVSV pseudovirions generated in the presence of 22-IgG1 gained cellular entry very similar to pseudovirions generated with IgG1 control treatment. Similar results of significantly reduced host cell entry (viral infection) were evident when WT-Spike-rVSV pseudovirions were generated in the presence of other spike targeting antibodies converted into FuG1 antibodies and were tested using TMPRSS2⁻ 293-ACE2 cells (FIG. 7I). Importantly, in support of previous studies (Hoffmann et al., 2020a), ΔRRAR-Spike-rVSV mutant pseudovirions remained limitedly infectious against TMPRSS2⁺ Calu-3 cells (FIG. 7H). These results additionally supported the furin inhibition mediated gain of S0 destabilization and S1/S2 cleavage interference function of FuG1 strategy, which collectively interfered with optimal particle incorporation in infectious particle producing cells during viral egress and subsequent infections (Johnson et al., 2021).

### Example 8

### Spike Targeting Provides Specificity Against Furin Function in FuG1 Strategy

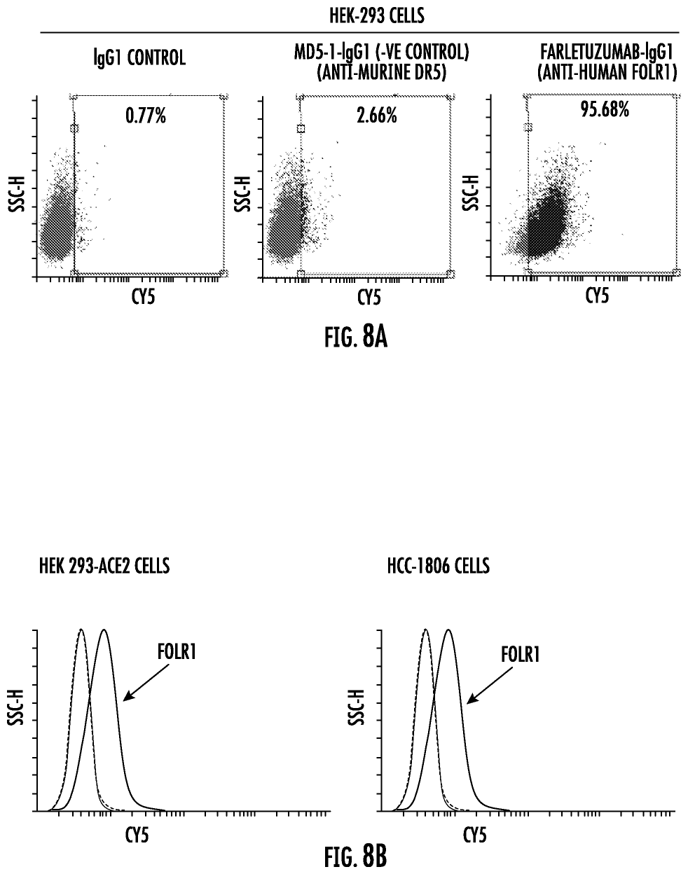

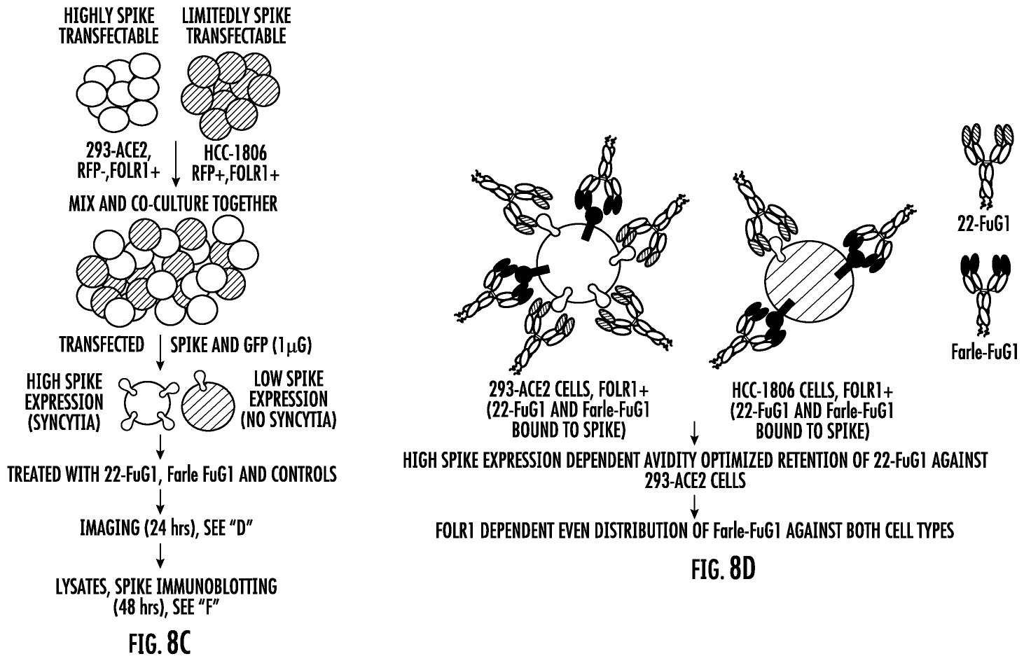

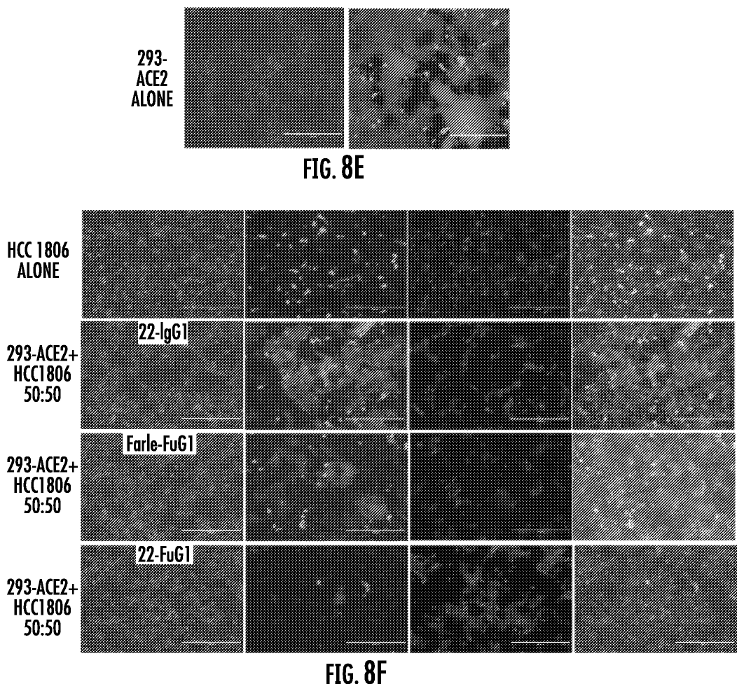

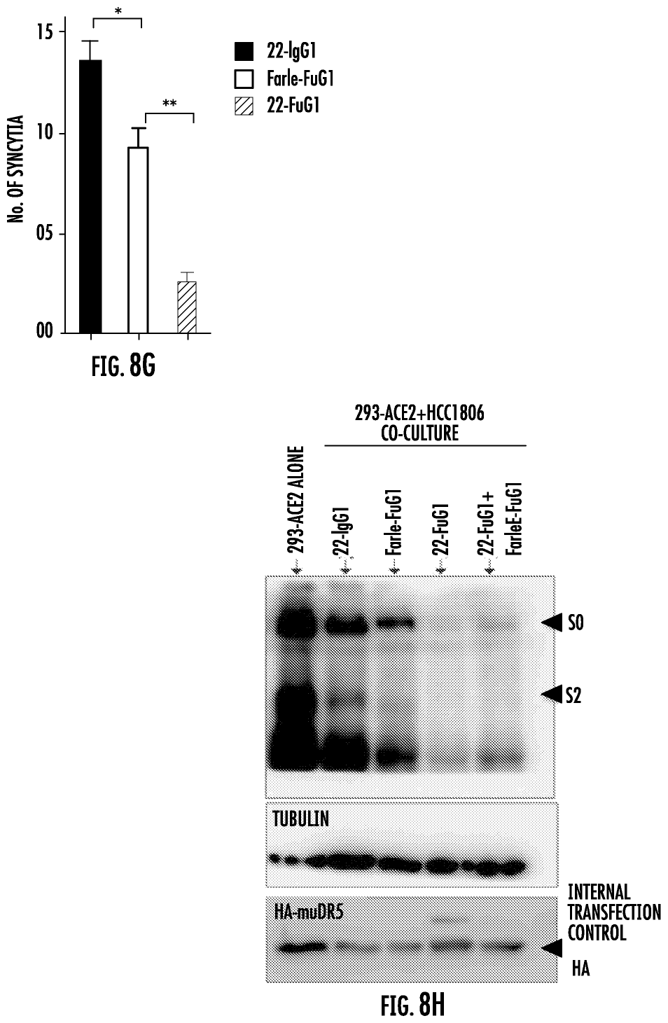

To confirm the FuG1 approach's specificity of furin blockade only against spike-transfected cells, we co-cultured a comparable level of FOLR1 expressing (FIGS. 8A and 8B) 293-ACE2 and RFP-stable HCC-1806 cells (FIG. 8C). Previous studies have established selective detection of only multiple-nucleated enlarged syncytium at low fluorescent magnification (10×) if the spike protein was transfected along with GFP (Xia et al., 2020). As a result, the GFP signal was almost undetectable from non-syncytial mononucleated cells at <10× magnification (Xia et al., 2020). Spike-EGFP transfected cell co-cultures were treated with 100 μg 22-IgG1, farletuzumab-FuG1, and 22-FuG1 antibodies in a manner similar to that described in FIG. 5. The HCC-1806 cells were limitedly transfectable as compared to 293-ACE2, <20% vs >85% based on pcDNA3.1-EGFP alone transgene expression. Thus, we hypothesized equal FOLR1 expression dependent early (or late) endosomal/lysosomal or cell surface occupancy of farletuzumab-FuG1 against both cell-types in cocultures (FIG. 8D). On the contrary, 22-FuG1 would exclusively target and enrich early (or late) endosomal/lysosomal or cell surface compartments against high spike expressing 293-ACE2 cells. The broad distribution of farletuzumab-FuG1 in the coculture assays would inhibit furin function regardless of spike expression. On the contrary, 22-FuG1 would selectively inhibit furin-mediated spike cleavage in target (spike) expressing cells. When tested, farletuzumab-FuG1 only reduced ˜30% syncytia load compared to 22-FuG1 (˜90%) in co-cultures (FIGS. 8E-8G). Although we expected significantly more syncytia (60-70% vs. ˜30%) in farletuzumab-FuG1 treated conditions, the discrepancy could be due to higher cellular stability and recycling function of farletuzumab antibody, which, unlike 22-IgG1, is a humanized clinical antibody with optimal serum stability (Konner et al., 2010). Regardless 22-FuG1 was significantly more effective (FIG. 8G). When various antibody-treated and co-culture cell-generated lysates were analyzed after 48 hours of treatment, farletuzumab-FuG1 only partially reduced S2 conversion in lysates, while 22-FuG1 was highly effective in reducing both S0 and S2 (FIG. 8H). These results collectively supported cellular selectivity toward furin inhibition (and interference with S2 generation) and potential spike destabilization by the FuG1 strategy.

### Example 9

### Antibody Constructs Having Complement Function Interfering and Spike-Directed Furin Cleavage Blocking Functionality

Covid-19, caused by SARS-CoV-2, remains a public health threat worldwide. Although RNA and DNA vector-based vaccine immunization has proven highly effective against hospitalization and mortality, their limitation in blocking the viral transmissibility remains the most significant challenge for the past two years (Puhach et al., 2022). Likewise, pre-clinical and clinical SARS-CoV-2 spike neutralizing antibodies have been highly effective against viral infection and pathogenesis (Razonable et al., 2021). Paradoxically, the super high affinity of SAR-CoV-2 spike neutralizing antibodies renders them ineffective against the viral mutant variants that directly interfere with the binding epitope and spike spatial confirmation (Kozlov, 2021). Thus, the shared limitations and inability of vaccines and antibodies (Richman, 2021) to block viral transmission demand creative and out-of-the-box strategies that decisively target and limit SAR-CoV-2 transmissibility and the chain of the infection cycle.

As the SARS-CoV-2 replication cycle and transmissibility is highly dependent on its spike processing by the host protease system (Johnson et al., 2021), we have recently described host furin interfering and spike-targeting IgG1 antibody named FuG1 (Mondal et al., 2022a). Our comprehensive cellular and pseudoviral testing of the FuG1 strategy strongly support its high applicability in blocking the viral chain of infection and transmissibility (Mondal et al., 2022a). Using primary human lung cell lines, we have also preliminarily confirmed FuG1 antibody-mediated blockade of authentic SAR-CoV-2 (Wuhan strain) replication. In addition, our results support gain in spike destabilization function by FuG1 antibody. The latter further validates the blockade of viral transmissibility by the FuG1 antibody.

Notably, independent of immunization status, recent studies have also described the overactivation of complement pathways in severe SARS-CoV-2 infection (Bosmann, 2021; Afzali et al., 2022). The latter is attributed to adaptative antibody immune response against SARS-CoV-2 and classical antibody-Fc engagement of complement components. Alternatively, highly glycosylated SARS-CoV-2 surface patterns could overamplify the lectin-activated complement pathway (Holter et al., 2020). Significantly, irrespective of the trigger, complement overactivation directly contributes to tissue injury, organ failure, and mortality due to local and systemic inflammation in severe infections). The complement system comprises a cascade of protein cleavage events, ultimately generating a membrane attack complex (MAC) to disrupt cellular breakdown and inflammation.

Both classical and alternate complement activation pathways converge via C3 activation to institute inflammation via C5 cleavage, followed by MAC, cell lysis, and tissue injury. Thus, inhibiting C5 cleavage (into C5a and C5b) in the infected tissue (Zwarthoff et al., 2018) would interfere with complement-mediated hyperactivation. We hypothesize that, unlike systemic administration, the enhanced local delivery of anti-C5 inhibiting antibody will maximize its activity where the utmost inflammation occurs in severe covid-19 patients. Hence to co-target spike protein and complement activation in one single-agent molecule, we propose to engineer a novel complement function interfering and spike-directed furin cleavage blocking bispecific antibody (CI_FuG1). The CI_FuG1 antibody would work via avidity optimized retention against the spike in SARS-CoV-2 infected tissue, enabling the high local concentration in the lung and avoiding the unwanted toxicity of systemic delivery. In addition to high-affinity targeting, spike RBD neutralization (Han et al., 2022), and C5 cleavage inhibition (Robertson et al., 2018), the CI_FuG1 is also inherently designed to interfere with furin protease spike cleavage function to limit the viral chain of transmission.

This Example relates to the following, based on the FuG1 data disclosed elsewhere. A C5 inhibiting and spike RBD co-targeting bispecific FuG1 antibody is provided. In some embodiments, the antibody is referred to as a CI_FuG1 Bispecific antibody. This representative antibody is generated and characterized using SPR BLI studies. This Example also investigates spike binding-dependent furin cleavage inhibition, spike proteasomal destabilization, and inhibition of viral replication by CI_FuG1 antibody. This Example also tests pre-clinical applicability of CI_FuG1 using in vivo SARS-CoV-2 models: testing in vitro and cellular complement (C5 cleavage) interfering function of CI_FuG1 and testing in vivo efficacy of CI_FuG1 using humanized (ACE2 receptor-expressing) mouse model of COVID-19.

With the unpredictability of the emerging SARS-CoV-2 variants (Markov et al., 2022) and prevalent severe prolonged inflammatory infection, Covid-19 remains a global public thread. This Example focuses on generating a safe, multifunctional, and targeted viral strategy to block the SARS-CoV2 chain of the infection cycle. The disclosed approach functions independent of the loss of the SARS-CoV-2 neutralization function with the newer variants. Thus, the CI_FuG1 antibody can have a high therapeutic value to improve the severity of COVID-19 patients, and the platform can apply to other viral pathologies.

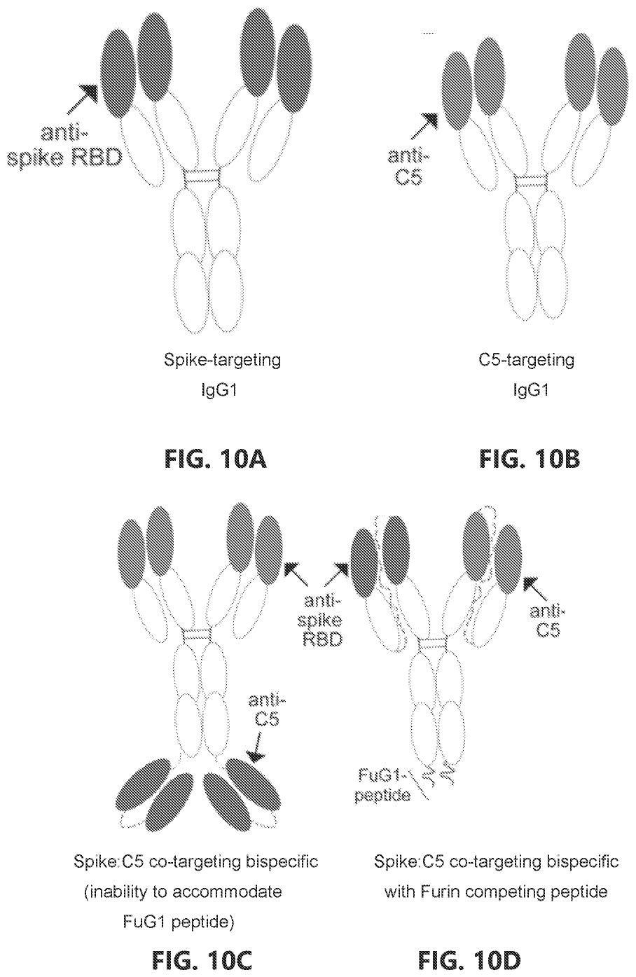

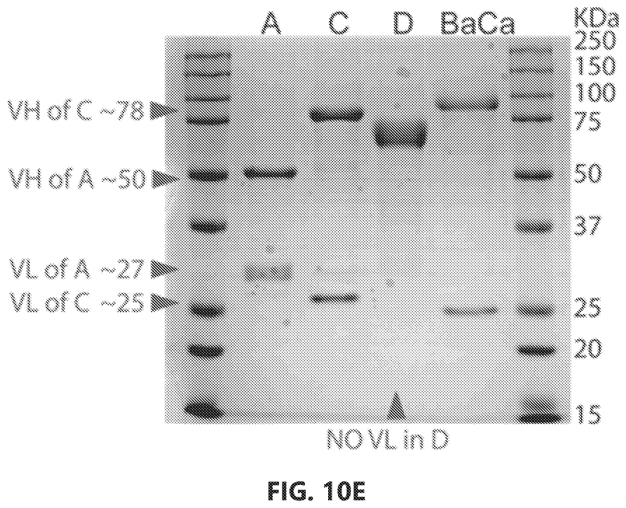

As of Jun. 6, 2022, over 535 million coronaviruses (COVID-19) cases and global deaths have been reported (https://coronavirus.jhu.edu/map.html). SARS-CoV-2 causes COVID-19 (Matheson et al., 2020), and multiple vaccines against the virus have significantly reduced mortality and hospitalization (Mohammed et al., 2022). However, despite a large world population being vaccinated, the pandemic remains global health and financial threat. As vaccines cannot prevent transmission (Eyre et al., 2022), the continuously replicating SARS-CoV-2 could accumulate self-advantageous mutations in its genome to further enhance human transmissibility and immune escape (Read et al., 2015). Thus, if continuous viral transmissibility is kept unchecked, the highly replicating virus could potentially evolve into a serve pathogenic virus in the future (Read et al., 2015; Cho et al., 2021; Cobey et al., 2021). Hence efficient targeting and blockade of SARS-CoV-2 chain of transmission remains the first critically unmet medical need. Second, in addition to unconstrained viral transmissibility, multiple studies have implicated dysregulation and hyperactivation of the innate complement pathways as a hallmark of severe SARS-CoV-2 infections and deadly pathogenesis (Boussier et al., 2022; Savitt et al., 2021; Ma et al., 2021; Santiesteban-Lores et al., 2021; Ramlall et al., 2020). In high-risk patients, the complement system, one of the first lines of the innate host defense, can be activated via classical or alternative pathways upon SARS-CoV-2 infection (Ramlall et al., 2020). Both paths converge on the C3/C5 complexes to generate a pore-forming membrane attack complex (MAC; Thai & Ogata, 2005). The MAC produces holes in the cell membrane, destroying target cells to contribute to a high degree of local and systemic inflammation (hyperinflammatory phase; Scolding et al., 1989). As MAC is dependent on the C5 convertase mediated breakdown of complement protein C5 into C5a and C5b, antibodies and inhibitors targeting C5 cleavage could be of significant importance in overcoming severe COVID-19 pathology (Gorham et al., 2017; Doorduijn et al., 2017). However, the tissue-specific targeting of C5 antibodies in SARS-CoV-2 infected tissues remains challenging. To simultaneously target both a) viral chain and transmission and b) the C5 pathway, This Example tests a dual-specificity spike, furin, and C5 targeting antibody named CI_FuG1 (FIGS. 10A-10E). These studies aim to generate data supporting efficient targeting of SARS-CoV-2 severe pathogenesis.

This EXAMPLE relates to a dual-specificity approach to simultaneously target the viral chain of transmission and complement pathway. In some embodiments, the presently disclosed antibody can significantly change the course of SARS-CoV2 infection pathology and the COVID-19 global threat in general.

As discussed elsewhere herein, SARS-CoV-2 entry and infection of the host cell are dependent on spike receptor-binding domain (RBD) interactions with ACE2 (Jackson et al., 2022). However, before ACE2 binding, if the spike protein is cleaved at its arginine-rich basic residues by host cellular furin protease (34, 35), it significantly enhances the chain of transmission and pathogenicity (Johnson et al., 2021). Indeed, furin inhibitors interfere with the SAR-S—CoV2 infection and replication cycle (Cheng et al., 2020). However, these inhibitors are clinically infeasible due to toxicity associated with random tissue distribution and the interference of numerous normal cellular processes in the body, which are dependent on furin function (Thomas et al., 2002). The presently disclosed subject matter describes the engineering of a unique Fc-extended Furin competing peptide strategy using Spike receptor-binding domain (RBD) targeting antibodies. See FIGS. 1C, 1D, and 1E, discussed herein above. This approach is referred herein in some instances as FuG1 (Mondal et al., 2022a).

Referring to EXAMPLES 1-8, using protein biochemistry studies, cellular assays, and a pseudoviral model, our findings support the inhibition of spike cleavage by furin and interference with syncytia generation in cellular and pseudoviral models. In addition, as discussed elsewhere herein, including EXAMPLES 1-8, an unexpected new function of overall spike protein destabilization (and proteasome-mediated degradation) by the FuG1 strategy has been discovered. The latter is due to higher-order furin-FuG1-spike aggregates (see FIGS. 9A and 9B, discussed herein above). Collectively FuG1 antibody has a significant potential to target the viral chain of transmission cycle (see FIGS. 9A and 9B, discussed herein above).

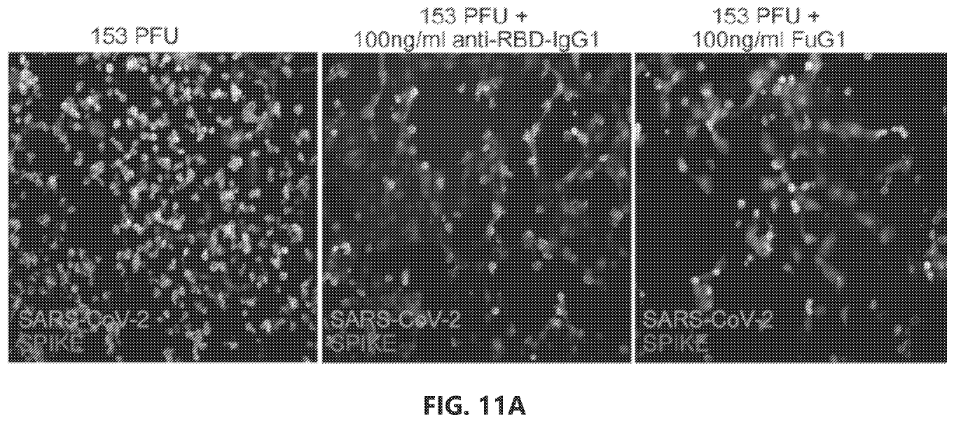

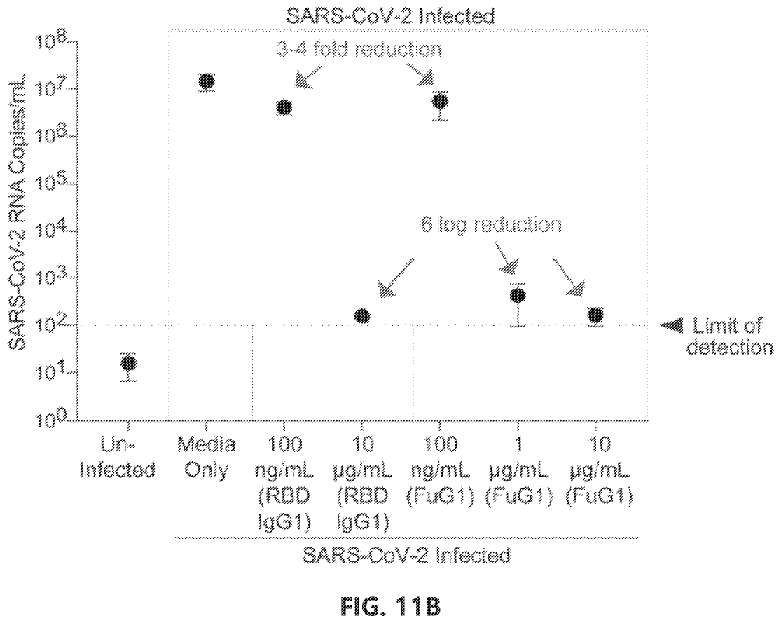

Further, the efficacy of FuG1 in suppressing authentic SARS-CoV-2 infection using Vero E6 cells was evaluated. Cells were infected with 153 PFU of mNeonGreen SARS-CoV-2 (Wuhan stain) for 6 hours, followed by treatment with FuG1 or IgG antibodies. Untreated cells showed robust infection levels, as the green fluorescent infection signal was readily detected under the microscope on day two post-infection. In comparison, both RBD neutralizing IgG and FuG1 at 100 ng/ml dramatically inhibited the SARS-CoV-2 infection on day 4, indicated by a pronounced reduction in SARS-CoV-2 infection signals of mNeonGreen. Moreover, at 10 μg/mL concentration, FuG1 and RBD neutralizing IgG demonstrated total inhibition of SARS-CoV-2 infection on day 4, compared to untreated infected controls. Evaluation of virally infected cells and viral spike protein was performed in fluorescent immunostaining. Cells were stained with anti-SARS-CoV-2 spike antibodies (red when shown in color), and infection was indicated by mNeonGreen fluorescence (green when shown in color). Fluorescent imaging revealed a significant reduction in mNeonGreen signals and decreased distribution of spike proteins in Vero E6 cells treated with 100 ng/ml FuG1 and IgG (FIG. 11A). Moreover, almost no spike and viral infection signals were observed in Vero E6 cells treated with 1 and 10 μg/mL FuG1, while IgG1 needed 10-100 μg/mL concentration (FIG. 11A).

Next, Vero E6 cells and culture supernatant were fixed and processed in the BSL2 laboratory for subsequent assays. Real-time PCR assays evaluated SARS-CoV-2 viral RNA output. Treatment with 100 ng/mL FuG1 and IgG resulted in a 3-4 fold reduction in viral RNA levels compared to untreated samples. Notably, treatment with 10 μg/mL FuG1 and IgG demonstrated significant 6-log reductions in RNA copies of Vero E6 cells, which are close to the levels of detection limit per our real-time PCR assays (FIG. 11B). Once again, only 1 μg/mL of FuG1 effectively eliminated viral load close to the level of detection (FIG. 11B). Our data demonstrated consistent and reproducible suppression in SARS-CoV-2 infection and propagation rendered by FuG1 with even non-RBD neutralizing antibodies (Mondal et al., 2022a).

Engineering a novel C5 inhibiting and spike RBD co-targeting bispecific FuG1 antibody. A complement and spike co-targeting bispecific FuG1 antibody is engineered to target C5 cleavage function in spike dependent manner. The latter maintains the highest anti-complementary activity in the vicinity of SARS-CoV-2 infected tissue rather than systemically.

Bispecific antibody generation and binding Characterization using BLI SPR studies. To generate Spike RBD and C5 targeting dual-specificity CI_FuG1 antibody (FIG. 10D), clinical complement component C5 targeting antibodies were used, which are established to inhibit C5 cleavage into C5a and C5b and terminal MAC formation. The CI_FuG1 antibody configuration resembles an IgG1 and is similar to CrossMab antibodies of Genentech. In this configuration, the affinities against spike RBD and C5 are monovalent (these are variable domains, blue and red when shown in color).

CI_FuG1 was engineered by making use of the following changes. A) knob/hole mutations in CH3-Fc allow heterodimerization of two IgG chains that only differ in the Fv domain (Ridgway et al., 1996); B) Genetically linked glycine-serine linkers (45 GS) between the 3′ end of c-kappa and 5′ end of VH for proper light chain pairing (Shivange et al., 2018; FIG. 10D; see also FIGS. 10A-10C control schematics). Therefore, when run in reducing conditions, the CI_FuG1 antibody showed a single band of ˜75 kDa, as light chain and heavy chain are genetically linked by GS linkers (FIG. 10E). Hence for the studies of this EXAMPLE, anti-C5 antibodies: ravulizumab (Rondeau et al., 2020) and eculizumab (Jodele et al., 2020) were used in preparing the antibody construct. The sequences of ravulizumab (SEQ ID NO: 65) and eculizumab (SEQ ID NO: 66) are available at https://www.rcsb.org/, which is retrieved and synthesized as gene strings, followed by cloning and grafting onto the IgG1 framework as described earlier (Shivange et al., 2018). Next, in vitro binding and activities of both ravulizumab and eculizumab are confirmed. The latter follows engineering of them with knob-into-hole bispecific format (Shivange et al., 2018; Mondal et al., 2021b) in multiple combinations with the spike RBD targeting antibodies such as CR3022, 6.30, and imdevimab, etc. as described elsewhere herein. The rationale for the knob-into-hole bispecific format is ideal for efficient Fc-extended FuG1 peptide engineering (FIG. 10D). Various bispecific combinations are confirmed for expression and simultaneous binding to both spike RBD and C5 using ELISA, BLI, and SPR studies. His and IgG-Fc tagged Spike RDB and C5 protein are used for binding studies. The lead bispecific molecule is selected based on the following representative criteria; 1) expression yield in mg/liter, 2) spike binding dependent furin and complement blockade function, 3) high-affinity in vitro binding to dual antigens, and 4) and overall stability in various freeze-thaw cycles as described earlier (Shivange et al., Cancer Cell. 2018; 34 (2): 331-45 e11).

Confirmation of CI_FuG1 antibody's multifunctionality using spike cleavage inhibition and proteasomal destabilization studies, along with the original FuG1 antibody. As shown elsewhere herein, the FuG1 antibody is highly efficient in interfering with spike furin cleavage to inhibit spike fusion ready S2 fragment generation. To confirm that the RBD binding and furin inhibition is not lost after the bispecific antibody conversion, human lung cells transfected with spike expression plasmid are tested for S2 fragment generation. Bispecific antibodies without Fc-extended FuG1 peptide will serve as a control, and previously described RBD FuG1 is a positive control. Multiple cell lines are tested for the blockade of spike cleavage for rigor and reproducibility. In addition, the authentic SARS-CoV-2 virus, Wuhan strain, and US delta strains can be used to confirm the inhibition of spike cleavage, syncytia blockade, and interference with the viral infection cycle as described earlier. Similarly, overall cellular and viral spike destabilization is tested by analyzing whole-cell lysates and proteasome inhibitor studies as described elsewhere herein. To confirm the CI_FuG1 antibody's ability to destabilize overall spike protein levels in virally infected cells, more than one proteasome inhibitor, such as MG132 and bortezomib, is used as described earlier (Park et al., Transl Res. 2018; 198:1-16. Epub 2018 Apr. 15).

We anticipate that engineering (FIGS. 10A-10E), expression, and binding studies of CI_FuG1 are straightforward. The studies use preexisting antibody engineering, expression, binding assays, protocols, etc., that have been published. Similarly, assays for spike cleavage and spike destabilization are described elsewhere herein. Therefore, the proposed studies are straightforward. However, if experience unexpected results with a particular antibody's IgG1 conversion into CI_FuG1, multiple different spike RBD targeting and C5 inhibiting antibodies are used, including murine C5 specific antibodies (see below). In terms of consistency, rigor, and reproducibility of data by the CI_FuG1 studies, multiple cell lines will be tested for proposed experiments. In terms of reagents for western blotting, immunofluorescence, binding studies, etc., are repeated with more than two different commercial antibodies, kits, or cell lines or assays (such as BLI and SPR). The significance of the differences between the treated versus the vehicle group is determined using 1-way ANOVA (Graphpad Prism). Categoric variables are analyzed with the Fisher exact test. The statistical significance is determined using Mann-Whitney tests with a Bonferroni adjustment for multiple comparisons.

Testing pre-clinical applicability of CI_FuG1 using in vivo SARS-CoV-2 models. The pre-clinical applicability of the newly engineered CI_FuG1 antibody to inhibit complement activation and MAC formation is tested. In addition, the studies establish if CI-FuG1 is effective over complement only targeting or Spike RBD only neutralizing monospecific antibodies. If successful in murine models, the studies set a precedent for potential more significant application of the described approach to target the hyperactivation of complement in serve COVID-19 patients rationally.

To Test in vitro and cellular complement (C5 cleavage) interfering function of CI_FuG1. The complement system remains an effective host defense against various infections, including SARS-CoV-2 (Afzali et al., 2022). However, in multiple severe COVID-19 infection studies, the hyperinflammatory reaction upon complement activation outweighs the beneficial host effects of the complement system). Indeed, severe COVID-19 cases experience pneumonitis, systemic sepsis, organ failure, and death due to complement hyperactivation (Holter et al., 2020; Ma et al., 2021; Rittirsch et al., 2012). The latter directly supports the critical medical need for complement blocking interventions (Santiesteban-Lores et al., 2021). As stated above, the complement system comprises a cascade of protein cleavage events. The ultimate cleavage terminates at C5 to generate C5a and C5b by C3b (known as C5 convertase). C5b is key to forming MAC to instigate cellular injury and tissue destruction. Hence, studies focus on C5 cleavage inhibition by CI_FuG1. As a positive control for these experiments, a commercially available inhibitor known as complement C5-IN-1 (Compound 7; Fattizzo et al., 2020) is uses. C5-IN-1 is a highly efficient small-molecule inhibitor of C5 cleavage and has been established to prevent PNH hemolysis in vitro. In addition, for positive control, clinical monoclonal antibodies such as crovalimab and pozelimab are used along with the bispecific CI_FuG1 antibody (Zelek et al., 2020b). 50% hemolytic complement (CH50) activity assays are carried out to investigate the lysis of sheep red blood cells (SRBC). SRBC is pre-coated with rabbit anti-sheep red blood cell antibody (hemolysin) with CI_FuG1. In a separate experiment, side by side, C5 inhibitory positive and negative control are used as described earlier (Costabile, 2010). To confirm the reproducibility of results with CI_FuG1, blockade of complement-mediated SRBC hemolysis in the presence of normal human and fetal bovine serum (Latuszek et al., 2020) is tested. For these studies, the serum is supplemented with the recombinant WT C5. The complement-mediated hemolysis requires cleavage of C5 into C5a and C5b (which leads to the assembly of MAC). Hence, as described earlier, both C5 and C5a levels are analyzed during hemolysis assays using ELISA and western blotting protocols (Latuszek et al., 2020). Next, there is a focus on studies to confirm that SARS-CoV-2 spike binding does not interfere with the complement blocking function of CI_FuG1. For the latter, added is a recombinant spike RBD domain to the assay mix. Alternatively, recombinant SARS-CoV-2 spike expressing HEK cells mixed with SRBC in the human or fetal bovine serum is used. WT HEK cells serve as a control for these assays. These results collectively confirm the blockade of C5 cleavage by the dual-specificity CI_FuG1 targeting both spike and C5, similar to clinical monospecific antibodies.

Testing in vivo efficacy of CI_FuG1 using humanized (ACE2 receptor-expressing) mouse model of SARS-CoV-2. Multiple humanized mouse adaptive COVID-19 models have been described (Sefik et al., 2021; Dinnon, 2020; Iwata-Yoshikawa et al., 2022). Unfortunately, no humanized C5 and humanized ACE2 co-expressing mouse model is available for testing severe COVID-19 complement-mediated hyperactivation. In addition, most clinical C5 antibodies are not cross-reactive to primate and murine C5; hence remain untested. Indeed, most of them have moved to clinical trials because of high-efficiency C5 cleavage inhibition studies in vitro and ex vivo. Fortunately, one murine C5 cross-reactive antibody (named BB5.1) has been described and whose sequence is available in a public database (Zelek et al., 2020a). Notable BB5.1 efficiently inhibits C5 in mouse serum and prevents C5 cleavage and C5a generation (Zelek et al., 2020a). Hence, for in vivo studies, BB5.1 is engineered with the spike RBD antibody into CI_FuG1. The humanized ACE2+ mouse model (B6J.Cg.Tg K18-hACE2) described by Weiss et al., 2021.

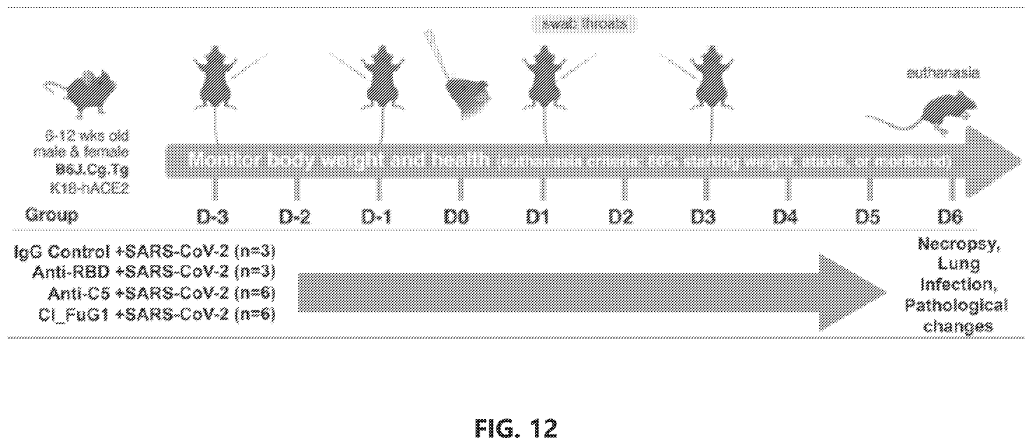

To evaluate the anti-complement function of CI_FuG1 against SARS-CoV-2 infection, the humanized K18-hACE2 mice is treated and administered once daily at 3 days and 1 day before challenge (−D3,−D1 in FIG. 12) and 1 day and 3 days post virus challenge (D1, D3 in FIG. 7), where study day 0 is SARS-CoV-2 inoculation (FIG. 12). Mice are anesthetized with isoflurane for treatment administration. Compounds are be administered with an appropriately sized needle via a subcutaneous route. On day 0, inoculate mice with 104 PFU (in 30 μl DPBS) of SARS-CoV-2-8 (B.1.617.2). body weight is monitored and health for euthanasia criteria: 80% starting weight, ataxia, or moribund. The throat swabs are collected from days 1 & 2, and blood, lung, and other tissue will be examined upon necropsy (FIG. 12, D6). IHC studies of the lung, liver, and heart for anti-hyper complement activation analysis and safety studies are done. These results will confirm the enrichment of CI_FuG1 antibody and significant inhibition of C5a and MAC generation in the SARS-CoV-2 infected lung tissue compared to anti-RBD only and anti-C5 only treated animals.

Assay kits for 50% hemolytic complement (CH50) activity are available commercially from multiple resources. For rigor and reproducibility, sheep red blood cells (SRBC) pre-coated with rabbit anti-sheep red blood cell antibody are purchased from 3 independent vendors. Testing CI_FuG1 with authentic SARS-CoV-2 in human lung cell lines is carried out and humanized ACE2+ mice studies are straightforward as those protocols are standard and published (see e.g., Weiss et al., 2021).

However, in some instances, the conversion of specific IgG1 antibodies into a bispecific format (specifically knob-into-holes) could result in the loss of some activity. Suppose the latter is experienced (upon SPR binding and hemolysis assay data) with murine C5 cross-reactive antibody (BB5.1). In that case, BB5.1 is engineered into a traditional alternate bispecific format without the knobs into holes (See FIG. 1C). No IgG1 conversion has lost activity in the alternate bispecific format (FIG. 1C). The latter is because the two variable Fab domains against two different antigens are significantly far apart (>150 Å) (58) in the alternate bispecific format (FIG. 2C) as compared to knob into holes without the complexity of c-kappa-VH connecting flexible linkers. Thus, if experiencing an issue in BB5.1 conversion into CI_FuG1, the spike RBD and C5 inhibiting bispecific antibody are tested without the furin competitive function. The latter also generates the conclusive results of spike and complement co-targeting in severe SARS-CoV-2 infection. Sample size justification: With ten mice per group, 80% power at the 0.05 level of significance (two-sided) t-test is provided. The significance of the differences between the treated groups is determined using 1-way ANOVA (Graphpad Prism). Categorie variables are analyzed with the Fisher exact test. The statistical significance of viral growth inhibition and pathology reduction are determined using Mann-Whitney tests with a Bonferroni adjustment for comparisons.

### Discussion of the Examples

Disclosed herein in some embodiments is a targeted antibody-based plug-and-play FuG1 strategy that not only directly interferes with the furin-dependent proteolytic mechanism of spike cleavage and activation but also destabilizes the full-length spike protein. Both of these mechanisms collectively interfere with the optimal spike incorporation during viral assembly in producing cells with the potential to impede with SARS-CoV-2 chain of infection cycle (FIG. 9). Considering that antibody-based targeting of intracellular proteins (such as those residing with in ER, or TGN network) requires intracellularly expressed intrabodies (Lum & Tushir-Singh, 2021), FuG1 strategy-mediated blockade of spike S1/S2 furin cleavage support TGN independent spike processing. Nonetheless, the regulatory role of furin in the endosomal network and cell surface is well established (Thomas, 2002). Importantly, both endosomes and cell membranes are a well-defined route of therapeutic antibodies in complex with target-antigen or neonatal Fc receptor (Fc-Rn) based recycling mechanisms (Blumberg et al., 2019; Saunders, 2019). Thus, FuG1 strategy-mediated blockade of furin-driven spike S1/S2 cleavage in the endosomal network of viral-producing cells is consistent with published studies (Lu et al., 2006; Ghosh et al., 2020). Furthermore, the described organelle fractionation studies additionally strengthen the furin-mediated spike processing and viral egress via the deacidified lysosomal/endosomal system (Ghosh et al., 2020). While it is possible that lysosomal deacidification may be due to the overloading of highly glycosylated spike protein or perturbation of ion-channel pumps in the membrane (Lu et al., 2006), it remains to be demonstrated.

Spike-rVSV particles generated even in the presence of furin inhibiting FuG1 antibodies were able to maintain close to ˜15-40% pseudo-infection (depending on the spike targeting FuG1 used), as evident with the host cell entry assays (FIGS. 7G-7I). The latter could be attributed to multiple independent working mechanisms. First, FuG1 antibodies did not completely inhibit pseudoviral S2 generation (FIGS. 6-8), which suggests further optimization of the FuG1 peptide. Second, various SARS-CoV-2 neutralizing or non-neutralizing antibodies fall into multiple categories based on their interactions with spike RBD or non-RBD regions (Blumberg et al., 2019). For example, despite no effect on spike S0 to S2 conversion, unlike 12.25 IgG1, 12.19 IgG1, and 22-IgG1 antibodies, core-RBD targeting 6.30 or 6.29-IgG1 antibodies (Yu et al., 2020) were significantly effective in syncytia blockade (see FIG. 5H). On the other hand, 22-FuG1 was as effective as 6.30 or 6.29-FuG1 antibodies in suppressing spike S0 to S2 cleavage. Thus, the disparity in syncytia blockade was not an optimal predictor of an effective IgG1 conversion into FuG1. In addition, the RBD domain of spike undergoes hinge-like movements in “up” “down” conformations, representing ACE2-accessible or inaccessible states, respectively (Kalathiya et al., 2020; Yuan et al., 2020). Considering the unpredictable interactions of SARS-CoV-2 neutralizing and non-neutralizing antibodies with RBD up and down confirmations (Blumberg et al., 2019; Yuan et al., 2020), defining a particular epitope on the spike for maximum accessibility of FuG1 peptide to competitively saturate furin at “RRAR” sites can be relevant for improving the targeting strategy further (Kalathiya et al., 2020; Lan et al., 2020; McCallum et al., 2020).

Third, previous reports (Hoffmann et al., 2020b) have also described the potential furin independent spike priming by cysteine proteases such as cathepsin B/L (CatB/L) for aiding the infection in potential TMPRSS2⁻ cells. In support, S1/S2 mutant ΔRRAR pseudoviral rVSV-Spike particles gained effective entry in the transduction experiment in TMPRSS2⁻ cells but not in TMPRSS2⁺ cells (FIGS. 7G-71). Considering that the natural SARS-CoV-2 virus with furin mutations was partially infectious in murine models (Johnson et al., 2021), the combinatorial targeting of FuG1 with TMPRSS2 and CatB/L inhibitors is expected to weaken the viral pathogenicity further. Regardless, additional testing is needed in relevant infectious animal models.

Other than furin-targeting small molecular inhibitors, several studies have described innovatively engineered ACE2 trap (Glasgow et al., 2020) and mini protein-based peptide designs (Cao et al., 2020) to neutralize SAR-CoV-2 infection potentially. Unfortunately, many of these agile approaches lack targeting specificity toward an infected cell type. Thus, they are highly likely to inhibit cellular protease function randomly to induce toxicity (10). On the other hand, the described FuG1 approach potentially gives spike target mediated specificity over-described furin/TMPRSS2 protease network targeting small molecule inhibitors and innovative protein-based designs (Cao et al., 2020; Cheng et al., 2020; Glasgow et al., 2020). Furthermore, other dual-specific antibody-based FuG1 strategies co-targeting lung enriched antigens could be engineered on the described foundation for additional enhanced specificity to infected lung cells and tissue (Shivange et al., 2018). Besides, since the FuG1 strategy is only dependent on exploiting spike protein as a targeting anchor to interfere with fusion ready S2 fragment generation in viral producing cells (FIG. 9) if the target antibody binding site is not affected by naturally evolving mutations in SARS-CoV-2 variants (Delta, Omicron etc.), the strategy could potentially be broadly applicable (Villoutreix et al., 2021; Wang et al., 2021). Particularly, the FuG1 strategy has a crucial advantage over other protease targeting strategies in terms of specificity.

Interestingly, earlier reports have described higher incorporation of ΔRRAR mutant spike glycoprotein (higher particular/PFU ratio) on the infectious SARS-CoV-2 virus than WT spike (Johnson et al., 2021). Strikingly, FuG1-targeted and furin competing WT spike shown reduced stability and particle incorporation. The results presented herein provide evidence for the observed disparity of viral incorporation between ΔRRAR mutant spike vs. FuG1-targeted WT spike glycoprotein. Unlike WT spike, ΔRRAR mutant spike is not a furin substrate (Johnson et al., 2021), and hence did not generate slow migrating higher size FuG1-spike-furin aggregates (FIGS. 6H and 61). In contrast, WT spike induces higher size FuG1-spike-furin aggregates. Hypothetically, the latter might be due to the lower furin dissociation rate of FuG1 (FIG. 2C), resulting in the formation of larger multivalent tripartite spike-FuG1-furin complexes. In support of previous studies (Chatterjee et al., 2020), such aggregates destabilize spike protein in a proteasome activation-dependent manner. Regardless, the presently disclosed results support the published studies of spike destabilization by Fc-fusion peptides and furin-mediated events critical for SAR—S—CoV-2 pathogenesis upon cellular entry (Chatterjee et al., 2020; Johnson et al., 2021).

In summary, in some embodiments, the FuG1 approach represents a rational and plug-n-play Fc-extended peptide-based SARS-CoV-2 targeting design, with added potential against the future generations of coronaviridae family and/or other viruses that exploit cellular proteases for deadly infections.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Afzali et al. (2022) The state of complement in COVID-19. Nat Rev Immunol 22 (2): 77-84.
Altschul et al. (1990a) Basic local alignment search tool. J Mol Biol 215:403-410.
Altschul et al. (1990b) Protein database searches for multiple alignments. Proc Natl Acad Sci USA 87:14:5509-5513.
Altschul et al. (1997) Gapped BLAST and PSI-BLAST: a new generation of protein database search programs. Nucleic Acids Res 25:3389-3402.
Barbas (1995) Synthetic human antibodies. Nature Medicine 1:837-839.
Bird et al. (1988) Single-chain antigen-binding proteins. Science 242:423-426.
Blumberg et al. (2019) Blocking FcRn in humans reduces circulating IgG levels and inhibits IgG immune complex-mediated immune responses. Sci Adv 5: eaax9586.
Boesecke & Cooper (2008) Toxicity of HIV protease inhibitors: clinical considerations. Curr Opin HIV AIDS 3:653-659.
Bosmann (2021) Complement control for COVID-19. Sci Immunol 6 (59): eabj 1014.
Boussier et al. (2022) Severe COVID-19 is associated with hyperactivation of the alternative complement pathway. J Allergy Clin Immun 149 (2): 550-556.
Brennan et al. (1985) Preparation of bispecific antibodies by chemical recombination of monoclonal immunoglobulin G1 fragments. Science 229:81-83.
Burton & Barbas (1994) Human antibodies from combinatorial libraries. Adv Immunol 57:191-280.
Cai et al. (2020) Distinct conformational states of SARS-CoV-2 spike protein. Science 369:1586-1592.
Cantuti-Castelvetri et al. (2020) Neuropilin-1 facilitates SARS-CoV-2 cell entry and infectivity. Science 370:856-860.
Cao et al. (2020) De novo design of picomolar SARS-CoV-2 miniprotein inhibitors. Science 370:426-431.
Carter et al. (1992a) High level Escherichia coli expression and production of a bivalent humanized antibody fragment. Bio/Technology 10:163-167.
Carter et al. (1992b) Humanization of an anti-p185HER2 antibody for human cancer therapy. Proc Natl Acad Sci USA 89:4285.
Chatterjee et al. (2020) Targeted intracellular degradation of SARS-CoV-2 via computationally optimized peptide fusions. Commun Biol 3:715.
Cheng et al. (2020) Furin Inhibitors Block SARS-CoV-2 Spike Protein Cleavage to Suppress Virus Production and Cytopathic Effects. Cell Rep 33:108254.
Cho et al. (2021) Anti-SARS-CoV-2 receptor-binding domain antibody evolution after mRNA vaccination. Nature 600 (7889): 517-522.
Chothia & Lesk (1987) Canonical structures for the hypervariable regions of immunoglobulins. J Mol Biol 196:901-917.
Chou & Fasman (1974) Prediction of protein conformation. Biochemistry 13:222-245.
Chou & Fasman (1978) Empirical predictions of protein conformation. Ann Rev Biochem 47:251-276.
Chou & Fasman (1979) Prediction of beta-turns. Biophys J 26:367-384.
Cliffe et al. (2019) Filamentous Aggregates Are Fragmented by the Proteasome Holoenzyme. Cell Rep 26:2140-2149 e3.
Cobey et al. (2021) Concerns about SARS-CoV-2 evolution should not hold back efforts to expand vaccination. Nat Rev Immunol 21 (5): 330-305.
Cole et al. (1985) in Monoclonal Antibodies and Cancer Therapy, Alan R. Liss, Inc., New York, New York, United States of America, pp. 77-96.

### Consolidated References

Costabile (2010) Measuring the 50% Haemolytic Complement (CH50) Activity of Serum J Vis Exp 37: e1923.
Cote et al. (1983) Generation of human monoclonal antibodies reactive with cellular antigens. Proc Natl Acad Sci USA 80:2026-2030.
Crawford et al. (2020) Protocol and Reagents for Pseudotyping Lentiviral Particles with SARS-CoV-2 Spike Protein for Neutralization Assays. Viruses 12:513.
de Araujo et al. (2015) Isolation of Early and Late Endosomes by Density Gradient Centrifugation. Cold Spring Harb Protoc 2015:1013-1016.
de Kruif et al. (1995) Selection and application of human single chain Fv antibody fragments from a semi-synthetic phage antibody display library with designed CDR3 regions. J Mol Biol 248:97-105.
Deutscher et al. (ed.) (1990) Guide to Protein Purification, Harcourt Brace Jovanovich, San Diego, California, United States of America.
Devereux et al. (1984) A comprehensive set of sequence analysis programs for the VAX. Nucl Acids Res 12:387-395.
Dinnon 3^{rd} (2020) A mouse-adapted model of SARS-CoV-2 to test COVID-19 countermeasures. Nature 586 (7830): 560-566.
Doorduijn et al. (2017) Creating model systems to study molecular mechanisms of C5 convertase-induced Membrane Attack Complex assembly. Abstracts/Molecular Immunology. 89:160.
Durocher & Butler (2009) Expression systems for therapeutic glycoprotein production. Curr Opin Biotechnol 20:700-707.
Eyre et al. (2022) Effect of Covid-19 Vaccination on Transmission of Alpha and Delta Variants. New Engl J Med 386 (8): 744-756.
Fattizzo et al. (2020) Second-Generation C5 Inhibitors for Paroxysmal Nocturnal Hemoglobinuria. BioDrugs. 34 (2): 149-158.
Genaro (1985) Remington's Pharmaceutical Sciences, Mack Publishing Co., Easton, Pennsylvania, United States of America.
Ghosh et al. (2020) beta-Coronaviruses Use Lysosomes for Egress Instead of the Biosynthetic Secretory Pathway. Cell 183:1520-1535 e14.
Glasgow et al. (2020) Engineered ACE2 receptor traps potently neutralize SARS-CoV-2. Proc Natl Acad Sci USA 117:28046-28055.
Gorham et al. (2017) Functional characterization of alternative pathway C3 and C5 convertase inhibitors. Abstracts/Molecular Immunology 89:141.
Green & Sambrook (2012) Molecular Cloning: A Laboratory Manual, Fourth Edition, Cold Spring Harbor Laboratory Press, Cold Spring Harbor, New York, United States of America.
Gu et al. (1997) Construction and expression of mouse-human chimeric antibody SZ-51 specific for activated platelet P-selectin. Thromb Haemost 77 (4): 755-759.
Han et al. (2022) Receptor binding and complex structures of human ACE2 to spike RBD from omicron and delta SARS-CoV-2. Cell 185 (4): 630-640.
Harlow & Lane (1988) Antibodies, A Laboratory Manual, Cold Spring Harbor Laboratory Publications, Cold Spring Harbor, New York, United States of America.
Hoare et al. (2020) The importance of target binding kinetics for measuring target binding affinity in drug discovery: a case study from a CRF1 receptor antagonist program. Drug Discov Today 25:7-14.
Hoffmann et al. (2020a) A Multibasic Cleavage Site in the Spike Protein of SARS-CoV-2 Is Essential for Infection of Human Lung Cells. Mol Cell 78:779-784 e5.
Hoffmann et al. (2020b) SARS-CoV-2 Cell Entry Depends on ACE2 and TMPRSS2 and Is Blocked by a Clinically Proven Protease Inhibitor. Cell 181:271-280 e8.
Holter et al. (2020) Systemic complement activation is associated with respiratory failure in COVID-19 hospitalized patients. Proc Natl Acad Sci USA 117 (40): 25018-25025.
Hossain et al. (2022) Roles of the polybasic furin cleavage site of spike protein in SARS-CoV-2 replication, pathogenesis, and host immune responses and vaccination. J Med Virol 94 (5): 1815-1820.
Huse et al. (1989) Generation of a large combinatorial library of the immunoglobulin repertoire in phage lambda. Science 246:1275-1281.
Huston et al. (1988) Protein engineering of antibody binding sites: recovery of specific activity in an anti-digoxin single-chain Fv analogue produced in Escherichia coli. Proc Natl Acad Sci USA 85:5879-5883.
Iwata-Yoshikawa et al. (2022) A lethal mouse model for evaluating vaccine-associated enhanced respiratory disease during SARS-CoV-2 infection. Sci Adv 8 (1): eabh3827.
Izidoro et al. (2009) A study of human furin specificity using synthetic peptides derived from natural substrates, and effects of potassium ions. Arch Biochem Biophys 487:105-114.
Jackson et al. (2021) Mechanisms of SARS-CoV-2 entry into cells. Nature Reviews Molecular Cell Biology 23:3-20.
Jaimes et al. (2019) A Fluorogenic Peptide Cleavage Assay to Screen for Proteolytic Activity: Applications for coronavirus spike protein activation. J Vis Exp doi: 10.3791/58892.
Jodele et al. (2020) Complement blockade for TA-TMA: lessons learned from a large pediatric cohort treated with eculizumab. Blood 135 (13): 1049-1057.
Johnson et al. (2021) Loss of furin cleavage site attenuates SARS-CoV-2 pathogenesis. Nature 591:293-299.
Jones et al. (1986) Replacing the complementarity-determining regions in a human antibody with those from a mouse. Nature 321:522-525.
Kacprzak et al. (2004) Inhibition of furin by polyarginine-containing peptides: nanomolar inhibition by nona-D-arginine. J Biol Chem 279:36788-94.
Kalathiya et al. (2020) Highly Conserved Homotrimer Cavity Formed by the SARS-CoV-2 Spike Glycoprotein: A Novel Binding Site. J Clin Med 9:1473.
Karlin & Altschul (1990) Methods for assessing the statistical significance of molecular sequence features by using general scoring schemes. Proc Natl Acad Sci USA 87:2264-2268.
Karlin & Altschul (1993) Applications and statistics for multiple high-scoring segments in molecular sequences. Proc Natl Acad Sci USA 90:5873-5877.
Karmon & Ben Aroya (2019) Spatial Organization of Proteasome Aggregates in the Regulation of Proteasome Homeostasis. Front Mol Biosci 6:150.
Kleine-Weber et al. (2019) Mutations in the Spike Protein of Middle East Respiratory Syndrome Coronavirus Transmitted in Korea Increase Resistance to Antibody-Mediated Neutralization. J Virol 93: e01381-18.
Klimpel et al. (1992) Anthrax toxin protective antigen is activated by a cell surface protease with the sequence specificity and catalytic properties of furin. Proc Natl Acad Sci U SA 89:10277-10281.
Kohler & Milstein (1975) Continuous cultures of fused cells secreting antibody of predefined specificity. Nature 256:495-497.
Konner et al. (2010) Farletuzumab, a humanized monoclonal antibody against folate receptor alpha, in epithelial ovarian cancer: a phase I study. Clin Cancer Res 16:5288-5295.
Koo et al. (2006) Cell-surface processing of pro-ADAMTS9 by furin. J Biol Chem 281:12485-12494.
Kozbor & Roder (1983) The production of monoclonal antibodies from human lymphocytes. Immunology Today 4:72.
Kozlov (2021) Omicron overpowers key COVID antibody treatments in early tests. Nature: doi: 10.1038/d41586-021-03829-0.
Kyte & Doolittle (1982) A simple method for displaying the hydropathic character of a protein. J Mol Biol 157:105-132.
Lan et al. (2020) Structure of the SARS-CoV-2 spike receptor-binding domain bound to the ACE2 receptor. Nature 581:215-220.
Latuszek et al. (2020) Inhibition of complement pathway activation with Pozelimab, a fully human antibody to complement component C5. PLOS One 15 (5): e0231892.
Liu et al. (2020) Potent neutralizing antibodies against multiple epitopes on SARS-CoV-2 spike. Nature 584:450-456.
Lu et al. (2006) Severe acute respiratory syndrome-associated coronavirus 3a protein forms an ion channel and modulates virus release. Proc Natl Acad Sci USA 103:12540-12545.
Lum & Tushir-Singh (2021) Arming “old guards” with “new dual-targeting weapons”. Cancer Cell 39:604-606.
Ma et al. (2021) Increased complement activation is a distinctive feature of severe SARS-CoV-2 infection. Sci Immunol 6 (59): eabh2259.
Markov et al. (2022) Antigenic evolution will lead to new SARS-CoV-2 variants with unpredictable severity. Nat Rev Microbiol 20 (5): 251-252.
Marks et al. (1991) By-passing immunization: human antibodies from V-gene libraries displayed on phage. J Mol Biol 222:581-597.
Matheson et al. (2020) How does SARS-CoV-2 cause COVID-19?. Science 369 (6503): 510-511.
McCallum et al. (2020) Structure-guided covalent stabilization of coronavirus spike glycoprotein trimers in the closed conformation. Nat Struct Mol Biol 27:942-949.
Mohammed et al. (2022) The efficacy and effectiveness of the COVID-19 vaccines in reducing infection, severity, hospitalization, and mortality: a systematic review. Hum Vacc Immunother 2022; 18 (1): Article 2027160.
Mondal et al. (20221) A Feasible Alternative Strategy Targeting Furin Disrupts SARS-CoV-2 Infection Cycle. Microbiol Spectr 10 (1): e02364-21.
Mondal et al. (2021b) Unexpected PD-L1 immune evasion mechanism in TNBC, ovarian, and other solid tumors by DR5 agonist antibodies. EMBO Mol Med 13: e12716.
Morimoto & Inouye (1992) Single-step purification of F(ab′) 2 fragments of mouse monoclonal antibodies (immunoglobulins G1) by hydrophobic interaction high performance liquid chromatography using TSKgel Phenyl-5 PW. J Biochem Biophys Methods 24:107-117.
Morrison et al. (1984) Chimeric human antibody molecules: mouse antigen-binding domains with human constant region domains. Proc Natl Acad Sci USA. 81:6851-6855.
Neuberger et al. (1984) Recombinant antibodies possessing novel effector functions. Nature 312:604-608.
Oganesyan et al. (2014) Structural insights into neonatal Fc receptor-based recycling mechanisms. J Biol Chem 289:7812-24.
Omotuyi (2015) Ebola virus envelope glycoprotein derived peptide in human Furin-bound state: computational studies. J Biomol Struct Dyn 33:461-70.
Park et al. (2017) The Endoplasmic Reticulum Chaperone GRP78/BiP Modulates Prion Propagation in vitro and in vivo. Sci Rep 7:44723.
Park et al. (2018) Next-generation proteasome inhibitors for cancer therapy. Transl Res. 2018; 198:1-16.
Paul (1993) Fundamental Immunology, 3rd Ed., Raven Press, New York, New York, United States of America.
PCT International Patent Application Publication Nos. WO 1992/02190; WO 1993/16185; WO 2019/099374; WO 2020/010107; WO 2021/150770.
Presta (1992) Antibody engineering. Curr Op Struct Biol 2:593-596.
Presta et al. (1993) Humanization of an antibody directed against IgE. J Immunol 1993 151:2623.
Przanowski et al. (2020) Oncogenic TRIM37 Links Chemoresistance and Metastatic Fate in Triple-Negative Breast Cancer. Cancer Res 80 (21): 4791-4804.
Puhach et al. (2022) Infectious viral load in unvaccinated and vaccinated individuals infected with ancestral, Delta or Omicron SARS-CoV-2. Nat Med 28 (7): 1491-1500.
Ramlall et al. (2020) Immune complement and coagulation dysfunction in adverse outcomes of SARS-CoV-2 infection. Nature Medicine 26 (10): 1609-1615.
Razonable et al. (2021) Casirivimab-Imdevimab treatment is associated with reduced rates of hospitalization among high-risk patients with mild to moderate coronavirus disease-19. EClinicalMedicine. 40:101102.
Read et al. (2015) Imperfect Vaccination Can Enhance the Transmission of Highly Virulent Pathogens. PLOS Biol 13 (7): e1002198.
Richman (2021) COVID-19 vaccines: implementation, limitations and opportunities. Glob Health Med 3 (1): 1-5.
Ridgway et al. (1996) ‘Knobs-into-holes’ engineering of antibody CH3 domains for heavy chain heterodimerization. Protein Eng 9 (7): 617-621.
Riechmann et al. (1988) Reshaping human antibodies for therapy. Nature 332:323-327.
Ritchie et al. (2013) Implications of receptor-mediated endocytosis and intracellular trafficking dynamics in the development of antibody drug conjugates. MAbs 5:13-21.
Rittirsch et al. (2012) Role of complement in multiorgan failure. Clin Dev Immunol 2012:962927.
Robertson et al. (2018) Structure of the complement C5a receptor bound to the extra-helical antagonist NDT9513727. Nature 553 (7686): 111-114.
Rogers et al. (2020) Isolation of potent SARS-CoV-2 neutralizing antibodies and protection from disease in a small animal model. Science 369:956-963.
Rondeau et al. (2021) Corrigendum to “Rondeau et al; on behalf of the 311 Study Group. The long-acting C5 inhibitor, Ravulizumab, is effective and safe in adult patients with atypical hemolytic uremic syndrome naïve to complement inhibitor treatment.” Kidney Int. 2020; 97:1287-1296. Kidney Int 2021; 99 (5): 1244.
Sahu et al. (2022) Increased complement activation is a distinctive feature of severe SARS-CoV-2 infection. Molecular Immunology 141:189.
Santiesteban-Lores et al. (2021) A double edged-sword—The Complement System during SARS-CoV-2 infection. Life Sci 272:119245.
Saunders (2019) Conceptual Approaches to Modulating Antibody Effector Functions and Circulation Half-Life. Front Immunol 10:1296.
Savitt et al.) 2021) SARS-CoV-2 Exacerbates COVID-19 Pathology Through Activation of the Complement and Kinin Systems. Frontiers in Immunology 12:767347.
Scolding et al. (1989) Vesicular removal by oligodendrocytes of membrane attack complexes formed by activated complement. Nature 339 (6226): 620-622.
Sefik et al. (2021) A humanized mouse model of chronic COVID-19. Nat Biotechnol 40 (6): 906-920.
Shivange et al. (2018) A Single-Agent Dual-Specificity Targeting of FOLR1 and DR5 as an Effective Strategy for Ovarian Cancer. Cancer Cell 34:331-345 e11.
Shivange et al. (2020) Analyzing Tumor and Tissue Distribution of Target Antigen Specific Therapeutic Antibody. J Vis Exp doi: 10.3791/60727.
Shivange et al. (2021) A patch of positively charged residues regulates the efficacy of clinical DR5 antibodies in solid tumors. Cell Rep 37:109953.
Sims et al. (1993) A humanized CD18 antibody can block function without cell destruction. J Immunol 151:2296-2308.
Takeda et al. (1985) Construction of chimaeric processed immunoglobulin genes containing mouse variable and human constant region sequences. Nature 314:452-454.
Thai & Ogata (2005) Recombinant C345C and factor I modules of complement components C5 and C7 inhibit C7 incorporation into the complement membrane attack complex, Journal of Immunology 174 (10): 6227-6232.
Thomas (2002) Furin at the cutting edge: from protein traffic to embryogenesis and disease. Nat Rev Mol Cell Biol 3:753-766.
Tian et al. (2012) Computational prediction of furin cleavage sites by a hybrid method and understanding mechanism underlying diseases. Sci Rep 2:261.
Tsuneoka et al. (1993) Evidence for involvement of furin in cleavage and activation of diphtheria toxin. J Biol Chem 268:26461-26465.
Tushir-Singh (2017) Antibody-siRNA conjugates: drugging the undruggable for anti-leukemic therapy. Expert Opin Biol Ther 17 (3): 325-338.
Tuszynski et al. (1988) Thrombospondin promotes platelet aggregation. Blood 72:109-115.
U.S. Patent Application Publication Nos. 2002/0034765, 2003/0017534, 2003/0153043, 2003/0022244, 2004/0253645, 2006/0073137, 2018/0298087, 2018/0312588, 2018/0346564, and 2019/0151448.
U.S. Pat. Nos. 4,554,101; 4,816,567; 4,946,778; 4,975,369; 5,075,431; 5,081,235; 5,169,939; 5,202,238; 5,204,244; 5,225,539; 5,231,026; 5,292,867; 5,354,847; 5,436,157; 5,472,693; 5,482,856; 5,491,088; 5,500,362; 5,502,16; 5,530,101; 5,571,894; 5,585,089; 5,587,458; 5,641,870; 5,693,761; 5,693,762; 5,712,120; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,479,284; 6,548,640; 6,632,927; 6,639,055; 6,677,436; 6,750,325; 6,797,492; 7,060,808; 7,906,625; 8,398,980; 8,436,150; 8,796,439; and 10,253,111.
Verhoeyen et al. (1988) Reshaping human antibodies: grafting an antilysozyme activity. Science 239:1534-1536.
Villoutreix et al. (2021) In Silico Investigation of the New UK (B.1.1.7) and South African (501Y.V2) SARS-CoV-2 Variants with a Focus at the ACE2-Spike RBD Interface. Int J Mol Sci 22:1695.
Wang et al. (2021) Antibody Resistance of SARS-CoV-2 Variants B.1.351 and B.1.1.7. Nature 593:130-135.
Weiss et al. (2021) Am J Trop Med Hyg 105 (5): 53-54.
Whitt (2010) Generation of VSV pseudotypes using recombinant DeltaG-VSV for studies on virus entry, identification of entry inhibitors, and immune responses to vaccines. J Virol Methods 169:365-374.
Whittaker (2021) SARS-CoV-2 spike and its adaptable furin cleavage site. Lancet Microbe 2 (10): e488-e9.
Winter & Milstein (1991) Man-made antibodies. Nature 349:293-299.
Wrapp et al. (2020) Cryo-EM Structure of the 2019-nCOV Spike in the Prefusion Conformation. Science 367:1260-1263.
Wright et al. (1992) Genetically engineered antibodies: progress and prospects. Critical Rev in Immunol 12 (3,4): 125-168.
Xia et al. (2020) Inhibition of SARS-CoV-2 (previously 2019-nCOV) infection by a highly potent pan-coronavirus fusion inhibitor targeting its spike protein that harbors a high capacity to mediate membrane fusion. Cell Res 30:343-355.
Yu et al. (2020) Mechanistic Insights to the Binding of Antibody CR3022 Against RBD from SARS-CoV and HCOV-19/SARS-CoV-2: A Computational Study. Comb Chem High Throughput Screen 24:1069-1082.
Yuan et al. (2020) A highly conserved cryptic epitope in the receptor binding domains of SARS-CoV-2 and SARS-CoV. Science 368:630-633.
Zelek et al. (2020a) Characterizing the original anti-C5 function-blocking antibody, BB5.1, for species specificity, mode of action and interactions with C5. Immunology 161 (2): 103-113.
Zelek et al. (2020b) Monoclonal Antibodies Capable of Inhibiting Complement Downstream of C5 in Multiple Species. Front Immunol 11:612402.
Zhang et al. (2015) 3D Structural Fluctuation of IgG1 Antibody Revealed by Individual Particle Electron Tomography. Sci Rep 5:9803.
Zwarthoff et al. (2018) Functional Characterization of Alternative and Classical Pathway C3/C5 Convertase Activity and Inhibition Using Purified Models. Frontiers in Immunology 9:1691.

TABLE 3
Representative Antibody Sequences (Predicted CDRs in Bold)

22 VL (SEQ ID NO: 1)
DIQLTQSPDSLAVSLGERATINCKSSQSVLYSSINKNYLAWYQQKPGQPPKLLIY
WASTRESGVPDRFSGSGSGTDFTLTISSLQAEDVAVYYCQQYYSTPYTFGQGTKV
EIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
22VH (SEQ ID NO: 5)
QLVQSGTEVKKPGESLKISCKGSGYGFITYWIGWVRQMPGKGLEWMGIIYPGDSE
TRYSPSFQGQVTISADKSINTAYLQWSSLKASDTAIYYCAGGSGISTPMDVWGQG
TTVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSC
DKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKS
LSLSLGK
22VH-FUG1 (SEQ ID NO: 9)
QLVQSGTEVKKPGESLKISCKGSGYGFITYWIGWVRQMPGKGLEWMGIIYPGDSE
TRYSPSFQGQVTISADKSINTAYLQWSSLKASDTAIYYCAGGSGISTPMDVWGQG
TTVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSC
DKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKS
LSLSLGKEGGGSGRERKARGGCPGS
22VH-cFUG1 (SEQ ID NO: 10)
QLVQSGTEVKKPGESLKISCKGSGYGFITYWIGWVRQMPGKGLEWMGIIYPGDSE
TRYSPSFQGQVTISADKSINTAYLQWSSLKASDTAIYYCAGGSGISTPMDVWGQG
TTVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSC
DKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKS
LSLSLGKEGGSSGRSRRARSVASQS
6.29VL (SEQ ID NO: 11)
DIQLTQSPSSLSASVGDRVTITCRASQTASSYLNWYQQKPGKAPNLLIYAASSLQ
SGVPSRFSGSGSVTDFTLTISSLQPEDFATYYCQQSYSTPPTFGQGTKVDIKRTV
AAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
6.29VH (SEQ ID NO: 15)
QVQLVQSGSELKKPGASVKVSCKASGYTFATYALNWVRQAPGQGLEWMGWVNTNT
GSPTYAQGFTGRFVFSFDTSVSTAYLQIRTLKAEDTAVYYCAVYYYDSGSPGWFD
PWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNH
YTQKSLSLSLGK
6.29VH-FuG1 (SEQ ID NO: 19)
QVQLVQSGSELKKPGASVKVSCKASGYTFATYALNWVRQAPGQGLEWMGWVNTNT
GSPTYAQGFTGRFVFSFDTSVSTAYLQIRTLKAEDTAVYYCAVYYYDSGSPGWFD
PWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNH
YTQKSLSLSLGKEGGGSGRERKARGGCPGS
6.30VL (SEQ ID NO: 20)
DIQMTQSPSSLSASVGDRVTITCRASQNISSYLNWYQQEAGKAPKLLIYAASSLQ
SGVPSRFSGSGSGTDFTLTISSLQPEDFATYYCQQSYSTPRTFGQGTKVDIKRTV
AAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
6.30VH (SEQ ID NO: 23)
QVQLVQSGAEVKKPGSSVKVSCKASGGTFSIYAITWVRQAPGQGLEWMGGIIPII
GTANYAQKFQGRVTITADKSTSTAYMELSSLRSEDTAVYYCARDFRYCSSTRCYF
WFDPWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEAL
HNHYTQKSLSLSLGK
6.30VH-FuG1 (SEQ ID NO: 27)
QVQLVQSGAEVKKPGSSVKVSCKASGGTFSIYAITWVRQAPGQGLEWMGGIIPII
GTANYAQKFQGRVTITADKSTSTAYMELSSLRSEDTAVYYCARDFRYCSSTRCYF
WFDPWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEAL
HNHYTQKSLSLSLGKEGGGSGRERKARGGCPGS
12.25 VL (SEQ ID NO: 28)
QSALTQPPSASGTPGQRVTISCSGSSSNIGSNTVNWYQQLPGTAPKVLVYSNDQR
PSGVPDRFSGSKSGTSASLAISGLQSEDEADYYCAAWDDSLNGPVFGGGTKLTVL
GQPKAAPSVTLFPPSSEELQANKATLVCLISDFYPGAVTVAWKADSSPVKAGVET
TTPSKQSNNKYAASSYLSLTPEQWKSHRSYSCQVTHEGSTVEKTVAPTECS
12.25 VH (SEQ ID NO: 32)
QVQLVESGGGLVQPGGSLRLSCAASGFTFSSYAMSWVRQAPGKGLEWVSAISGSG
DSTYYADSVKGRFTISRDNSKNTLYLQMNSLRAEDTAVYYCAKDRYYEFWSGYSN
WFDPWGQGTLVTISSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEAL
HNHYTQKSLSLSLGK
12.25 VH-FuG1 (SEQ ID NO: 36)
QVQLVESGGGLVQPGGSLRLSCAASGFTFSSYAMSWVRQAPGKGLEWVSAISGSG
DSTYYADSVKGRFTISRDNSKNTLYLQMNSLRAEDTAVYYCAKDRYYEFWSGYSN
WFDPWGQGTLVTISSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEAL
HNHYTQKSLSLSLGKEGGGSGRERKARGGCPGS
FuG1 Fc-Extended Peptide (SEQ ID NO: 44)
EGGGSGRERKARGGCPGS
cFuG1 Fc-Extended Peptide (SEQ ID NO: 45)
EGGSSGRSRRARSVASQS
KMTR2 c-kappa (VL) (SEQ ID NO: 46)
EIVLTQSPATLSLSPGERATLSCRASQSVSSYLAWYQQKPGQAPRLLIYDASNRA
TGIPARFSGSGSGTDFTLTISSLEPEDFAVYYCQQRSNWPLTFGGGTKVEIKRTV
AAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
KMTR2 IgG1 (VH) (SEQ ID NO: 47)
QVQLVQSGAEMKKPGASVKVSCKTSGYTFTNYKINWVRQAPGQGLEWMGWMNPDT
DSTGYPQKFQGRVTMTRNTSISTAYMELSSLRSEDTAVYYCARSYGSGSYYRDYY
YGMDVWGQGTTVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKV
DKKVEPKSCDKTHTCPPCPAPEAAGGPSVFLFPPKPKDTLMISRTPEVTCVVVDV
SHEDPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCSVMHEA
LHNHYTQKSLSLSPGK
Avelumab c-kappa (VL) (SEQ ID NO: 48)
MGWSCIILFLVATATGVHSQSALTQPASVSGSPGQSITISCTGTSSDVGGYNYVS
WYQQHPGKAPKLMIYDVSNRPSGVSNRFSGSKSGNTASLTISGLQAEDEADYYCS
SYTSSSTRVFGTGTKVTVLRTVAAPSVFIFPPSDEQLKSGTASVVCLLNNFYPRE
AKVQWKVDNALQSGNSQESVTEQDSKDSTYSLSSTLTLSKADYEKHKVYACEVTH
QGLSSPVTKSFNRGEC
Avelumab IgG4 (VH) (SEQ ID NO: 49)
MGWSCIILFLVATATGVHSEVQLLESGGGLVQPGGSLRLSCAASGFTFSSYIMMW
VRQAPGKGLEWVSSIYPSGGITFYADTVKGRFTISRDNSKNTLYLQMNSLRAEDT
AVYYCARIKLGTVTTVDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTY
ICNVNHKPSNTKVDKKVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLT
CLVKGFYPSDIAVEWESNGQPENNYKTTPPVLDSDGSFFLYSKLTVDKSRWQEGN
VFSCSVMHEALHNHYTQKSLSLSLG
Farletuzumab c-kappa (VL) (SEQ ID NO: 50)
DIQLTQSPSSLSASVGDRVTITCSVSSSISSNNLHWYQQKPGKAPKPWIYGTSNL
ASGVPSRFSGSGSGTDYTFTISSLQPEDIATYYCQQWSSYPYMYTFGQGTKVEIK
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
Farletuzumab IgG1 (VH) (SEQ ID NO: 51)
EVQLVESGGGVVQPGRSLRLSCSASGFTFSGYGLSWVRQAPGKGLEWVAMISSGG
SYTYYADSVKGRFAISRDNAKNTLFLQMDSLRPEDTGVYFCARHGDDPAWFAYWG
QGTPVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPK
SCDKTHTCPPCPAPEAAGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQ
KSLSLSLPGK

It will be understood that various details of the presently disclosed subject matter can be changed without departing from the scope of the presently disclosed subject matter. Furthermore, the foregoing description is for the purpose of illustration only, and not for the purpose of limitation.

1. A method for treating a viral infection in a subject in need thereof, the method comprising administering to the subject a composition comprising an effective amount of an agent that selectively interferes with host protease function to inhibit fusion-ready viral fragment generation, optionally S2 in case of SARS-CoV2 or GP160 or GP120 in case of HIV, and/or to destabilize a full-length viral fusion protein, optionally SARS-CoV-2 spike.

2. The method of claim 1, wherein the composition comprises an effective amount of an agent that selectively interferes with host furin protease function to inhibit fusion-ready S2 fragment generation and/or to destabilize full-length spike (S0) protein.

3. The method of claim 1, wherein the viral infection is a HIV infection or a coronavirus infection, such as but not limited to Delta variant, Omicron variant, or Deltacron variant.

4. The method of claim 1, wherein the viral infection is a SAR-CoV-2 infection, such as but not limited to Delta variant, Omicron variant, or Deltacron variant.

5. The method of claim 1, wherein the agent comprises an Fc-conjugated furin competitive peptide and an antibody, optionally wherein the agent comprises a sequence as set forth in Table 3, or a biologically active fragment and/or homolog thereof, further optionally wherein the Fc-conjugated peptide comprises a sequence as set forth in Table 3 or a biologically active fragment and/or homolog thereof.

6. The method of claim 1, wherein the agent comprises an Fc-conjugated furin competitive peptide and an antibody, and the Fc-conjugated furin competitive peptide is uncleavable by furin.

7. The method of claim 1, wherein the agent comprises an Fc-conjugated furin competitive peptide and an antibody, wherein the Fc-conjugated furin competitive peptide is stable, flexible, and can be conjugated to any antibody targeting a virus.

8. The method of claim 1, wherein the agent comprises an Fc-conjugated furin competitive peptide and an antibody, wherein the antibody Fc-conjugated furin competitive peptide is against furin recognition sequence of gp160 or gp120 of HIV or spike of SARS-CoV-2.

9. The method of claim 1, wherein the antibody comprises a light chain variable region and a heavy chain variable region, and further wherein the light chain variable region comprises one of SEQ ID NOs: 1, 11, 20, and 28 or a sequence at least 95% identical thereto, and the heavy chain variable region comprises one of SEQ ID NOs: 5, 15, 23, and 32 or a sequence at least 95% identical thereto.

10. The method of claim 1, wherein:
the antibody comprises a light chain variable region comprising SEQ ID NO: 1 and a heavy chain variable region comprising SEQ ID NO: 5; or
the antibody comprises a light chain variable region comprising SEQ ID NO: 11 and a heavy chain variable region comprising SEQ ID NO: 15; or
the antibody comprises a light chain variable region comprising SEQ ID NO: 20 and a heavy chain variable region comprising SEQ ID NO: 23; or
the antibody comprises a light chain variable region comprising SEQ ID NO: 28 and a heavy chain variable region comprising SEQ ID NO: 32.

11. The method of claim 1, wherein the heavy chain further comprises a C-terminal peptide selected from the group consisting of SEQ ID NOs: 44 and 45.

12. The method of claim 1, wherein the antibody comprises a complement component antibody or a biologically active fragment and/or homolog thereof, optionally wherein the complement component antibody comprises an anti-C5 antibody or a biologically active fragment and/or homolog thereof, optionally wherein the complement component antibody comprises ravulizumab or eculizumab, or a biologically active fragment and/or homolog thereof, further optionally wherein the complement component antibody comprises SEQ ID NO: 65 and/or SEQ ID NO: 66, or a biologically active fragment and/or homolog thereof, further optionally wherein the biologically active fragment and/or homolog is at least 95% identical to SEQ ID NO: 65 or 66.

13. A composition comprising an effective amount of an agent that selectively interferes with host protease function to inhibit fusion-ready viral fragment generation, optionally S2 in case of SARS-CoV2 or GP160 or GP120 in case of HIV, and/or to destabilize a full-length viral fusion protein, optionally SARS-CoV-2 spike.

14. The composition of claim 13, wherein the composition comprises an effective amount of an agent that selectively interferes with host furin protease function to inhibit fusion-ready S2 fragment generation and/or to destabilize full-length spike (S0) protein.

15. The composition of claim 13, wherein the agent comprises an Fc-conjugated furin competitive peptide and an antibody, optionally wherein the agent comprises a sequence as set forth in Table 3, or a biologically active fragment and/or homolog thereof, further optionally wherein the Fc-conjugated peptide comprises a sequence as set forth in Table 3, or a biologically active fragment and/or homolog thereof.

16. The composition of claim 13, wherein the agent comprises an Fc-conjugated furin competitive peptide and an antibody, and the Fc-conjugated furin competitive peptide is uncleavable by furin.

17. The composition of claim 13, wherein the agent comprises an Fc-conjugated furin competitive peptide and an antibody, and the Fc-conjugated furin competitive peptide is stable, flexible and can be conjugated to any antibody targeting a virus.

18. The composition of claim 13, wherein the agent comprises an Fc-conjugated furin competitive peptide and an antibody, and the antibody Fc-conjugated furin competitive peptide is against furin recognition sequence of gp160 or gp120 of HIV or spike of SARS-CoV-2.

19. The composition of claim 13, wherein the antibody comprises a light chain variable region and a heavy chain variable region, and further wherein the light chain variable region comprises one of SEQ ID NOs: 1, 11, 20, and 28 or a sequence at least 95% identical thereto, and the heavy chain variable region comprises one of SEQ ID NOs: 5, 15, 23, and 32 or a sequence at least 95% identical thereto.

20. The composition of claim 13, wherein:
the antibody comprises a light chain variable region comprising SEQ ID NO: 1 and a heavy chain variable region comprising SEQ ID NO: 5; or
the antibody comprises a light chain variable region comprising SEQ ID NO: 11 and a heavy chain variable region comprising SEQ ID NO: 15; or
the antibody comprises a light chain variable region comprising SEQ ID NO: 20 and a heavy chain variable region comprising SEQ ID NO: 23; or
the antibody comprises a light chain variable region comprising SEQ ID NO: 28 and a heavy chain variable region comprising SEQ ID NO: 32.

21. The composition of claim 13, wherein the heavy chain further comprises a C-terminal peptide selected from the group consisting of SEQ ID NOs: 44 and 45.

22. The composition of claim 13, further comprising a pharmaceutically acceptable carrier, optionally a pharmaceutically acceptable carrier for use in a human.

23. The composition of claim 13, for use in treating a viral infection and/or for use in treating COVID19, optionally long COVID19.

24. The composition of claim 23, wherein the viral infection is a HIV infection or a coronavirus infection, such as but not limited to Delta variant, Omicron variant, or Deltacron variant.

25. The composition of claim 23, wherein the viral infection is a SAR-CoV-2 infection, such as but not limited to Delta variant, Omicron variant, or Deltacron variant.

26. The composition of claim 13, wherein the antibody comprises a complement component antibody or a biologically active fragment and/or homolog thereof, optionally wherein the complement component antibody comprises an anti-C5 antibody or a biologically active fragment and/or homolog thereof, optionally wherein the complement component antibody comprises ravulizumab or eculizumab, or a biologically active fragment and/or homolog thereof, further optionally wherein the complement component antibody comprises SEQ ID NO: 65 and/or SEQ ID NO: 66, or a biologically active fragment and/or homolog thereof, further optionally wherein the biologically active fragment and/or homolog is at least 95% identical to SEQ ID NO: 65 or 66.

27. A method for treating COVID19 in a subject in need thereof, the method comprising administering to the subject a composition comprising an effective amount of an agent that selectively interferes with host protease function to inhibit fusion-ready viral fragment generation, optionally S2 in case of SARS-CoV2, and/or to destabilize a full-length viral fusion protein, optionally SARS-CoV-2 spike.

28. The method of claim 27, wherein the composition comprises an effective amount of an agent that selectively interferes with host furin protease function to inhibit fusion-ready S2 fragment generation and/or to destabilize full-length spike (S0) protein.

29. The method of claim 27, wherein the COVID19 is caused by a SARS-CoV-2 variant, such as but not limited to Delta variant, Omicron variant, or Deltacron variant.

30. The method of claim 27, wherein the agent comprises an Fc-conjugated furin competitive peptide and an antibody, optionally wherein the agent comprises a sequence as set forth in Table 3, or a biologically active fragment and/or homolog thereof, further optionally wherein the Fc-conjugated peptide comprises a sequence as set forth in Table 3 or a biologically active fragment and/or homolog thereof.

31. The method of claim 27, wherein the agent comprises an Fc-conjugated furin competitive peptide and an antibody, and the Fc-conjugated furin competitive peptide is uncleavable by furin.

32. The method of claim 27, wherein the agent comprises an Fc-conjugated furin competitive peptide and an antibody, wherein the Fc-conjugated furin competitive peptide is stable, flexible, and can be conjugated to any antibody targeting a virus.

33. The method of claim 27, wherein the agent comprises an Fc-conjugated furin competitive peptide and an antibody, wherein the antibody Fc-conjugated furin competitive peptide is against furin recognition sequence of spike of SARS-CoV-2.

34. The method of claim 27, wherein the antibody comprises a light chain variable region and a heavy chain variable region, and further wherein the light chain variable region comprises one of SEQ ID NOs: 1, 11, 20, and 28 or a sequence at least 95% identical thereto, and the heavy chain variable region comprises one of SEQ ID NOs: 5, 15, 23, and 32 or a sequence at least 95% identical thereto.

35. The method of claim 27, wherein:
the antibody comprises a light chain variable region comprising SEQ ID NO: 1 and a heavy chain variable region comprising SEQ ID NO: 5; or
the antibody comprises a light chain variable region comprising SEQ ID NO: 11 and a heavy chain variable region comprising SEQ ID NO: 15; or
the antibody comprises a light chain variable region comprising SEQ ID NO: 20 and a heavy chain variable region comprising SEQ ID NO: 23; or
the antibody comprises a light chain variable region comprising SEQ ID NO: 28 and a heavy chain variable region comprising SEQ ID NO: 32.

36. The method of claim 27, wherein the heavy chain further comprises a C-terminal peptide selected from the group consisting of SEQ ID NOs: 45 and 46.

37. The method of claim 27, wherein the agent comprises an antibody which comprises a complement component antibody or a biologically active fragment and/or homolog thereof, optionally wherein the complement component antibody comprises an anti C5 antibody or a biologically active fragment and/or homolog thereof, optionally wherein the complement component antibody comprises ravulizumab and eculizumab, or a biologically active fragment and/or homolog thereof, further optionally wherein the complement component antibody comprises SEQ ID NO: 65 and/or SEQ ID NO: 66, or a biologically active fragment and/or homolog thereof, such as a sequence at least 95% identical thereto.

38. The method of claim 27, any of claims 27-37, wherein the COVID19 is Long COVID19.
